(19) United States
(12) Patent Application Publication
(10) Pub. No.: US 2014/0163034 A1
(43) Pub. Date: Jun. 12, 2014

(54) INHIBITORS OF ANANDAMIDE TRANSPORT AND THEIR THERAPEUTIC USES

(71) Applicants: IIT - Istituto Italiano di Tecnologia, Genova (GE) (IT); The Regents of the University of California, Oakland, CA (US)

(72) Inventors: Daniele Piomelli, Irvine, CA (US); Jin Fu, Irvine, CA (US); Giovanni Bottegoni, Fano (IT); Andrea Cavalli, San Lazzaro di Savena (IT); Tiziano Bandiera, Gambolo (PV) (IT)

(73) Assignees: IIT-Istituto Italiano di Tecnologia, Genova (GE) (IT); The Regents of the University of California, Oakland, CA (US)

(21) Appl. No.: 14/089,387

(22) Filed: Nov. 25, 2013

Related U.S. Application Data
(63) Continuation of application No. PCT/US2012/040531, filed on Jun. 1, 2012.
(60) Provisional application No. 61/492,293, filed on Jun. 1, 2011.

(51) Int. Cl. C07D 237/34; C07K 14/435; G01N 33/50; C12N 15/11

(52) U.S. Cl. CPC C07D 237/34; C12N 15/11; C07K 14/435; G01N 33/5008
USPC 514/248; 536/23.5; 530/350; 435/7.1; 435/252.33; 435/254.2; 435/348; 435/349; 435/325

(57) ABSTRACT

Nucleic acid and polypeptide sequences corresponding to FLAT, a partly cytosolic variant of the intracellular anandamide-degrading enzyme, fatty acid amide hydrolase-1 (FAAH-1), are provided. FLAT lacks amidase activity but binds the endocannibinoid anandamide and facilitates its transport into cells. A chemical scaffold for the inhibition of anandamide transport is identified. Compositions of the invention prevent anandamide internalization in vitro, interrupt anandamide deactivation in vivo, and cause profound CB₁ cannabinoid receptor-mediated analgesia in a mouse model of inflammatory pain. Accordingly, the invention also provides methods, and pharmaceutical compositions for treating conditions in which the modulation of anandamide transport would be of benefit.

### CROSS-REFERENCES TO RELATED APPLICATIONS

This application is a continuation of International Patent Application No. PCT/US2012/040531, filed Jun. 1, 2012, which claims priority to U.S. Provisional Application Ser. No. 61/492,293 filed Jun. 1, 2011, the disclosure of each of which is incorporated herein by reference in its entirety and for all purposes.

### STATEMENT AS TO RIGHTS TO INVENTIONS MADE UNDER FEDERALLY SPONSORED RESEARCH AND DEVELOPMENT

This invention was made with government support under grant nos. RO1 DA012413, RO1 DA012447, and RL1 AA017538 awarded by the National Institutes of Health. The Government has certain rights in the invention.

### REFERENCE TO A SEQUENCE LISTING, A TABLE OR A COMPUTER PROGRAM LISTING APPENDIX SUBMITTED AS AN ASCII TEXT FILE

The Sequence Listing written in file 79828-842466_ST25.TXT, created on Nov. 24, 2013, 13,002 bytes, machine format IBM-PC, MS Windows operating system, is hereby incorporated by reference.

### BACKGROUND OF THE INVENTION

Pain perception can be effectively controlled by neurotransmitters that operate within the central nervous system (CNS). This modulation has been well characterized in the dorsal horn of the spinal cord, where impulses carried by nociceptive (pain-sensing) fibers are processed before they are transmitted to the brain. In addition to these central mechanisms, intrinsic control of pain transmission can occur at terminals of afferent nerve fibers outside the CNS. One prominent example of peripheral regulation is provided by the endogenous opioids, which are released from activated immune cells during inflammation and inhibit pain initiation by interacting with opioid receptors localized on sensory nerve endings (Stein, C. et al., Nat Med, 9 (8), 1003-1008 (2003); Stein, C. et al., Handb Exp Pharmacol, (194), 495-518 (2009).)

It has been proposed that endocannabinoid mediators might serve an analogous function to that of the opioids, because pharmacological activation of peripheral CB1 and CB2 cannabinoid receptors inhibits pain-related behaviors (Calignano, A. et al., Nature, 394 (6690), 277-281 (1998); Jaggar, S. I. et al., Neurosci Lett, 253 (2), 123-126 (1998); Nackley, A. G. et al., Neuroscience, 117 (3), 659-670 (2003); Dziadulewicz, E. K. et al., J Med Chem, 50 (16), 3851-3856 (2007); Anand, P. et al., Brain Res Rev, 60 (1), 255-266 (2009)) while genetic disruption of CB1 receptor expression in primary nociceptive neurons exacerbates such behaviors (Agarwal, N. et al., Nat Neurosci, 10 (7), 870-879 (2007)). Moreover, there is evidence that clinical conditions associated with neuropathic pain or inflammation, such as complex regional pain syndrome and arthritis, may be accompanied by peripheral elevations in the levels of the endocannabinoid anandamide (Kaufmann, I. et al., Eur Surg Res, 43 (4), 325-329 (2009); Richardson, D. et al., Arthritis Res Ther, 10 (2), R43 (2008)). Another major endocannabinoid ligand, 2-arachidonylglycerol (2-AG), has also been implicated in nociceptive signaling outside the CNS (Agarwal, N. et al., Nat Neurosci, 10 (7), 870-879 (2007); Mitrirattanakul, S. et al., Pain, 126 (1-3), 102-114 (2006)).

Anandamide, the naturally occurring amide of arachidonic acid with ethanolamine, meets all key criteria of an endogenous cannabinoid substance (Devane, W. A. et al. Science, 258, 1946-1949 (1992)): it is released upon demand by stimulated neurons (Di Marzo, V. et al., Nature, 372, 686-691 (1994); Giuffrida, A. et al., Nat. Neurosci., 2, 358-363 (1999)); it activates cannabinoid receptors with high affinity (Devane, W. A. et al. Science, 258, 1946-1949 (1992)) and it is rapidly eliminated through a two-step process consisting of carrier-mediated transport followed by intracellular hydrolysis (Di Marzo, V. et al., Nature, 372, 686-691 (1994); Beltramo, M. et al., FEBS Lett., 403, 263-267 (1997)). Anandamide hydrolysis is catalyzed by the enzyme fatty acid amide hydrolase (FAAH), a membrane-bound serine hydrolase (Cravatt, B. F. et al., Nature, 384, 83-87 (1996); Patricelli, M. P. et al., Biochemistry, 38, 9804-9812 (1999)) (WO 98/20119) (U.S. Pat. No. 6,271,015) that also cleaves other bioactive fatty ethanolamides, such as oleoylethanolamide (cis-9-octadecenamide)) (Rodriguez de Fonseca, F. et al. Nature, 414, 209-212 (2001)) and palmitoylethanolamide (Calignano, A. et al., Nature, 394, 277-281 (1998)).

Anandamide regulates ion-channel activity and neurotransmitter release by engaging CB₁-type cannabinoid receptors on axon terminals (Devane, W. A. et al., Science, 258, 1946 (1992)). There is evidence that the intensity and duration of anandamide signaling are controlled by a two-step elimination process in which the substance is first internalized by neurons and astrocytes (Beltramo, M. et al., Science, 277, 1094 (1997); Hillard, C. J. et al., J Neurochem, 69, 631 (1997); Hillard, C. J. et al., J Mol Neurosci, 33, 18 (2007)) and then hydrolyzed to arachidonic acid and ethanolamine by the intracellular membrane-bound amidases, fatty acid amide hydrolase-1 (FAAH-1) and FAAH-2 (Cravatt, B. F. et al., Proc Natl Acad Sci USA, 98, 9371 (2001); McKinney, M. K. et al., Annu Rev Biochem, 74, 411 (2005); Wei, B. Q. et al., J Biol Chem, 281, 36569 (2006)). Removal of anandamide from the extracellular space exhibits several identifying features of a carrier-mediated facilitated diffusion process (Hillard, C. J. et al., J Neurochem, 69, 631 (1997); Hillard, C. J. et al., Br J Pharmacol, 140, 802 (2003); Di Marzo, V. et al., Nat Rev Drug Discov, 7, 438 (2008)): (i) it is saturable and displays low micromolar affinity for anandamide (apparent Michaelis constant, K_{M}, 1.2 μM in rat cortical neurons) (Beltramo, M. et al., Science, 277, 1094 (1997)); (ii) it preferentially recognizes anandamide over structurally similar molecules, including the FAAH substrates oleoylethanolamide (OEA) and palmitoylethanolamide (PEA) (Beltramo, M. et al., Science, 277, 1094 (1997); Piomelli, D. et al., Proc Natl Acad Sci USA, 96, 5802 (1999)); (iii) it is inhibited in a competitive and stereoselective manner by substrate mimics (Di Marzo, V. et al., Nat Rev Drug Discov, 7, 438 (2008)); and (iv) it does not require cellular energy (Beltramo, M. et al., Science, 277, 1094 (1997); Hillard, C. J. et al., J Neurochem, 69, 631 (1997)). Anandamide transport inhibitors—which include the compounds AM404, VDM-11 and OMDM-1 (Di Marzo, V. et al., Nat Rev Drug Discov, 7, 438 (2008))—increase the levels of this endocannabinoid substance in vivo and produce a spectrum of CB₁ receptor-mediated responses that only partly overlap with those elicited by FAAH blockers (Di Marzo, V. et al., Nat Rev Drug Discov, 7, 438 (2008)), presumably owing to the different functional properties of the two deactivation mechanisms. These data indicate that carrier-mediated transport plays an important role in terminating the biological actions of anandamide and might represent a potential drug target (Di Marzo, V. et al., Nat Rev Drug Discov, 7, 438 (2008)).

Much attention has been directed toward the role of anandamide in pain. Methods of treating pain by administering anandamide and palmitoylethanolamide are disclosed in U.S. Patent Application Publication No.: 20020173550. Methods of treating pain by administering inhibitors of FAAH are disclosed in U.S. Patent Application Publication Nos. 20040127518 and 20030134894. Certain methods of treating pain including those disclosed in U.S. Patent Application Publication No. 20030149082, by administering inhibitors of anandamide transport. The transport process resulting in removal of anandamide from the extracellular space exhibits several identifying features of carrier-mediated facilitated diffusion, but the molecular entity involved in anandamide translocation is still unknown and the mechanistic bases of this process remain unclear. Although several anandamide transport inhibitors have been disclosed in the past (such as AM404, and VDM-11), these compounds were identified without molecular characterization of the anandamide transporter and their chemical scaffolds are very similar to anandamide. These first generation anandamide transport inhibitors are highly hydrophobic, very flexible, and possess limited druglikeness.

The present invention addresses these and other needs by identifying an effector of the membrane transport of anandamide, FLAT (FAAH1-Like Anandamide Transporter). The invention provides gene constructs for expression of FLAT in cultured neurons and other cells. The invention further provides isolated recombinant FLAT and means of identifying inhibitors of anandamide transport in vivo. Moreover, the invention provides a new chemical scaffold for the inhibition of anandamide transport and methods of its use in the treatment of a variety of conditions, including pain and/or inflammation.

### BRIEF SUMMARY OF THE INVENTION

In a first aspect, the invention provides pharmaceutical compositions comprising a therapeutically effective amount of the compound having the formula:

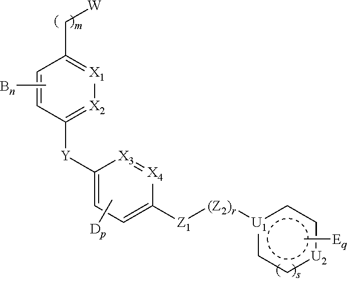

wherein:
W is aryl, heteroaryl, heterocycloalkyl, or alkyl, wherein the aryl, heteroaryl, or heterocycloalkyl can be substituted by 1 to 3 substituents selected from lower alkyl, alkenyl, OH, alkoxy, cyano, halogen, NR¹R², NR¹COR², CONR¹R², wherein R¹ and R² are independently selected from H or lower alkyl; m is an integer from 0 to 1; X₁, X₂, X₃, and X₄ are independently selected from carbon and nitrogen; n is an integer from 0 to 2; each B member is independently selected from the group consisting of alkoxy, alkyl, alkenyl, halogen and heteroalkyl, and, optionally, when n is 2 and two B members are on adjacent carbon atoms, the two adjacent B members may be taken together along with the atoms to which they are attached to form a saturated or unsaturated ring comprising 5 to 6 ring atoms; Y is independently selected from O, NR³, and C═O, wherein R³ is hydrogen or lower alkyl; p is an integer from 0 to 4; each D member is independently selected from the group consisting of alkoxy, alkyl, alkenyl, halogen, and heteroalkyl, and optionally when two D members are on adjacent carbon atoms, the adjacent D members may be taken together along with the atoms to which they are attached to form a saturated or unsaturated ring comprising 5 to 6 ring atoms; Z₁ and Z₂ are independently selected from C═O and NR⁴, wherein R⁴ is hydrogen or lower alkyl; r is an integer from 0 to 1; s is an integer from 0 to 1; U₁ is independently selected from C or N; U₂ is independently selected from C, O, and N(R⁵)ₜ, wherein the ring containing U₁ and U₂ can be aromatic, or partially or fully saturated, R⁵ is hydrogen or lower alkyl, and t is 0 or 1, with the proviso that when the ring containing U₁ and U₂ is aromatic, U₁ is C and t is 0; q is an integer from 0 to 4; each E member is independently selected from the group consisting of alkoxy, alkyl, alkenyl, halogen, and heteroalkyl, OH, cyano, NR⁶R⁷, wherein R⁶ and R⁷ are hydrogen or lower alkyl, and optionally when two E members are on adjacent carbon atoms the adjacent E members may be taken together along with the atoms to which they are attached to form a saturated or unsaturated ring comprising 5 to 6 ring atoms. The compositions can be formulated for any route of administration including the oral and parenteral routes. In addition, the compositions may be in a unit dose format. In addition, the inventions contains kits comprising multiple discrete dosages of the compounds in a single container. Preferably, the dosages are in unit dose format. In preferred embodiments, the compounds are inhibitors of anandamide transport thereby effectively increasing anandamide activity and/or levels in a subject administered a compound for use according to the invention.

In a second aspect, the invention provides a method of modulating anandamide activity in a subject by administering a composition or compound according to the invention. In some embodiments, the invention provides methods of treating conditions susceptible to therapy with anandamide transport inhibitors or with agents that increase cannabinoid activity. In some embodiments, the invention provides methods of treating pain, inflammation, anxiety, depression, sleep disorders, appetency disorders, attention deficit disorder, obsession or compulsion, schizophrenia, personality disorders, multiple sclerosis, nausea, or excessive body weight by administering a therapeutically effective amount of a compound according to the invention for the indicated use.

In a third aspect, the invention provides a cDNA encoding a partly cytosolic variant of the intracellular anandamide-degrading enzyme FAAH-1, termed FLAT, which lacks amidase activity but binds anandamide with low micromolar affinity and facilitates its transport into cells. The invention further provides a recombinant cell wherein the cell contains a heterologous nucleic acid encoding FLAT and the cell expresses FLAT. The invention provides gene constructs for expression of FLAT in cultured neurons and other cells. These cells may be used in assays to test compounds for FLAT activity. In certain embodiments, the invention provides an isolated FLAT protein.

In a fourth aspect the invention provides methods of screening compounds for FLAT inhibitory activity by contacting a recombinant FLAT protein in vivo or in vitro.

### BRIEF DESCRIPTION OF THE DRAWINGS

FIG. 1. Structural properties of FLAT. (A) Predicted amino acid sequences of FLAT and FAAH-1; residues comprising the catalytic triad of FAAH-1 (Lys¹⁴², Ser²¹⁷ and nucleophile Ser²⁴¹) are highlighted. (B) Model of rat FLAT (left) based on the structure of FAAH-ΔTM (right), a FAAH-1 mutant lacking the al helix (Alexander, J. P. et al., Chem Biol, 12, 1179 (2005)) (redrawn in green for illustration purposes). Most of the al helix and the entire α2 helix (orange) of FAAH-1 are absent in FLAT. Both variants contain a membrane-binding domain (blue, FLAT residues 343-367) and an ‘α2-interacting loop’ (red, FLAT residues 187-210), which may interact with the α2 helix and help shield the enzyme's catalytic pocket from water. The membrane model was generated using Molecular Dynamics simulations of a 1,2-dioleoyl-sn-glycerol-3-phosphorylcholine bilayer (Rosso, L. et al., J Comput Chem, 29, 24 (2008)).

FIG. 2. Identification of FLAT mRNA in rat and mouse tissues. (A-B) Reverse transcriptase-polymerase chain reaction analyses show the presence of the FLAT transcript (bottom band) in various brain regions and tissues of the rat. The top band is the FAAH-1 transcript. Both bands were identified by nucleotide sequencing. (C) Southern blot analyses of cDNA generated by reverse transcription of total RNA extracted from rat brain (1) or liver (2). We hybridized blots with radioactive probes complementary to either the 3′- or 5′-terminus of FAAH-1. The 3′-probe recognized only one band, corresponding to FAAH-1 cDNA, whereas the 5′-probe recognized two bands, corresponding to FAAH-1 cDNA (top) and FLAT cDNA (bottom). (D) Ribonuclease protection assays showing the presence of FLAT (left panel) and FAAH-1 (right panel) mRNA in rat brain and liver. (1) Radioactive probe digested with T1 ribonuclease; (2) Undigested probe; (3) T1 ribonuclease digestion after hybridization with rat liver mRNA; (4) T1 ribonuclease digestion after hybridization with rat brain mRNA. Top arrows indicate the position of the radioactive probe; bottom arrows indicate the position of the hybridized product. (E) Western blot analysis of protein extracts from liver (1) or brain (2) of wild-type C57/B16 mice (wt) or FAAH-1-deficient mice (FAAH^{-/-}). Numbers on the left of the gel indicate molecular weight markers (in kD). The apparent molecular weights of FAAH-1 (65 kD) and FLAT (56 kD) are shown on the right.

FIG. 3. Hydrolysis of various radioactively labeled lipids by extracts of vector-transfected HEK293 cells (open bars), FLAT-expressing HEK293 cells (closed bars) or FAAH-1-expressing HEK293 cells (shaded bars). Hydrolysis of (A) [³H]-anandamide; (B) [³H]-oleoylethanolamide (OEA); (C) [³H]-2-oleoyl-sn-glycerol (2-AG). Amidase and esterase activities were assayed as described in Supplementary Methods. Results are expressed as mean±SEM of 5-6 separate experiments. ***, P<0.001 versus vector control, ANOVA followed by Dunnett's test.

FIG. 4. Enhanced flexibility of the α2-interacting loop may increase access of water to the substrate-binding pocket of FLAT. The bottom panel shows changes in average water coordination of the side-chain nitrogen in Lys¹⁴² (upper line, left y axis) and the χ1 dihedral value of Met¹⁹¹ (lower line, right y axis) as a function of simulated time. Met¹⁹¹ participates in substrate binding by accepting on its backbone carbonyl a hydrogen bond from the amide nitrogen of anandamide. Furthermore, its side chain may gate the access of water through the cytosolic channel to the catalytic pocket. Gray areas in the plot highlight two different regimes of water coordination; the last configuration in each regime is displayed in the top panels, which show a detail of the catalytic site of FLAT (white ribbons) together with the α2 helix (transparent right ribbons) of FAAH-ΔTM, after superposition of the common core of the two proteins. The α2-interacting loop is shown in violet. The catalytic triad (Ser²¹⁷, Lys¹⁴² and nucleophile Ser²⁴¹) and Met¹⁹¹ are shown as a licorice model. For each configuration, the overall displacement from the starting structure (corresponding to the end of the thermalization) of flexible residues belonging to the α2-interacting loop and the catalytic region is shown with arrows, with the length of each arrow proportional to the calculated displacement. The solvation in proximity of Lys¹⁴² is shown by using transparent blue isocontour surfaces representing the average number of water molecules in each window (˜1 in the left window, ˜3 in the right window). The blue sphere in the right window symbolizes a water molecule that has a high probability of being found in that position.

FIG. 5. Electrostatic potential differences between FLAT and FAAH-1. Superimposition of representative structures taken from Molecular Dynamics simulations of FAAH-ΔTM and FLAT (white ribbons). The α2 helix of FAAH-ΔTM is shown on the right and a clipping plane passing through catalytic triad residue Lys¹⁴² is represented and colored according to the difference between the electrostatic potential generated by FLAT and FAAH-ΔTM. Right and left color codes of the bottom bar correspond to positive and negative potential differences, respectively. Lys¹⁴² is located in an area that displays an overall negative potential difference.

FIG. 6. FLAT binds to anandamide and facilitates its accumulation in cells. (A) Specific binding of [³H]-anandamide to rat FLAT-glutathione-S-transferase (GST) (closed squares) or GST alone (open squares). The inset shows a Scatchard transformation (bound−[bound/free], in nmol) of binding data. (B) AM404 (closed squares) antagonizes [³H]-anandamide binding to FLAT-GST, whereas URB597 (open circles) has no effect. (C) Cytosolic fractions of FLAT-expressing Hek-293 cells contain detectable amounts of FLAT (arrow); a corresponding fraction from FAAH-1-expressing cells is shown for comparison. Results are from one experiment, replicated twice with identical results. (D) [³H]-Anandamide accumulation in control (open bars) or FLAT-expressing Hek-293 cells (closed bars) incubated with vehicle (dimethylsulfoxide, final concentration 0.01%), AM404 or non-radioactive anandamide (μM). (E) The anandamide transport inhibitors UCM-707 and VDM-11 inhibit [³H]-anandamide accumulation in FAAH-1-expressing Hek-293 cells. This process is not affected by URB597 or mutation of catalytic Ser²⁴² (shaded bar). (F) Accumulation of radioactively labeled lipids into control (open bars) or FLAT-expressing Hek-293 cells (closed bars). Abbreviations: [³H]-oleoylethanolamide, OEA; [³H]-palmitoylethanolamide, PEA; [³H]-arachidonic acid, AA; [³H]-2-arachidonoyl-sn-glycerol, 2-AG. Results are expressed as the mean±SEM of 3-7 experiments. ***P<0.01, versus vector-transfected cells, Student's t test; ^{#}P<0.05; ^{##}, P<0.01; ^{###}, P<0.001 versus vehicle, one-way ANOVA followed by Dunnett's test.

FIG. 7. Subcellular localization of recombinant FLAT and FAAH-1 in extracts of Hek293 cells. (A) We fractionated cell extracts by ultracentrifugation and subjected the fractions to Western blot analyses using antibodies for V5 (top) and β-actin (bottom): (1) cytosolic fraction; (2) membrane fraction. After treating membranes with Na₂CO₃ (0.1 M), we separated soluble (3) and insoluble (4) protein fractions and analyzed them by Western blot. Bands were visualized by electrochemiluminescence. (B) We quantified band intensities using the National Institutes of Health Image software, with β-actin as an internal standard.

FIG. 8. Funnel plot summarizing the stepwise nature of the virtual ligand-screening process. Compounds were selected from the Molcart database (Molsoft, LaJolla, Calif.). At each stage, an increasingly severe filtering procedure was applied to prioritize compounds while preserving chemical diversity.

FIG. 9. ARN272 antagonizes [³H]-anandamide binding to FLAT and prevents anandamide transport. (A) Effects of ARN272 on [³H]-anandamide binding to FLAT-GST. The inset shows the chemical structure of ARN272. (B) Effects of ARN272 (μM) on [³H]-anandamide accumulation in FLAT-expressing Hek-293 cells (closed bars); the open bar represents vector-transfected cells. (C) Effects of vehicle (open bar), ARN272 and AM404 (closed bars) on [³H]-anandamide accumulation in rat cortical neurons in cultures. (D) Effects of ARN272 and URB597 on FAAH activity in rat brain membranes. (E-F) Effects of ARN272 (1 mg per kg of body weight, intraperitoneal) on plasma levels of anandamide, OEA, PEA and 2-AG in mice. Results are the mean±SEM of 3-7 experiments. ***, P<0.001 versus vector-transfected cells, Student's t test; #, P<0.05 and ##, P<0.01 versus vehicle; one-way ANOVA followed by Dunnett's test.

FIG. 10. ARN272 produces CB₁ receptor-dependent analgesia. We elicited a local inflammation in mice by intraplantar injection of carrageenan (car) and measured pain behaviors and edema immediately (0 h) and 2 h after injection. ARN272 (mg per kg of body weight, intraperitoneal) decreased (A) mechanical hyperalgesia (withdrawal threshold, in seconds), (B) thermal hyperalgesia (withdrawal latency, in seconds), and (C) edema (volume, in ml). The CB₁ antagonist AM251 (1 mg per kg of body weight, intraperitoneal) abolished the effects of ARN272 on (D) mechanical hyperalgesia, (E) thermal hyperalgesia, and (F) edema. Results are the mean±SEM of 6 mice per group. *, P<0.05; **, P<0.01; ***, P<0.001 versus vehicle-injected controls; two-way ANOVA followed by Bonferroni's test.

FIG. 11. Antinociceptive effects of ARN272 4 h after intraplantar carrageenan injection. Administration of ARN272 (mg per kg of body weight, intraperitoneal) reduced carrageenan-induced (A) mechanical hyperalgesia (withdrawal threshold, in seconds), (B) thermal hyperalgesia (withdrawal latency, in seconds), and (C) paw edema (volume, in ml). Results are expressed as the mean±SEM of 6 mice per group. ***, P<0.001 versus control; two-way ANOVA followed by Bonferroni's test.

FIG. 12. The CB₂ receptor antagonist AM630 and the transient receptor potential vanilloid-1 (TRP-V1) antagonist AMG9810 (each at 1 mg per kg of body weight, intraperitoneal, 30 min before carrageenan) did not alter the antinociceptive effects of ARN272 (1 mg per kg of body weight, intraperitoneal, injected together with carrageenan) on (A) mechanical hyperalgesia, (B) thermal hyperalgesia, and (C) paw edema. Results are expressed as the mean±SEM of 6 mice per group.

FIG. 13. ARN272 does not productively interact with CB₁ cannabinoid receptors. Effects of vehicle or ARN272 (in μM) on [³H]-CP55940 binding rat brain membranes. Binding is significantly inhibited by methyl arachidonyl fluorophosphonate (MAFP). *, P<0.05 and ***, P<0.001 versus vector control, n=4.

### DETAILED DESCRIPTION OF THE INVENTION

The invention relates to the Applicants discovery that FLAT is the molecule effects membrane transport of anandamide in vivo and that inhibitors of FLAT can modulate the activity and effects of endogenous anandamide in vivo. The invention also relates to the Applicants's structural and functional characterization of the FLAT molecule and the use of that characterization and the molecule itself to identify other inhibitors of FLAT having therapeutic potential based upon the modulation of anandamide in vivo.

The molecular entity involved in anandamide translocation is still unknown and the mechanistic bases of this process remain controversial (Hillard, C. J. et al., J Mol Neurosci, 33, 18 (2007); Glaser, S. T. et al., Proc Natl Acad Sci USA, 100, 4269 (2003)). The present invention provides a partly cytosolic variant of FAAH-1, present in the rodent brain and termed FLAT, which lacks amidase activity, but binds anandamide with low micromolar affinity and confers anandamide transport to cells that are engineered to express it. AM404 and other anandamide transport inhibitors suppress these effects. Moreover, we disclose a small-molecule competitive inhibitor of the interaction of anandamide with FLAT, ARN272, and show that this agent suppresses anandamide internalization in vitro and interrupts anandamide deactivation in vivo. Our results identify FLAT as a key component of the transport system that internalizes anandamide in neural cells.

The functional properties of FLAT disclosed herein closely match those previously attributed to the presumptive carrier responsible for anandamide internalization by neurons and astrocytes in cultures (Beltramo, M. et al., Science, 277, 1094 (1997); Hillard, C. J. et al., J Neurochem, 69, 631 (1997); Hillard, C. J. et al., J Mol Neurosci, 33, 18 (2007)). Our studies show indeed that FLAT selectively binds to and internalizes anandamide, and that three known inhibitors of anandamide translocation—the substrate mimics AM404, VDM-11 and UCM-707 (Di Marzo, V. et al., Nat Rev Drug Discov, 7, 438 (2008))—prevent these effects. Moreover, we show that a small-molecule competitive inhibitor of the interaction of anandamide with FLAT, the phthalazine derivative ARN272, suppresses anandamide accumulation by rat brain neurons in vitro and reproduces key pharmacological effects of transport blockade in vivo, including analgesia in a mouse model of inflammatory pain (Beltramo, M. et al., Science, 277, 1094 (1997)). Consistent with the present results, prior work has shown that deletion of Faah-1 gene substantially reduces anandamide transport in mouse cortical neurons, whereas acute blockade of FAAH activity fails to do so (Ortega-Gutierrez, S. et al., Biochemistry, 43, 8184 (2004); Kathuria, S. et al., Nat Med, 9, 76 (2003)). Our findings identify FLAT as a critical component of anandamide transport in neural cells and a potential molecular target for therapeutic drugs. Notably, we found that expression of FLAT does not confer [3H]-2-AG transport to Hek293 cells, and administration of the FLAT inhibitor ARN272 does not increase plasma levels of 2-AG in mice. These data indicate that FLAT does not contribute to 2-AG translocation in neural cells (Beltramo, M. et al., Neuroreport, 11, 1231 (2000)) and raise the possibility that other endocannabinoid carriers remain to be discovered. The observation that faah-1 deletion reduces, but does not entirely abolish anandamide internalization supports this possibility (Ortega-Gutierrez, S. et al., Biochemistry, 43, 8184 (2004)).

In a first aspect, accordingly, the invention provides pharmaceutical compositions comprising a therapeutically effective amount of a compound for use according to the invention. These compounds are compounds of Formula I:

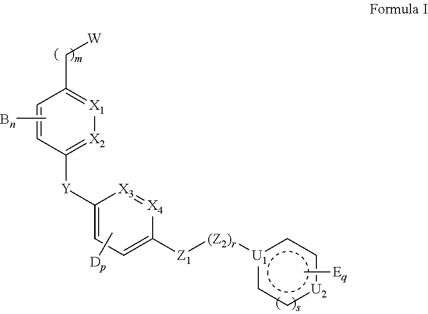

wherein W is aryl, heteroaryl, heterocycloalkyl, or alkyl, wherein the aryl, heteroaryl, or heterocycloalkyl can be substituted by 1 to 3 substituents selected from lower alkyl, alkenyl, OH, alkoxy, cyano, halogen, NR¹R², NR¹COR², CONR¹R², wherein R¹ and R² are independently selected from H or lower alkyl; m is an integer from 0 to 1; X₁, X₂, X₃, and X₄ are independently selected from carbon and nitrogen; n is an integer from 0 to 2; each B member is independently selected from the group consisting of alkoxy, alkyl, alkenyl, halogen and heteroalkyl, and, optionally, when n is 2 and two B members are on adjacent carbon atoms, the two adjacent B members may be taken together along with the atoms to which they are attached to form a saturated or unsaturated ring comprising 5 to 6 ring atoms; Y is independently selected from O, NR³, and C═O, wherein R³ is hydrogen or lower alkyl; p is an integer from 0 to 4; each D member is independently selected from the group consisting of alkoxy, alkyl, alkenyl, halogen, and heteroalkyl, and optionally when two D members are on adjacent carbon atoms, the adjacent D members may be taken together along with the atoms to which they are attached to form a saturated or unsaturated ring comprising 5 to 6 ring atoms; Z₁ and Z₂ are independently selected from C═O and NR⁴, wherein R⁴ is hydrogen or lower alkyl; r is an integer from 0 to 1; s is an integer from 0 to 1; U₁ is independently selected from C or N; U₂ is independently selected from C, O, and N(R⁵)ₜ, wherein the ring containing U₁ and U₂ can be aromatic, or partially or fully saturated, R⁵ is hydrogen or lower alkyl, and t is 0 or 1, with the proviso that when the ring containing U₁ and U₂ is aromatic, U₁ is C and t is 0; q is an integer from 0 to 4; each E member is independently selected from the group consisting of alkoxy, alkyl, alkenyl, halogen, and heteroalkyl, OH, cyano, NR⁶R², wherein R⁶ and R⁷ are hydrogen or lower alkyl, and optionally when two E members are on adjacent carbon atoms the adjacent E members may be taken together along with the atoms to which they are attached to form a saturated or unsaturated ring comprising 5 to 6 ring atoms.

In a particularly preferred embodiment, the compound is ARN272 or a pharmaceutically acceptable salt thereof

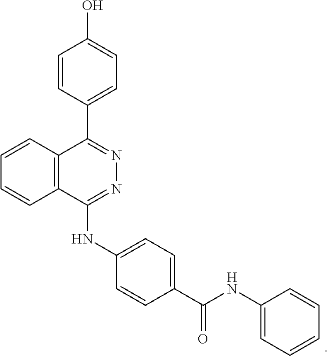 [SMILES: O=C(Nc1ccccc1)c1ccc(Nc2nnc(-c3ccc(O)cc3)c3ccccc23)cc1]

### ARN272 (4-{[4-(4-hydroxyphenyl)phthalazin-1-yl]amino-N-phenylbenzamide}

ARN272 represents a new rigid scaffold, pharmacophore for FLAT which, as a rigid scaffold, can be readily modified to improve its properties using standard structure-activity relationship studies.

Certain compounds of Formula I are preferred for use according to the invention. With respect to Formula I, the compounds belong to the following embodiments as outlined:

(A) Preferred are embodiments (A) (a to g), wherein compounds for use according to the invention are selected from those of formula I according to any of the following limitations (a to g):
a. W is phenyl and m is 0;
b. Z₁ is C═O and Z₂ is NR⁴
c. X₁ and X₂ are N and X₃ and X₄ are C;
d. Y is NR³;
e. (a) and (b) as immediately above
f. (a) and (b) and (c) as immediately above.
g. (a) and (b) and (c) and (d) as immediately above.
   (B) Further preferred are embodiments (B)(a-g) wherein the compounds of Formula I are selected according to any of the following limitations (a to g):
a. W is unsubstituted or with 1 or 2 substituents selected from hydroxy, alkyl, or halo, alkoxy, or cyano;
b. U₁ and U₂ are C,
c. R⁴ is H,
d. r is 1,
e. (a) and (b) as immediately above,
f. (a) and (b) and (c) as immediately above, or
g. (a) and (b) and (c) and (d) as immediately above.
   Additionally preferred are those compounds which satisfy any of the limitations of both any of (A)(a-g) above and any of (B)(a-g) above.
   (C). Further preferred are embodiments (C)(a-g) wherein the compounds of Formula I are selected according to any of the following limitations a to g:
a. s is 1;
b. p is 0 or 1,
c. n is 0, 1, or 2;
d. m is 0 or 1;
e. (a) and (b) as immediately above,
f. (a) and (b) and (c) as immediately above, or
g. (a) and (b) and (c) and (d) as immediately above.
   Additionally preferred are those compounds which satisfy any of the limitations of both any of (A)(a-g) above and any of (C)(a-g) above; both of any of (B)(a-g) above and any of (C)(a-g) above; and each of (A)(a-g), (B)(a-g) and (C)(a-g) above.
   (D) Further preferred are embodiments (D)(a-g) wherein the compounds of Formula I are selected according to any of the following limitations a-h:
a. n is 2 and two B members on adjacent carbon atoms are taken together to form a six membered saturated ring,
b. n is 2 and two B members on adjacent carbon atoms are taken together to form a six membered unsaturated ring,
c. p is 0;
d. m is 0;
e. q is 0; or
f. (a) and (b) as immediately above,
g. (a) and (b) and (c) as immediately above, or
h. (a) and (b) and (c) and (d) as immediately above.
   Additionally preferred are those compounds which satisfy any of the limitations of both any of (A)(a-g) above and any of (D)(a-g) above; of both any of (B)(a-g) above and any of (D)(a-g) above; of both any of (C)(a-g) above and any of (D)(a-g); of both any of (A)(a-g)(B)(a-g) above and any of (D)(a-g); of both (A)(a-g)(B)(a-g)(C)(a-g) above and any of (D)(a-g) above; of both any of (A)(a-g)(C)(a-g) above and any of (D)(a-g).
   (E) Further preferred are embodiments (E)(a-f) wherein the compounds of formula I are selected according to any of the following limitations a-g:
a. R³ is H;
b. W is substituted with hydroxy or halo or alkyl;
c. D is alkyl or halo;
e. (a) and (b) as immediately above, or
f. (a) and (b) and (c) as immediately above,
   Additionally preferred are those compounds which satisfy any of the limitations of both any of (A)(a-g) above and any of (E)(a-f) above; of both any of (B)(a-g) above and any of (E)(a-f) above; of both any of (C)(a-g) above and any of (E)(a-f); of both any of (A)(a-g)(B)(a-g) above and any of (E)(a-f); of both (A)(a-g)(B)(a-g)(C)(a-g) above and any of (E)(a-f); of both any of (A)(a-g)(C)(a-g) above and any of (E)(a-f) above.
   Additionally preferred are those compounds which satisfy any of the limitations of both any of (A)(a-g)(D)(a-g) and any of (E)(a-f) above; of both any of (B)(a-g)(D)(a-g) above and any of (E)(a-f) above; of both any of (C)(a-g)(D)(a-g) and any of (E)(a-f) above; of both any of (A)(a-g)(B)(a-g)(D)(a-g) and any of (E)(a-f) above; of both (A)(a-g)(B)(a-g)(C)(a-g)(D)(a-g) and any of (E)(a-f) above; of both any of (A)(a-g)(C)(a-g)(D)(a-g) and any of (E)(a-f) above.

F. Further preferred are embodiments of (F)(a-e), wherein
a. each alkyl, alkoxy, alkylene, heteroalkyl substituent of Formula I, if present, is from 1 to 3 carbon atoms (e.g., a methyl, methoxy, ethyl, ethoxy, ethylene, propyl, propoxy, propylene);
b. each alkyl, alkoxy, alkylene, heteroalkyl substituent of Formula I, if present, has from 1 to 6 carbon atoms
c. each lower alkyl of Formula I, if present, is a C1 to C3 alkyl (e.g., methyl, ethyl, propyl).
d. (a) and (c) as immediately above; or
e. (b) and (c) as immediately above.
   Additionally preferred are those compounds which satisfy any of the limitations of both any of (A)(a-g) (E)(a-f) above and (F)(a-e) above; of both any of (B)(a-g)(E)(a-f) above and (F)(a-e) above; of both any of (C)(a-g)(E)(a-f) and (F)(a-e) above; of both any of (A)(a-g) (B)(a-g)(E)(a-f) and (F)(a-e) above; of both (A)(a-g)(B)(a-g)(C)(a-g)(E)(a-f) and (F)(a-e) above; of both any of (A)(a-g)(C)(a-g)(E)(a-f) and (F)(a-e) above; additionally preferred are those compounds which satisfy any of the limitations of both any of (A)(a-g)(D)(a-g)(E)(a-f) above and (F)(a-e) above; of both any of (B)(a-g)(D)(a-g)(E)(a-f) above and (F)(a-e) above; of both any of (C)(a-g)(D)(a-g)(E)(a-f) above and (F)(a-e) above; of both any of (A)(a-g) (B)(a-g)(D)(a-g)(E)(a-f) above and (F)(a-e) above; of both (A)(a-g)(B)(a-g)(C)(a-g)(D)(a-g) (E)(a-f) above and (F)(a-e) above; of both any of (A)(a-g)(C)(a-g)(D)(a-g)(E)(a-f) above and (F)(a-e) above.

For instance, the compound of A(g)B(g)C(g)(Dg) and E(g) would represent a compound where W is phenyl substituted with hydroxy, halo or alkyl, m is 0; Z₁ is C═O and Z₂ is NH, X₁ and X₂ are N and X₃ and X₄ are C; U₁ and U₂ are C, r is 1, s is 1, Y is NH, and p, q and m are each 0.

With regard to any of the above combinations and subcombinations, a limitation of a subcombination which is inconsistent with any preceding combination or subcombination is treated as absent with respect to the specific combinations and subcombinations with which it would conflict. Accordingly, in this example, in the case where D(g) provides a preceding combination or subcombination, the E(c) and E(f) limitations would be treated as absent specifically with respect to any combinations or subcombinations made with D(g).

In another aspect the invention provides compounds for use in the compositions and methods of treatment the invention. The compounds for use according to the invention generally may contain one or more asymmetric centers and can thus occur as racemates and racemic mixtures, single enantiomers, diastereomeric mixtures and individual diastereomers. The present invention is meant to comprehend all such isomeric forms of the inventive compounds. The compounds for use according to the invention include the diastereoisomers of pairs of enantiomers. Diastereomers for example, can be obtained by fractional crystallization from a suitable solvent, for example methanol or ethyl acetate or a mixture thereof. The pair of enantiomers thus obtained may be separated into individual stereoisomers by conventional means, for example by the use of an optically active acid as a resolving agent. Alternatively, any enantiomer of such a compound of the invention may be obtained by stereospecific synthesis using optically pure starting materials of known configuration. The compounds of the present invention may have unnatural ratios of atomic isotopes at one or more of their atoms. For example, the compounds may be radiolabeled with isotopes, such as tritium or carbon-14. All isotopic variations of the compounds of the present invention, whether radioactive or not, are within the scope of the present invention.

The subject compounds may be isolated in the form of their pharmaceutically acceptable acid addition salts, such as the salts derived from using inorganic and organic acids. Such acids may include hydrochloric, nitric, sulfuric, phosphoric, formic, acetic, trifluoroacetic, propionic, maleic, succinic, malonic and the like. In addition, certain compounds containing an acidic function can be in the form of their inorganic salt in which the counterion can be selected from sodium, potassium, lithium, calcium, magnesium and the like, as well as from organic bases. The term “pharmaceutically acceptable salts” refers to salts prepared from pharmaceutically acceptable non-toxic bases or acids including inorganic bases or acids and organic bases or acids.

The invention also encompasses prodrugs of the present compounds, which on administration undergo chemical conversion by metabolic processes before becoming active pharmacological substances. In general, such prodrugs will be derivatives of the present compounds that are readily convertible in vivo into a functional compound of the invention. Conventional procedures for the selection and preparation of suitable prodrug derivatives are described, for example, in “Design of Prodrugs”, ed. H. Bundgaard, Elsevier, 1985. The invention also encompasses active metabolites of the present compounds.

Some of the compounds described herein contain olefinic double bonds, and unless specified otherwise, are meant to include both E and Z geometric isomers.

Some of the compounds described herein may exist with different points of attachment of hydrogen, referred to as tautomers. Such an example may be a ketone and its enol form known as keto-enol tautomers. The individual tautomers as well as mixture thereof are encompassed by the inventive Formulas.

### DEFINITIONS

Unless otherwise defined, all technical and scientific terms used herein have the same meaning as commonly understood by one of ordinary skill in the art to which this invention belongs. Each publication, patent application, patent, and other reference cited herein is incorporated by reference in its entirety to the extent that it is not inconsistent with the present disclosure.

Unless otherwise stated, the following terms used in the specification and claims are defined for the purposes of this Application and have the following meanings. It is noted here that as used in this specification and the appended claims, the singular forms “a,” “an,” and “the” include plural reference unless the context clearly dictates otherwise.

“FAAH” denotes a mammalian Fatty Acid Amide Hydrolase and includes, but is not limited to, the human, rat, and mouse forms of the enzyme. U.S. Pat. No. 6,271,015 discloses isolated and purified forms of FAAH. In one set of embodiments, the FAAH IC₅₀ of the subject compounds is defined according to inhibition of the rat enzyme under physiologically relevant conditions. FAAHs are enzymes responsible for the degradation of lipid ethanolamides, (Fowler, C. J. et al., Biochem. Pharmacol. 62, 517-526 (2001); Patricelli, M. P. et al. Vitam. Horm., 62, 663-674 (2001)) e.g. anandamide, (Devane, W. A. et al., Science 258, 1946-1949 (1992)) oleoylethanolamide, (Rodríguez de Fonseca, F. et al. Nature (London) 414, 209-212 (2001); Fu, J. et al., Nature (London) 425, 90-93 (2003)) and palmitoylethanolamide, (Calignano, A. et al. Nature (London) 394, 277-281 (1998); Lambert, D. M. et al., Curr. Med. Chem. 9, 663-674 (2002)) a biochemical process which, along with selective trasport into cells in the case of anandamide, (Di Marzo, V., Nature (London) 372, 686-691 (1994); Beltrama, M. et al., Science 277, 1094-1097 (1997); Piomelli, D. et al., Proc. Natl. Acad. Sci. U.S.A. (2002)) brings about the cessation of the cellular effects of these autacoids. The human FAAH1 protein and nucleic acid sequences and crystalline structures are known (see, NCBI database accession no. 000519, version 000519.2 GI:60416391 which is incorporated by reference in its entirety with respect to the sequences disclosed therein)

“FLAT” or “FAAH-1b” denotes a catalytically silent, partly cytosolic FAAH variant that drives the membrane transport of anandamide. FLAT lacks amidase activity but binds anandamide with low micromolar affinity and facilitates its transport into cells. The sequence of human cDNA FLAT is set forth in SEQ ID NO:1. A nucleic acid sequence encoding human FLAT and the FLAT amino acid sequence is shown in SEQ ID NO:2. A preferred FLAT for use according to the invention is an isolated FLAT protein or recombinant FLAT protein. In some embodiments, an isolated FLAT protein is a protein composition having at least 10%, 20%, 50% or 75%, or 90% of the protein or of the amandamide-binding protein in the composition as FLAT.

The term “contacting” includes reference to placement in direct physical association.

The invention also provides an “isolated” FLAT nucleic acid or protein which refers to a FLAT tnucleic acid or protein, respectively, which is no longer in the natural environment from which it was isolated, e.g., no longer associated with other materials present in the particular organism from which it was first obtained in nature.

An “expression plasmid” comprises a nucleotide sequence encoding a molecule or interest, which is operably linked to a promoter.

As used herein, “polypeptide”, “peptide” and “protein” are used interchangeably and include reference to a polymer of amino acid residues. The terms apply to amino acid polymers in which one or more amino acid residue is an artificial chemical analogue of a corresponding naturally occurring amino acid, as well as to naturally occurring amino acid polymers. The terms also apply to polymers containing conservative amino acid substitutions such that the protein remains functional.

“Inhibitors,” “activators,” and “modulators” of expression or of activity are used to refer to inhibitory, activating, or modulating molecules, respectively, identified using in vitro and in vivo assays for expression or activity, e.g., ligands, agonists, antagonists, and their homologs and mimetics. The term “modulator” includes inhibitors and activators. Inhibitors are agents that, e.g., inhibit expression of a polypeptide or polynucleotide of the invention or bind to, partially or totally block stimulation or enzymatic activity, decrease, prevent, delay activation, inactivate, desensitize, or down regulate the activity of a polypeptide or polynucleotide of the invention, e.g., antagonists. Activators are agents that, e.g., induce or activate the expression of a polypeptide or polynucleotide of the invention or bind to, stimulate, increase, open, activate, facilitate, enhance activation or enzymatic activity, sensitize or up regulate the activity of a polypeptide or polynucleotide of the invention, e.g., agonists. Modulators include naturally occurring and synthetic ligands, antagonists, agonists, small chemical molecules and the like. Assays to identify inhibitors and activators include, e.g., applying putative modulator compounds to cells, in the presence or absence of a polypeptide or polynucleotide of the invention and then determining the functional effects on a polypeptide or polynucleotide of the invention activity. Samples or assays comprising a polypeptide or polynucleotide of the invention that are treated with a potential activator, inhibitor, or modulator are compared to control samples without the inhibitor, activator, or modulator to examine the extent of effect. Control samples (untreated with modulators) are assigned a relative activity value of 100% Inhibition is achieved when the activity value of a polypeptide or polynucleotide of the invention relative to the control is, for example, about 80%, optionally 50% or 25-1%. Activation is achieved when the activity value of a polypeptide or polynucleotide of the invention relative to the control is 110%, optionally 150%, optionally 200-500%, or 1000-3000% higher.

The term “test compound” or “drug candidate” or “modulator” or grammatical equivalents as used herein describes any molecule, either naturally occurring or synthetic, e.g., protein, oligopeptide (e.g., from about 5 to about 25 amino acids in length, preferably from about 10 to 20 or 12 to 18 amino acids in length, preferably 12, 15, or 18 amino acids in length), small organic molecule, polysaccharide, lipid, fatty acid, polynucleotide, RNAi, siRNA, antibody, oligonucleotide, etc. The test compound can be in the form of a library of test compounds, such as a combinatorial or randomized library that provides a sufficient range of diversity. Test compounds are optionally linked to a fusion partner, e.g., targeting compounds, rescue compounds, dimerization compounds, stabilizing compounds, addressable compounds, and other functional moieties. Conventionally, new chemical entities with useful properties are generated by identifying a test compound (called a “lead compound”) with some desirable property or activity, e.g., inhibiting activity, creating variants of the lead compound, and evaluating the property and activity of those variant compounds. Often, high throughput screening (HTS) methods are employed for such an analysis. Preferred compounds for testing include those embraced by the compounds for use according to the invention.

Construction of suitable vectors containing the desired gene coding FLAT and control sequences employs standard ligation and restriction techniques, which are well understood in the art (see Maniatis et al., in Molecular Cloning: A Laboratory Manual, Cold Spring Harbor Laboratory, New York (1982)). Isolated plasmids, DNA sequences, or synthesized oligonucleotides are cleaved, tailored, and re-ligated in the form desired.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are near each other, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

“Determining the functional effect” refers to assaying for a compound that increases or decreases a parameter that is indirectly or directly under the influence of a polynucleotide or polypeptide of the invention, e.g., measuring physical and chemical or phenotypic effects. Such functional effects can be measured by any means known to those skilled in the art, e.g., changes in spectroscopic (e.g., fluorescence, absorbance, refractive index), hydrodynamic (e.g., shape), chromatographic, or solubility properties for the protein; measuring inducible markers or transcriptional activation of the protein; measuring binding activity or binding assays, e.g. binding to antibodies; measuring changes in ligand binding affinity; measurement of calcium influx; measurement of the accumulation of an enzymatic product of a polypeptide of the invention or depletion of an substrate; changes in enzymatic activity, e.g., kinase activity, measurement of changes in protein levels of a polypeptide of the invention; measurement of RNA stability; G-protein binding; GPCR phosphorylation or dephosphorylation; signal transduction, e.g., receptor-ligand interactions, second messenger concentrations (e.g., cAMP, IP3, or intracellular Ca2+); identification of downstream or reporter gene expression (CAT, luciferase, β-gal, GFP and the like), e.g., via chemiluminescence, fluorescence, colorimetric reactions, antibody binding, inducible markers, and ligand binding assays.

Samples or assays comprising a nucleic acid or protein disclosed herein that are treated with a potential activator, inhibitor, or modulator are compared to control samples without the inhibitor, activator, or modulator to examine the extent of inhibition. Control samples (untreated with inhibitors) are assigned a relative protein activity value of 100%. Inhibition is achieved, for example, when the activity value relative to the control is about 80%, preferably 50%, more preferably 25-0%. Activation is achieved when the activity value relative to the control (untreated with activators) is 110%, more preferably 150%, more preferably 200-500% (i.e., two to five fold higher relative to the control), more preferably 1000-3000% higher.

A “small organic molecule” refers to an organic molecule, either naturally occurring or synthetic, that has a molecular weight of more than about 50 Daltons and less than about 2500 Daltons, preferably less than about 2000 Daltons, preferably between about 100 to about 1000 Daltons, more preferably between about 200 to about 500 Daltons.

As used herein, “recombinant” includes reference to a protein produced using cells that do not have, in their native state, an endogenous copy of the DNA able to express the protein. The cells produce the recombinant protein because they have been genetically altered by the introduction of the appropriate isolated nucleic acid sequence. The term also includes reference to a cell, or nucleic acid, or vector, that has been modified by the introduction of a heterologous nucleic acid or the alteration of a native nucleic acid to a form not native to that cell, or that the cell is derived from a cell so modified. Thus, for example, recombinant cells express genes that are not found within the native (non-recombinant) form of the cell, express mutants of genes that are found within the native form, or express native genes that are otherwise abnormally expressed, under expressed or not expressed at all.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

As used herein, “nucleic acid” or “nucleic acid sequence” includes reference to a deoxyribonucleotide or ribonucleotide polymer in either single- or double-stranded form, and unless otherwise limited, encompasses known analogues of natural nucleotides that hybridize to nucleic acids in a manner similar to naturally occurring nucleotides. Unless otherwise indicated, a particular nucleic acid sequence includes the complementary sequence thereof as well as conservative variants, i.e., nucleic acids present in wobble positions of codons and variants that, when translated into a protein, result in a conservative substitution of an amino acid.

As used herein, “encoding” with respect to a specified nucleic acid, includes reference to nucleic acids which comprise the information for translation into the specified protein. The information is specified by the use of codons. Typically, the amino acid sequence is encoded by the nucleic acid using the “universal” genetic code. However, variants of the universal code, such as is present in some plant, animal, and fungal mitochondria, the bacterium Mycoplasma capricolum (Proc. Nat'l Acad. Sci. USA 82:2306-2309 (1985), or the ciliate Macronucleus, may be used when the nucleic acid is expressed in using the translational machinery of these organisms.

As used herein, “expressed” includes reference to translation of a nucleic acid into a protein. Proteins may be expressed and remain intracellular, become a component of the cell surface membrane or be secreted into the extracellular matrix or medium.

By “host cell” is meant a cell which can support the replication or expression of the expression vector. Host cells may be prokaryotic cells such as E. colit, or eukaryotic cells such as yeast, insect, amphibian, or mammalian cells, including human cells.

In the present description and in the claims, “appetency disorders” are understood as meaning disorders associated with a substance and especially abuse of a substance and/or dependency on a substance, disorders of food behaviors, especially those liable to cause excess weight, irrespective of its origin, for example: bulimia, appetency for sugars, non-insulin-dependent diabetes. Appetizing substances are therefore understood as meaning substances to be taken into the body and for which an appetite or craving for such consumption is present by any route of entry or self-administration. Appetizing substances includes, foods, and their appetizing ingredients such as sugars, carbohydrates, or fats, as well as drinking alcohol or drugs of abuse or excess consumption. An “appetite’ may be directed toward such substances as foods, sugars, carbohydrates, fats, as well as ethanol or drugs of abuse or addiction or excess consumption (e.g., tobacco, CNS depressants, CNS stimulants).

Appetite refers to the desire to consume an appetizing substance or the behavior of consuming appetizing substances. An appetizing substance may be a food or sugar or other substance. In one embodiment, the appetizing substance is a food. In some embodiments, the appetizing substance is a drug of abuse such as ethanol, nicotine, cocaine, an opioid, a CNS stimulant or a CNS depressant.

Anxiety is a state of fearfulness which is unprovoked by an environmental threat or highly disproportionate to an environmental threat. Anxiety may be acute and short term lasting hours to days; or chronic and lasting from many days to weeks or longer.

The term clinical anxiety refers to any form of anxiety for which treatment is necessary or indicated in order to alleviate it. Such clinical anxiety may be persistent or recurrent and typically severe. Anxiety disorders include, but are not limited to, any of the anxiety disorders as provided in the Diagnostic and Statistical Manual of Mental Disorders, Fourth Edition. (Copyright 1994 American Psychiatric Association) which is hereby incorporated by reference. Such disorders include, but are not limited to, panic disorder, agoraphobia, generalized anxiety disorder, specific phobia, social phobia, obsessive-compulsive disorder, acute stress disorder, and post-traumatic stress disorder; and adjustment disorders with anxious features, anxiety disorders due to general medical conditions, substance-induced anxiety disorders, and the residual category of anxiety disorder not otherwise specified.

Depressive disorders and conditions include, but are not limited to, any of the depressive disorders and conditions as provided in the Diagnostic and Statistical Manual of Mental Disorders, Fourth Edition (Copyright 1994 American Psychiatric Association). These disorders include major depressive disorder (unipolar depression), dysthymic disorder (chronic, mild depression), and bipolar disorder (manic-depression). Clinical depression refers to any form of depression that requires some form of treatment in order to alleviate it. Such clinical depression may persist for months and last for most of every day and seriously impairs the quality of life.

A “major depressive episode” is defined as at least two weeks of depressed mood or loss of interest, which may be accompanied by other symptoms of depression. The symptoms must persist for most of the day (i.e. for at least two thirds of the patients' waking hours), nearly every day (i.e. for at least ten out of fourteen days) for at least two consecutive weeks. A “depressed mood” is often described by the patient as feeling sad, hopeless, helpless or worthless. The patient may also appear sad to an observer, for example, through facial expression, posture, voice and tearfulness. In children and adolescents, the mood may be irritable. A “loss of interest” is often described by the patient as feeling less interested in hobbies or not feeling any enjoyment in activities that were previously considered to be pleasurable. A major depressive episode may be accompanied by other symptoms of depression including significant weight loss when not dieting or weight gain (e.g. a change of more than 5% body weight in one month), or decrease or increase in appetite; insomnia or hypersomnia; psychomotor agitation or retardation; fatigue or loss of energy; feelings of worthlessness or excessive or inappropriate guilt; diminished ability to think or concentrate; or indecisiveness; and recurrent thoughts of death, recurrent suicidal ideation with or without a specific plan, or a suicide attempt.

Schizophrenia and related disorders include, but are not limited to the following types: Catatonic Type; Disorganized Type; Paranoid Type; Residual Type; Undifferentiated Type; and Schizophreniform Disorder as provided in the Diagnostic and Statistical Manual of Mental Disorders, Fourth Edition. TEXT REVISION Copyright 2000 American Psychiatric Association which is hereby incorporated be reference.

The term “effective amount” means a dosage sufficient to produce a desired result with respect to the indicated disorder, condition, or mental state. The desired result may comprise a subjective or objective improvement in the recipient of the dosage. With respect to anxiety, the improvement may be decreased sign or symptom of anxiety.

The terms “treatment”, “therapy” and the like include, but are not limited to, methods and manipulations to produce beneficial changes in a recipient's status. The changes can be either subjective or objective and can relate to features such as symptoms or signs of the disease, disorder or condition being treated. For example, if the patient notes decreased fearfulness, anxiety or worry, then successful treatment of anxiety or an anxiety disorder has occurred. For example, if a decrease in the frequency or severity of pain or inflammation is noted, then a beneficial treatment of the pain or inflammation has occurred. For example, if depressive ideation is reduced, a beneficial change in depression or a depressive disorder has been achieved. Similarly, if the clinician notes objective changes, such as decreases in tremulousness or agitation, then treatment for anxiety has also been beneficial or successful. Preventing the deterioration of a recipient's status is also included by the term. Therapeutic benefit includes any of a number of subjective or objective factors indicating a response of the condition being treated as discussed herein.

“Therapeutically-effective amount” refers to the amount of an active agent sufficient to induce a desired biological or clinical result. That result may be alleviation of the signs, symptoms, or causes of a disease, or any other desired alteration of a biological system. The term “therapeutically effective amount” is used herein to denote any amount of the formulation which causes a substantial improvement in a disease, disorder or condition when administered to a subject over a period of time. The amount will vary with the condition being treated, the stage of advancement of the condition, and the type and concentration of formulation applied. Appropriate amounts in any given instance will be readily apparent to those skilled in the art or capable of determination by routine experimentation.

“Alkyl” represented by itself means a straight or branched, saturated aliphatic radical containing one to eight carbon atoms, unless otherwise indicated e.g., alkyl includes methyl, ethyl, propyl, isopropyl, butyl, sec-butyl, isobutyl, tert-butyl, n-pentyl, n-hexyl, n-heptyl, n-octyl and the like. The structural isomers of these groups (e.g., sec-butyl, isobutyl, tert-butyl for butyl), are also contemplated. As part of another substituent, “alkyl” means, unless otherwise stated, a straight or branched chain, saturated, hydrocarbon radical, having the number of carbon atoms designated (i.e. (C₁-C₆) means one to six carbons). “Lower alkyl” is preferably an alkyl having from 1 to 3 carbon atoms.

As used herein, the term “alkoxy” represents an alkyl moiety joined to the remainder of the molecule by the oxygen atom of the alkoxy. Accordingly, examples of alkoxy would include, but not be limited to, methoxy, ethoxy, propoxy and the like.

The term “alkenyl” is derived from the name of the corresponding alkyl group but differs in possessing one or more double bonds. Similarly, “alkynyl” groups are named with respect to their corresponding alkyl group but differs in possessing one or more triple bonds. Non-limiting examples of such unsaturated alkenyl groups and alkynyl groups include vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers.

“Alkylene”, unless indicated otherwise, means a straight or branched, saturated aliphatic, divalent radical having the number of one to six carbon atoms, e.g., methylene (—CH₂—), ethylene (—CH₂CH₂—), trimethylene (—CH₂CH₂CH₂—), tetramethylene (—CH₂CH₂CH₂CH₂—)2-methyltetramethylene(—CH₂CH(CH₃)CH₂CH₂—), pentamethylene (—CH₂CH₂CH₂CH₂CH₂—), and the like.

“Alkoxyalkyl” means a linear monovalent hydrocarbon radical of one to six carbon atoms or a branched monovalent hydrocarbon radical of three to six carbons substituted with at least one alkoxy group, preferably one or two alkoxy groups, as defined above, e.g., 2-methoxy-ethyl, 1-, 2-, or 3-methoxypropyl, 2-ethoxyethyl, and the like.

“Aromatic” refers to a moiety wherein the constituent atoms make up an unsaturated ring system, all atoms in the ring system are sp² hybridized and the total number of pi electrons is equal to 4n+2.

The term “aryl” means, unless otherwise stated, an aromatic, hydrocarbon substituent which can be a single ring or multiple rings (preferably from 1 to 3 rings) which are fused together or linked covalently. The term “heteroaryl” refers to aryl groups (or rings) that contain from one to four heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl.

As used herein, the term “halogen” or “halo” refers to iodine (I), bromine (Br), chlorine (Cl), and/or fluorine (F). Fluoro and chloro are particularly preferred.

“Haloalkyl” refers to alkyl as defined above substituted by one or more, for example from one to thirteen, preferably from one to seven, “halo” atoms, as such terms are defined in this Application. Haloalkyl includes monohaloalkyl, dihaloalkyl, trihaloalkyl, perhaloalkyl and the like e.g. chloromethyl, dichloromethyl, difluoromethyl, trifluoromethyl, 2,2,2-trifluoroethyl, perfluoroethyl, 2,2,2-trifluoro-1,1-dichloroethyl, and the like.

“Haloalkylene” means alkylene radical as defined above wherein one to four, preferably one or two hydrogen atoms in the alkylene chain has(have) been replaced by fluorine atom(s).

“Hydroxy” means —OH radical. Unless indicated otherwise, the compounds of the invention containing hydroxy radicals include protected derivatives thereof. Suitable protecting groups for hydroxy moieties include benzyl and the like.

“Hydroxyalkyl” means a linear monovalent hydrocarbon radical of one to six carbon atoms or a branched monovalent hydrocarbon radical of three to six carbons substituted with one or two hydroxy groups, provided that if two hydroxy groups are present they are not both on the same carbon atom. Representative examples include, but are not limited to, hydroxymethyl, 2-hydroxyethyl, 2-hydroxypropyl, 3-hydroxypropyl, 1-(hydroxymethyl)-2-methylpropyl, 2-hydroxybutyl, 3-hydroxybutyl, 4-hydroxybutyl, 2,3-dihydroxypropyl, 1-(hydroxymethyl)-2-hydroxyethyl, 2,3-dihydroxybutyl, 3,4-dihydroxybutyl and 2-(hydroxymethyl)-3-hydroxypropyl, preferably 2-hydroxyethyl, 2,3-dihydroxypropyl, and 1-(hydroxymethyl)-2-hydroxyethyl.

As used herein, the term “heteroalkyl” derives its name from the corresponding alkyl group but differs in containing one, two, or three heteroatoms independently selected from N, O, and S each substituting for a carbon of an alkyl group. The heteroatom nitrogen and sulfur atoms are optionally oxidized, and the nitrogen atom(s) are optionally quaternized. A heteroalkyl group is attached to the remainder of the molecule through a carbon atom of the heteroalkyl group and the heteroatoms of the heteroalkyl are not contiguous with another heteroatom.

The term “heteroalkenyl” derives its name from the corresponding alkenyl group but differs in having 1, 2, or 3 heteroatoms substituting for a carbon of the alkenyl group. The heteroatom nitrogen and sulfur atoms are optionally oxidized, and the nitrogen atom(s) are optionally quaternized. A heteroatom can form a double bond with a carbon atom. A heteroalkenyl group is attached to the remainder of the molecule through a carbon atom of the hydrocarbyl and the heteroatoms of the hydrocarbyl are not contiguous with another heteroatom.

As used herein, the term “cycloalkyl” refers to a saturated monocyclic hydrocarbon radical comprising from about 3 to about 8 carbon atoms, and more preferably 3 to 6 carbon atoms. The term “cycloalkenyl” refers to monocyclic, non-aromatic hydrocarbon radical comprising from about 5 to about 6 carbon atoms and having at least one double bond. Exemplary cycloalkyl groups and cycloalkenyl groups include cyclopropyl, cyclobutyl, cyclopentyl, cyclohexyl, cyclohexenyl, cyclohepta-1,3-dienyl, and the like.

As used herein, the term “heterocycloalkyl” refers to a saturated or partially unsaturated monocyclic hydrocarbon radical comprising from about 3 to about 8 carbon atoms, and more preferably 3 to 6 carbon atoms in which 1, 2 or 3 of the carbon atoms are independently replaced by a heteroatom independently selected from O, N, or S, Nitrogen and sulfur atoms are optionally oxidized, and the nitrogen atom(s) are optionally quaternized. Sulfur maybe in the thio, sulfinyl or sulfonyl oxidation state. The term “heterocycloalkenyl” refers to heterocycloalkyl group having at least one double bond. A heterocycloalkyl or heterocycloalkenyl group is attached to the remainder of the molecule through a carbon atom, respectively, of the heterocycloalkyl or heterocycloalkenyl group; and the heteroatoms of the heterocycloalkyl or heterocycloalkenyl are not contiguous with another heteroatom of the heterocycloalkyl or heterocycloalkenyl.

As used herein, the term “heteroatom” is meant to include oxygen (O), nitrogen (N), and sulfur (S)).

“Isomers” mean compounds of Formula (I) having identical molecular formulae but differ in the nature or sequence of bonding of their atoms or in the arrangement of their atoms in space. Isomers that differ in the arrangement of their atoms in space are termed “stereoisomers”. Stereoisomers that are not mirror images of one another are termed “diastereomers” and stereoisomers that are nonsuperimposable mirror images are termed “enantiomers” or sometimes “optical isomers”. A carbon atom bonded to four nonidentical substituents is termed a “chiral center”. A compound with one chiral center that has two enantiomeric forms of opposite chirality is termed a “racemic mixture”. A compound that has more than one chiral center has 2ⁿ⁻¹ enantiomeric pairs, where n is the number of chiral centers. Compounds with more than one chiral center may exist as either an individual diastereomer or as a mixture of diastereomers, termed a “diastereomeric mixture”. When one chiral center is present a stereoisomer may be characterized by the absolute configuration of that chiral center. Absolute configuration refers to the arrangement in space of the substituents attached to the chiral center. Enantiomers are characterized by the absolute configuration of their chiral centers and described by the R- and S-sequencing rules of Cahn, Ingold and Prelog. Conventions for stereochemical nomenclature, methods for the determination of stereochemistry and the separation of stereoisomers are well known in the art (e.g., see “Advanced Organic Chemistry”, 4th edition, March, Jerry, John Wiley & Sons, New York, 1992). It is understood that the names and illustration used in this Application to describe compounds of Formula (I) are meant to be encompassed all possible stereoisomers.

Compounds of the invention include any diastereoisomers or pairs of any enantiomers. Diastereomers for example, can be obtained by fractional crystallization from a suitable solvent, for example methanol or ethyl acetate or a mixture thereof. The pair of enantiomers thus obtained may be separated into individual stereoisomers by conventional means, for example by the use of an optically active acid as a resolving agent.

Alternatively, any enantiomer of such a compound of the invention may be obtained by stereospecific synthesis using optically pure starting materials of known configuration.

The compounds of the present invention may have unnatural ratios of atomic isotopes at one or more of their atoms. For example, the compounds may be radiolabeled with isotopes, such as tritium or carbon-14. All isotopic variations of the compounds of the present invention, whether radioactive or not, are within the scope of the present invention.

“Optional” or “optionally” or “may be” means that the subsequently described event or circumstance may or may not occur, and that the description includes instances where the event or circumstance occurs and instances in which it does not.

“Pharmaceutically acceptable” means that which is useful in preparing a pharmaceutical composition that is generally safe, and neither biologically nor otherwise undesirable and includes that which is acceptable for veterinary use as well as human pharmaceutical use.

“Pharmaceutically acceptable salts” means salts of compounds of Formula (I) which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as acetic acid, propionic acid, hexanoic acid, heptanoic acid, cyclopentanepropionic acid, glycolic acid, pyruvic acid, lactic acid, malonic acid, succinic acid, malic acid, maleic acid, fumaric acid, tartaric acid, citric acid, benzoic acid, o-(4-hydroxybenzoyl)benzoic acid, cinnamic acid, mandelic acid, methylsulfonic acid, ethanesulfonic acid, 1,2-ethanedisulfonic acid, 2-hydroxy-ethanesulfonic acid, benzenesulfonic acid, p-chlorobenzenesulfonic acid, 2-naphthalenesulfonic acid, p-toluenesulfonic acid, camphorsulfonic acid, 4-methylbicyclo[2.2.2]oct-2-ene-1-carboxylic acid, glucoheptonic acid, 4,4′-methylenebis(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid and the like.

Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like.

The term “pharmaceutically acceptable carrier” encompasses any of the standard pharmaceutical carriers, buffers and excipients, including phosphate-buffered saline solution, water, and emulsions (such as an oil/water or water/oil emulsion), and various types of wetting agents and/or adjuvants. Suitable pharmaceutical carriers and their formulations are described in Remington's Pharmaceutical Sciences (Mack Publishing Co., Easton, 19th ed. 1995). Preferred pharmaceutical carriers depend upon the intended mode of administration of the active agent. Typical modes of administration are described below.

The term “subject” as used herein includes any animal, including, but not limited to, mammals (e.g., rat, mouse, cat, dog) including humans to which a treatment is to be given. “Mammal” includes humans and non-human mammals (e.g., dogs, cats, rabbits, cattle, horses, sheep, goats, swine, rats, mice, and primates).

The term “effective amount” means a dosage sufficient to produce a desired result with respect to the indicated disorder, condition, or mental state. The desired result may comprise a subjective or objective improvement in the recipient of the dosage. With respect to pain, the improvement may be decreased sign or symptom of pain.

The terms “treatment”, “therapy” and the like include, but are not limited to, methods and manipulations to produce beneficial changes in a recipient's health status. The changes can be either subjective or objective and can relate to features such as symptoms or signs of the disease, disorder or condition being treated. For example, if the patient notes decreased pain, then successful treatment of pain has occurred. For example, if a decrease in the amount o swelling has occurred, then a beneficial treatment of inflammation has occurred. Similarly, if the clinician notes objective changes, such as improved range of motion, then treatment for a pain or inflammation which had been impairing the motion has also been beneficial. Preventing the deterioration of a recipient's status is also included by the term. Therapeutic benefit includes any of a number of subjective or objective factors indicating a beneficial response or improvement of the condition being treated as discussed herein.

“Pharmaceutically-acceptable” or “therapeutically-acceptable” refers to a substance which does not interfere with the effectiveness or the biological activity of the active ingredients and which is not toxic to the hosts in the amounts used, and which hosts may be either humans or animals to which it is to be administered.

“Disease” specifically includes any unhealthy condition of an animal or human or part thereof and includes an unhealthy condition that may be caused by, or incident to, medical or veterinary therapy applied to that animal, i.e., the “side effects” of such therapy.

“Therapeutically-effective amount” refers to the amount of an active agent sufficient to induce a desired biological or clinical result. That result may be alleviation of the signs, symptoms, or causes of a disease, or any other desired alteration of a biological system. The term “therapeutically effective amount” is used herein to denote any amount of the formulation which causes a substantial improvement in a disease, disorder or condition when administered to a subject. The amount will vary with the condition being treated, the stage of advancement of the condition, and the type and concentration of formulation applied. Appropriate amounts in any given instance will be readily apparent to those skilled in the art or capable of determination by routine experimentation.

A “prophylactic treatment” is a treatment administered to a subject who does not exhibit signs of a neurological or psychological disorder or condition or exhibits only early or slight signs of such a disorder or condition, wherein treatment is administered for the purpose of decreasing the risk of developing a pathology or worsening of disorder or condition. The compounds of the invention may be given as a prophylactic treatment to prevent undesirable or unwanted anxiety or panic attacks, or to reduce the level of anxiety should worsening occur.

### Recombinant Methods

### Methods for Preparation of FLAT Gene Constructs, Proteins, and Recombinant Cells

Methods for recombinant production of nucleic acid sequences and polypeptide sequences are well known to one of ordinary skill in the art. For instance, the gene of interest can be amplified from RNA from animal tissue via reverse-transcriptase PCR. Following ligation in an appropriate vector, the gene constructs can be used for transfection of mammalian cell lines or transformation of prokaryotic strains for overexpression. Cells containing the FLAT gene constructs may express full-length FLAT, truncated protein fragments, or FLAT fusion proteins. The fusion proteins may containing affinity handles, such as glutathione S-transferase segments, for ease in purification.

The FLAT nucleic acid sequences of the present invention can be prepared by any suitable method including, for example, cloning of appropriate sequences or by direct chemical synthesis by methods such as the phosphotriester method of Narang et al., Meth. Enzymol. 68:90-99 (1979); the phosphodiester method of Brown et al., Meth. Enzymol. 68:109-151 (1979); the diethylphosphoramidite method of Beaucage et al., Tetra. Lett. 22:1859-1862 (1981); the solid phase phosphoramidite triester method described by Beaucage & Caruthers, Tetra. Letts. 22(20):1859-1862 (1981), e.g., using an automated synthesizer as described in, for example, Needham-VanDevanter et al. Nucl. Acids Res. 12:6159-6168 (1984); and, the solid support method of U.S. Pat. No. 4,458,066. Chemical synthesis produces a single stranded oligonucleotide. This may be converted into double stranded DNA by hybridization with a complementary sequence, or by polymerization with a DNA polymerase using the single strand as a template. One of skill would recognize that while chemical synthesis of DNA is limited to sequences of about 100 bases, longer sequences may be obtained by the ligation of shorter sequences.

The FLAT nucleic acid sequences of this invention and proteins can be prepared by cloning techniques. Examples of appropriate cloning and sequencing techniques, and instructions sufficient to direct persons of skill through many cloning exercises are found in Sambrook et al., MOLECULAR CLONING: A LABORATORY MANUAL (2ND ED.), Vols. 1-3, Cold Spring Harbor Laboratory (1989)), Berger and Kimmel (eds.), GUIDE TO MOLECULAR CLONING TECHNIQUES, Academic Press, Inc., San Diego Calif. (1987)), or Ausubel et al. (eds.), CURRENT PROTOCOLS IN MOLECULAR BIOLOGY, Greene Publishing and Wiley-Interscience, NY (1987). Product information from manufacturers of biological reagents and experimental equipment also provide useful information. Such manufacturers include the SIGMA chemical company (Saint Louis, Mo.), R&D systems (Minneapolis, Minn.), Pharmacia LKB Biotechnology (Piscataway, N.J.), CLONTECH Laboratories, Inc. (Palo Alto, Calif.), Chem Genes Corp., Aldrich Chemical Company (Milwaukee, Wis.), Glen Research, Inc., GIBCO BRL Life Technologies, Inc. (Gaithersberg, Md.), Fluka Chemica-Biochemika Analytika (Fluka Chemie AG, Buchs, Switzerland), Invitrogen, San Diego, Calif., and Applied Biosystems (Foster City, Calif.), as well as many other commercial sources known to one of skill.

Once the nucleic acids encoding a FLAT are isolated and cloned, one may express the desired protein in a recombinantly engineered cell such as bacteria, yeast, insect (especially employing baculoviral vectors), and mammalian cells. A “recombinant protein” is a protein produced using cells that do not have in their native form an endogenous copy of the DNA able to express the protein. The cells produce the recombinant protein because they have been genetically altered by the introduction of the appropriate isolated nucleic acid-sequence (e.g., a vector comprising an FLAT nucleic acid).

It is expected that those of skill in the art are knowledgeable in the numerous expression systems available for expression of DNA encoding FLAT proteins. No attempt to describe in detail the various methods known for the expression of proteins in prokaryotes or eukaryotes will be made. In brief summary, the expression of natural or synthetic nucleic acids encoding the FLAT proteins of the present invention will typically be achieved by operably linking the DNA or cDNA to a promoter (which is either constitutive or inducible), followed by incorporation into an expression vector. The vectors can be suitable for replication and integration in either prokaryotes or eukaryotes. Typical expression vectors contain transcription and translation terminators, initiation sequences, and promoters useful for regulation of the expression of the DNA encoding the FLAT protein. To obtain high level expression of a cloned gene, it is desirable to construct expression vectors which contain, at the minimum, a strong promoter to direct transcription, a ribosome binding site for translational initiation, and a transcription/translation terminator. One of skill would recognize that modifications can be made to an FLAT protein without diminishing its biological activity. Some modifications may be made to facilitate the cloning, expression, or incorporation of the targeting molecule into a fusion protein. Such modifications are well known to those of skill in the art and include, for example, a methionine added at the amino terminus to provide an initiation site, or additional amino acids (e.g., poly His) placed on either terminus to create conveniently located restriction sites or termination codons or purification sequences.

### Expression in Prokaryotes

Examples of regulatory regions suitable for this purpose in E. coli are the promoter and operator region of the E. coli tryptophan biosynthetic pathway as described by Yanofsky, Bacteriol. 158:1018-1024 (1984), and the leftward promoter of phage lambda (P.sub.L) as described by Herskowitz and Hagen, Ann. Rev. Genet., 14:399-445 (1980). The inclusion of selection markers in DNA vectors transfected in E. coli is also useful. Examples of such markers include genes specifying resistance to ampicillin, tetracycline, or chloramphenicol. See, Sambrook et al. for details concerning selection markers for use in E. coli. The vector is selected to allow introduction into the appropriate host cell. Bacterial vectors are typically of plasmid or phage origin. Appropriate bacterial cells are infected with phage vector particles or transfected with naked phage vector DNA. If a plasmid vector is used, the bacterial cells are transfected with the plasmid vector DNA. Expression systems for expressing FLAT proteins are available using E. coli, Bacillus sp. and Salmonella (Palva et al., Gene, 22:229-235 (1983); Mosbach et al., Nature 302:543-545 (1983)).

Detection of the expressed protein is achieved by methods known in the art and include, for example, radioimmunoassays, Western blotting techniques or immunoprecipitation. Purification from E. coli can be achieved following procedures described in U.S. Pat. No. 4,511,503. A variety of eukaryotic expression systems such as yeast, insect cell lines, bird, fish, frog, and mammalian cells, are known to those of skill in the art. As explained briefly below, FLAT proteins of the present invention may be expressed in these eukaryotic systems.

### Methods of Assaying Compounds for FLAT Activity.

### I. Assays for Modulators of FLAT

Modulation of FLAT, and corresponding modulation of cellular transport of anandamide can be assessed using a variety of in vitro and in vivo assays, including cell-based models. Such assays can be used to test for inhibitors and activators of a FLAT, and, consequently, inhibitors and activators of neuronal activity, pain and inflammation and the other conditions for treatment according to the invention.

Measurement of FLAT activity can be conducted in a cell expressing FLAT, either recombinant or naturally occurring, can be performed using a variety of assays, in vitro, in vivo, and ex vivo, as described herein. A suitable physical, chemical or phenotypic change that affects activity, e.g., enzymatic activity such as anandamide transport or response, kinase activity, pr r ligand binding can be used to assess the influence of a test compound on the polypeptide of this invention. When the functional effects are determined using intact cells or animals, one can also measure a variety of effects, such as, ligand binding, kinase activity, transcriptional changes to both known and uncharacterized genetic markers (e.g., northern blots), changes in cell metabolism, changes related to nerve cell activity.

A. In Vitro Assays

Assays to identify compounds with FLAT modulating activity can be performed in vitro. Purified recombinant or naturally occurring FLAT can be used in the in vitro methods of the invention. As described below, the binding assay can be either solid state or soluble. Preferably, the protein or membrane is bound to a solid support, either covalently or non-covalently. Often, the in vitro assays of the invention are substrate or ligand binding or affinity assays, either non-competitive or competitive. Other in vitro assays include measuring changes in spectroscopic (e.g., fluorescence, absorbance, refractive index), hydrodynamic (e.g., shape), chromatographic, or solubility properties for the protein. Other in vitro assays include enzymatic activity assays, such as phosphorylation or autophosphorylation assays).

In one embodiment, a high throughput binding assay is performed in which the FLAT protein or a fragment thereof is contacted with a potential modulator and incubated for a suitable amount of time. In one embodiment, the potential modulator is bound to a solid support, and the FLAT protein is added. In another embodiment, the FLAT protein is bound to a solid support. A wide variety of modulators can be used, as described below, including small organic molecules, peptides, antibodies, and FLAT ligand analogs. A wide variety of assays can be used to identify FLAT-modulator binding, including labeled protein-protein binding assays, electrophoretic mobility shifts, immunoassays, enzymatic assays such as kinase assays, and the like. In some cases, the binding of the candidate modulator is determined through the use of competitive binding assays, where interference with binding of a known ligand or substrate is measured in the presence of a potential modulator.

In one embodiment, microtiter plates are first coated with FLAT and then exposed to one or more test compounds potentially capable of inhibiting the binding of FLAT to a known ligand. In a binding competition assay, a labeled (i.e., fluorescent, enzymatic, radioactive isotope) binding partner of the FLAT is used as a competing ligand and inhibition of the labeled ligand binding indicates the test compound binds to FLAT. The results are compared to a control sample that was not exposed to a test compound, which exhibits uninhibited signal.

B. Cell-Based In Vivo Assays

In another embodiment, FLAT protein is expressed in a cell, and functional, e.g., physical and chemical or phenotypic, changes are assayed to identify FLAT modulators. Cells expressing FLAT proteins can also be used in binding assays and enzymatic assays. Any suitable functional effect can be measured, as described herein. For example, cellular morphology (e.g., cell volume, nuclear volume, cell perimeter, and nuclear perimeter), ligand binding, kinase activity. The FLAT protein can be naturally occurring or recombinant.

Cellular FLAT polypeptide levels can be determined by measuring the level of protein or mRNA. The level of FLAT protein or proteins related to FLAT are measured using immunoassays such as western blotting, ELISA and the like with an antibody that selectively binds, respectively, to the FLAT polypeptide or a fragment thereof. For measurement of mRNA, amplification, e.g., using PCR, LCR, or hybridization assays, e.g., northern hybridization, RNAse protection, dot blotting, are preferred. The level of protein or mRNA is detected using directly or indirectly labeled detection agents, e.g., fluorescently or radioactively labeled nucleic acids, radioactively or enzymatically labeled antibodies, and the like, as described herein.

Alternatively, FLAT expression can be measured using a reporter gene system. Such a system can be devised using an FLAT protein promoter operably linked to a reporter gene such as chloramphenicol acetyltransferase, firefly luciferase, bacterial luciferase, β-galactosidase and alkaline phosphatase. Furthermore, the protein of interest can be used as an indirect reporter via attachment to a second reporter such as red or green fluorescent protein (see, e.g., Mistili & Spector, Nature Biotechnology, 15:961-964 (1997)). The reporter construct is typically transfected into a cell. After treatment with a potential modulator, the amount of reporter gene transcription, translation, or activity is measured according to standard techniques known to those of skill in the art.

### —FLAT Binding Activity

Small molecule ligands of FLAT can be selected from large libraries via simulation experiments, via in vitro or in vivo assay, or via a combination of techniques. Small molecules with affinity for FLAT can be selected via a virtual screening process that accounts for the physico-chemical properties of the compounds, degree of similarity to other known FAAH binders, and simulated docking to protein models.

The assays for compounds described herein are amenable to high throughput screening. Preferred assays thus detect binding of the inhibitor to FAAH or the release of a reaction product (e.g., fatty acid amide or ethanolamine) produced by the hydrolysis of a substrate such as oleoylethanolamide or anandamide. The substrate may be labeled to facilitate detection of the released reaction products. High throughput assays for the presence, absence, or quantification of particular reaction products are well known to those of skill in the art. Thus, for example, U.S. Pat. No. 5,559,410 discloses high throughput screening methods for proteins, and U.S. Pat. No. 5,576,220 and U.S. Pat. No. 5,541,061 disclose high throughput methods of screening for ligand/antibody binding.

In addition, high throughput screening systems are commercially available (see, e.g., Zymark Corp., Hopkinton, Mass.; Air Technical Industries, Mentor, Ohio; Beckman Instruments, Inc. Fullerton, Calif.; Precision Systems, Inc., Natick, Mass., etc.). These systems typically automate entire procedures including all sample and reagent pipetting, liquid dispensing, timed incubations, and final readings of the microplate in detector(s) appropriate for the assay. These configurable systems provide high throughput and rapid start up as well as a high degree of flexibility and customization. The manufacturers of such systems provide detailed protocols the various high throughput. Thus, for example, Zymark Corp. provides technical bulletins describing screening systems for detecting the modulation of gene transcription, ligand binding, and the like.

### —Anandamide Transport Inhibition Assay Methods

Assays for anandamine transport inhibition are well known to one of ordinary skill in the art. Exemplary methods for screening such compounds and identifying novel suitable compounds with such inhibitory activity are taught in U.S. Patent Application Publication No. 20040048907 published on Mar. 11, 2004 (U.S. Patent Serial No. 439347, filed May 15, 2003), PCT Patent Publication No. WO 03/097573, and U.S. Patent Application Publication No. 20030149082. Such assays can be used to identify other anandamide transport inhibitors for use according to the present invention. Exemplary anandamide transport inhibitors for use according to the invention include M404, AM1172, OMDM1 and UCM707. U.S. Patent Application Publication No. 20040048907 and PCT Patent Publication No. WO 03/097573 are herein incorporated by reference in their entirety and in particular with respect to the anandamide transport inhibitors and anandamide transport inhibition assays disclosed therein. The anandamide transport inhibitors are known in the art. See U.S. Patent Application Publication No. 20030149082 published on Aug. 7, 2003 (application Ser. No. 09/328,742), which is herein incorporated by reference in its entirety and in particular with respect to the anandamide transport inhibition assays disclosed therein and the therapeutic uses and formulations of such inhibitors.

### Methods for Screening Compounds for a Therapeutic Activity

### —Antinociceptive Activity.

Methods for screening compositions for an antinociceptive effect are known to one of ordinary skill in the art. For instance, the test compounds can be administered to the subject animals in the mouse hot-plate test and the mouse formalin test and the nociceptive reactions to thermal or chemical tissue damage measured. See also U.S. Pat. No. 6,326,156 which teaches methods of screening for antinociceptive activity. See Cravatt et al. Proc. Natl. Acad. Sci. U.S.A. 98:9371-9376 (2001). In addition, methods of screening endocannabinoid transport inhibitor in the treatment of neuropathic and inflammatory pain are known (see, La Rana G, et al., J Pharmacol Exp Ther 317(3) pp. 1365-71 (2006) which is incorporated by reference with respect to such screening methods):

Formalin test. Swiss mice are acclimateed in the testing room for at least 12 h beforehand. Formalin (5% formaldehyde in sterile saline, 10 μl) is injected into the plantar surface of the left hind paw using a 27-gauge needle fitted to a microsyringe and the mice are immediately transferred to a transparent observation chamber. Nocifensive behavior (licking and biting of the injected paw) is monitored continuously for a period of about 45 min divided into two intervals: phase I, 0-15 min; and phase II, 15-45 min (see, Dubuisson D and Dennis SG Pain 4: 161-174 (1977)).

CCI Model. The sciatic nerve of Wistar rats is surgically ligated as described previously (Bennett G J and Xie Y K. Pain 33: 87-107 (1988)) and then the animals are anesthetized with ketamine (100 mg/kg i.p) and xylazine (5 mg/kg i.p.). The left sciatic nerve is exposed at midthigh level through a small incision, and one-third to one-half of the nerve thickness is loosely ligated using silk thread. The wound is closed with muscle suture and skin clips and dusted with streptomycin powder. As a control, in sham-operated animals, the nerve is similarly exposed but not ligated. Behavioral tests are performed on the day before surgery (day −1) and again about 1 and two weeks after surgery.

CFA Test.

Complete Freund's adjuvant (CFA) is administered in a vehicle of paraffin oil/mannide mono-oleate (85:15, v/v; 0.1 ml) by intradermal injection into the left hind paw of Wistar rats using a 27-gauge needle fitted to a microsyringe on 3 separate days (1, 3, and 7) (see, Billingham M E J, in Anti-Rheumatic Drugs (Orme MCL'E, ed) pp. 1-47 (1990), Pergamon Press, New York). Behavioral tests are performed before the first CFA injection (day −1) and again on day 14 of treatment.
Mechanical Hyperalgesia. Mechanical hyperalgesia can be measured using a Randall-Selitto analgesimeter (Ugo Basile, Varese, Italy). Latencies of paw withdrawal in response to a calibrated pressure are assessed on both ligated and controlateral paws before and after ligation or CFA treatment and later again at 1 and two weeks. The cut-off force can be set at about 150 g.
Thermal Hyperalgesia can be measured using a Hargreaves apparatus (Hargreaves K, et al., Pain 32: 77-88 (1988). (Ugo Basile). Two days before the experiment, the animals are placed in a transparent Perspex box with a thin glass floor and allowed to acclimate for 10 to 15 min. A focused beam of radiant heat is applied to the plantar surface and latencies of paw withdrawal are assessed on both ligated and controlateral paws on day −1 (before ligation or CFA treatment) and again on days 7 and 14. Cut-off time is set at 1 min.
Rotorod Test. Integrity of motor function can be assessed in CCI rats using an accelerating Rotorod (Ugo Basile). The animals are acclimated to acceleration in three training runs. Mean performance time (seconds) determined on the fourth and fifth runs served as control value. Performance time is measured every 20 min for a total of 80 min on days 7 and 14 after surgery.

### —Screening for Anxiolytic Activity

One of ordinary skill in the art would appreciate that there are a number of animal models available for assessing the antianxiety effects of a compound. Two pharmacologically validated animal models of anxiety are the elevated zero maze test, and the isolation-induced ultrasonic emission test. The zero maze consists of an elevated annular platform with two open and two closed quadrants and is based on the conflict between an animal's instinct to explore its environment and its fear of open spaces, where it may be attacked by predators (Bickerdike, M. J. et al., Eur. J. Pharmacol., 271, 403-411 (1994); Shepherd, J. K. et al., Psychopharmacology, 116, 56-64 (1994)). Clinically used anxiolytic drugs, such as the benzodiazepines, increase the proportion of time spent in, and the number of entries made into, the open compartments.

A second test for an antianxiety compound is the ultrasonic vocalization emission model, which measures the number of stress-induced vocalizations emitted by rat pups removed from their nest (Insel, T. R. et al., Pharmacol. Biochem. Behay., 24, 1263-1267 (1986); Miczek, K. A. et al., Psychopharmacology, 121, 38-56 (1995); Winslow, J. T. et al., Biol. Psychiatry, 15, 745-757 (1991).

A large number of animal models have been developed in the attempt to predict the anxiolytic activity of novel compounds in man. Many of these paradigms evaluate animal behavior in a so-called “conflict” situation, i.e. a behavioral response is simultaneously under the influence of two opposing motivational states such as approach and avoidance tendencies. Probably the best known model is the conditioned punishment conflict paradigm in which animals are trained to voluntarily exhibit a certain response (e.g. pressing a lever) in order to receive a reward (e.g. food for a hungry animal). Once the animals exhibit a constant rate of lever-press responding, then short periods are introduced (usually signaled by visual or acoustic signals) during which lever pressing is simultaneously rewarded by food and punished by mild electrical foot shock. Animals exhibit a markedly reduced response rate during these conflict periods, which are also characterized by various overt signs of emotionality. The characteristic effect of benzodiazepine receptor agonists, for example the anxiolytic diazepam, is the disinhibition of punished behavior (resulting in an increase in the rate of responding under punishment) at doses that fail to disrupt unpunished responding. Furthermore, these same active drugs produce an anxiolytic-like effect in the absence of actual punishment, i.e. when the rate of lever pressing is reduced by conditioned fear of punishment. The conflict task does not require conditioned behavioral responses: naive thirsty animals can be offered the opportunity to drink, with drinking punished via contact with an electrified spout. Such punishment-suppressed drinking is disinhibited dose-dependently by benzodiazepine receptor agonists (e.g., diazepam). Exploratory activity can likewise be decreased by contingent punishment and released by treatment with known anxiolytics. Conflict models without punishment are based on the presence of the natural opposing motivational states, on the one hand the tendency to explore and, on the other hand, fear of a novel environment (e.g. dark-light chamber task, elevated plus-maze, consumption of unfamiliar food or normal food in an unfamiliar environment, social interaction between animals unfamiliar with each other). While it is obvious to ascribe the behavioral disinhibitory effect of benzodiazepine receptor agonism in these experimental situations to an anxiolytic-like action, their effect can also be interpreted as a general reduction of the influence of aversive factors or even to an impaired ability to withhold innate or conditioned responses. An anti-frustration effect resulting from benzodiazepine receptor agonism is suggested by the increase of responding which is maintained by response-contingent reward in the situation in which the reward is reduced or omitted. Electrical stimulation of the periaqueductal gray area of the midbrain via chronically implanted electrodes in animals is aversive and elicits a number of emotional reactions; benzodiazepine receptor agonists increase the aversive threshold. States of acute anxiety characterised by behavioral and physiological symptoms (cardiovascular, endocrine) can be induced by chemicals known to be anxiogenic in man, e.g. convulsants such as pentylenetetrazol, inverse agonists at the benzodiazepine receptor agonists administered in subconvulsive doses, or even abrupt drug withdrawal after chronic treatment with high doses of sedatives. Ultrasonic distress cries by rat pups acutely separated from their mothers are decreased by benzodiazepine receptor agonists. See, also, Bortolato M, et al., Neuropsychopharmacology 31(12) pp. 2652-9 (2006) which teaches the use of a number of behavioral tests for assessing the anxiolytic effects for use in assessing an anandamide transport inhibitor and which is incorporated herein by reference in its entirety.

In the open field test, motor behaviors are studied in an opaque open field (100×100×40 cm) as described. The field is illuminated using a ceiling halogen lamp regulated to yield 350 lux at the center of the field. Test subject (rats) are habituated to the field for 10 min the day before testing. On the experimental day, the animals are treated and placed in the center of the field, and locomotor activity (number of lines crossed) and rearing and grooming behavior (number of rearings and time spent in the center of the field) are scored for 5 min at 5, 30, 60, and 120 min after drug administration. Behavior is scored by trained, blinded observers.

Elevated Plus Maze. Adult Wistar rats are placed in a central platform of the test apparatus and observed/video recorded for 5 min in a dim light, sound-attenuated environment. The maze can comprise two open arms (50×10 cm²) and two closed arms (50×10×40 cm³) that extend from a common platform (10×10 cm²) at the center. The apparatus with opaque floor and clear walls is elevated to a height of about 60 cm. Test compounds, positive and negative controls/vehicle are administered about 30 min before testing. The observers are blinded. The percent time spent in open arms, number of head dips and stretched attend postures are measured.

Passive Withdrawal. At about 45 min after an injection of the test compound or a control, adult Wistar rats are placed in a cylindrical stainless-steel chamber about 10 cm in diameter and 20 cm in length) which is open at one end, and placed alongside one of the four walls of an open field (about 90 cm square) and observed or video recorded for 15 min in dim light, sound-attenuated environment. The latency to leave the chamber and the total amount of time spent in the open field are measured by blinded observers.

Isolation-Induced Ultrasonic Vocalizations in 10-day-old Wistar rat pups. Briefly, a single male pup is randomly removed from each litter, weighed, and placed in a shallow glass dish located about 15 cm under a microphone. Vocalizations are recorded to establish a baseline and then for about 15 s about 30 min after administration of the compound or vehicle. The results are typically analyzed as percent change from baseline.

### Screening for Antidepressant Activity

Animal models for depression are also well known to those of ordinary skill in the art. For instance, the effect of the compound of the invention in the treatment of depression can be tested in the model of chronic mild stress induced anhedonia in rats. This model is based on the observation that chronic mild stress causes a gradual decrease in sensitivity to rewards, for example consumption of sucrose, and that this decrease is dose-dependently reversed by chronic treatment with antidepressants. The method has previously been described and more information with respect to the test appears from Willner, Paul, Psychopharmacology, 1997, 134, 319-329.

Another test for antidepressant activity is the forced swimming test (Nature 266, 730-732, 1977) In this test, animals are administered an agent preferably by the intraperitoneal route or by the oral route 30 or 60 minutes before the test. The animals are placed in a crystallizing dish filled with water and the time during which they remain immobile is clocked. The immobility time is then compared with that of the control group treated with distilled water. Imipramine 25 mg/kg. can be used as the positive control. The antidepressant compounds decrease the immobility time of the mice thus immersed.

Another test for antidepressant activity is the caudal suspension test on the mouse (Psychopharmacology, 85, 367-370, 1985) In this test, animals are preferably treated with the study compound by the intraperitoneal route or by the oral route 30 or 60 minutes before the test. The animals are then suspended by the tail and their immobility time is automatically recorded by a computer system. The immobility times are then compared with those of a control group treated with distilled water. Imipramine 25 mg/kg can be used as the positive control. Antidepressant compounds decrease the immobility time of the mice.

Another test for screening antidepressants is the DRL-72 TEST. This test, carried out according to the protocol of Andrews et al [“Effects of imipramine and mirtazapine on operant performance in rats”—Drug Development Research 32, 58-66 (1994)], gives an indication of antidepressant-like activity. See also U.S. Pat. No. 6,403,573.

Additional animal models for screening are well known to one of ordinary skill in the art. For instance, see U.S. Pat. No. 5,952,315.

### —Methods for Assessing the Effect of a Compound on Appetite and Appetency Disorders.

Compounds of the invention can be administered to an animal to determine whether they affect food intake and body weight, body fat, appetite, food seeking behavior, or modulate modulator fatty acid oxidation. Method of conducting such tests are known to one of ordinary skill in the art. For instance, see U.S. Patent Application No. 60/336,289 assigned to the same assignee and herein incorporated by reference in its entirety.

Animals can be, for example, obese or normal guinea pigs, rats, mice, or rabbits. Suitable rats include, for example, Zucker rats. Suitable mice include, for example, normal mice, ALS/LtJ, C3.5W—H-^{2b}/SnJ, (NON/LtJ×NZO/HlJ)F1, NZO/HlJ, ALR/LtJ, NON/LtJ, KK.Cg-AALR/LtJ, NON/LtJ, KK.Cg-A^{y}/J, B6.HRS(BKS)-Cpe^{fat}/+, B6.129P2-Gck^{tm/Efr}, B6.V-Lep^{ab}, BKS.Cg-m+/+Lep^{rd}b, and C57BL/6J with Diet Induced Obesity.

Administration of an appropriate amount the candidate compound may be by any means known in the art such as, for example, oral or rectal, parenteral such as, for example, intraperitoneal, intravenous, subcutaneous, subdermal, intranasal, or intramuscular. Preferably administration may be intraperitoneal or oral. An appropriate effective amount of the candidate compound may be determined empirically as is known in the art.

Methods of assessing appetitive behavior are known to one of ordinary skill in the art. For instance, Maruani et al. (U.S. Pat. No. 6,344,474) teach two such assays. One method of assessing the effect on appetite behavior is to administer a compound to a rat and assess its effect on the intake of a sucrose solution. This method is taught in W. C. Lynch et al., Physiol. Behay., 1993, 54, 877-880. Male Sprague-Dawley rats weighing 190 to 210 g are under a normal light cycle (from 7 am to 7 pm) and receive water and food ad libitum. For 6 days, between 11 am and 3 pm, the food and the water bottles are withdrawn and the rats are given a 5% sucrose solution to drink. Rats drinking less than 3 g of sucrose solution are eliminated. On the seventh day the test is carried out according to the following procedure: 9 am: withdrawal of food, 10 am: administration of the inhibitor or vehicle to the test animals; 11 am=T0: introduction of bottles containing a weighed sucrose solution, T0+1 hour, T0+2 hours, T0+3 hours, T0+4 hours: measurement of the sucrose consumption by weighing of the bottles. Followed by comparison of the experimental and control groups' intake of the sucrose solution.

In another test, the effect of a compound on the consumption of an alcohol solution can be assessed in mice. For instance, male C 57 BL 6 mice are isolated on the day of their arrival in an animal housing under a reverse cycle (night from 10 am to 10 pm) with 2 bottles filled with water. After 1 week, one of the bottles of water is replaced with a bottle filled with a 10% alcohol solution for 6 hours of the test. Each day, 30 minutes before the bottle of alcohol is introduced, the mice are treated with a compound for use according to the invention. The amounts of alcohol and water consumed are measured after 6 hours. The test is repeated for 4 days. The results for an experimental and a control or vehicle are compared. Cippitelli A, et al., also teach suitable methods for assessing the ability of an anandamide transport inhibitor to reduce ethanol self-administration. (see, Cippitelli, et al. Eur J Neurosci 26(2) pp. 476-86 (2007)).

Additional methods for screening a test compound for effects on an appetitive disorder, addiction or withdrawal are taught and withdrawals are known in the art. Scherma M et al. exemplify suitable methods of screening anandamide transport inhibitors on the rewarding effects of nicotine and nicotine-induced dopamine elevations in the nucleus accumbens shell in rats. Scherma M et al., Br J Pharmacol. (2011). Gomes et al. teach suitable measures for assessing the anxiolytic, obsession/compulsion-modulating effects of cannabinoids using the mouse marble-burying behavior (see Gomes et al., Behav Pharmacol. 21(4):353-8 (2010) and Gomes et al, Prog Neuropsychopharmacol Biol Psychiatry. 30; 35(2):434-8 (2011)). Del Arco I, et al., teach that amandamide transport inhibitors can be used to attenuate spontaneous opiate withdrawal in mice (see, Del Arco et al., Eur J Pharmacol 454(1) pp. 103-4 (2002)).

The effects of test compounds on feeding behavior can be analyzed in animals deprived of food for 24 hr and habituated to handling (see, Rodriguez de Fonseca F, et al., Nature 414:209-212 (2001)) or in partially satiated animals (i.e., 24 hr food-deprived animals allowed to eat for 60 min before drug testing) (Williams C M, et al., Physiol Behav 65:343-346. (1998)). 48 hr before testing, the bedding material is removed from the cage and a small can containing food pellets placed inside the cage for 4 hr. The animals are then food-deprived for 24 hr, with access to water ad libitum. The animals are placed in their home cage about 15 min after drug administration. A container with a known amount of food (usually 30-40 gm) and a bottle containing a known amount of fresh water are placed in the home cage. Food pellets and food spillage are weighed at one, two and four hours after starting the test, and the amount of food eaten recorded. The amount of water consumed is also measured. For partial satiation of animals, 24 hr food-deprived rats are allowed to eat from the container for 1 hr. Then, the container is replaced and food intake recorded. Fifteen minutes after drug injections, the food is again presented, and the amount consumed recorded hourly for 4 hr.

### —Methods for Screening for Antipsychotic or Antischizophrenic or Dopamine-Modulating Activity

Without being wed to theory, it is believed that excessive dopamine transmission in the CNS may contribute to schizophrenia and other mental disorders. Approximately one-third of all schizophrenic patients manifest obvious dopamine transmitter and/or receptor increases. Others who do not overtly manifest this abnormality still show improvement of symptoms with the pharmacological blockade of dopamine receptors. These dopamine receptor antagonists ultimately result in overall reductions in dopamine concentrations due to depolarization block and dopamine receptor antagonism. Thus, malfunction of neural circuits, many of which dopamine has a direct and/or indirect role in activating, appears to be involved in schizophrenic symptoms. As has been shown above, blocking dopamine receptors in subcortical areas of the brain substantially reduces schizophrenic symptoms. Generalized reduction of dopamine production in these areas provides similar relief to patients suffering from this disease. Cannabinoids have been found to modulate dopamine activity in the CNS.

Methods for screening compound for their effects on dopaminergic transmission and systems in the CNS are well known to one of ordinary skill in the art. Methods for conducting clinical trials of candidate agents in any of the above neurological diseases, disorders and conditions are well known to one of ordinary skill in the art. In addition Beltramo M, et al., teach methods for assessing the effects of anandamide transport inhibitors in reversing dopamine D(2) receptor responses (see, Beltramo et al., J Neurosci 20(9) pp. 3401-7 (2000).

Apomorphine-induced yawning can be measured in transparent plastic boxes (35×30×17 cm) following established procedures known in the art (see, Dourash et al., Neuropharmacology 28:1423-1425 (1989). Test compound or vehicle are administered 5 min before subcutaneous injection of apomorphine (80 ng/kg) or vehicle (aqueous 0.9% NaCl containing 40% DMSO, 0.2 ml/kg). Yawning is measured for a 30 min period after apomorphine injection.

Effects of anandamide inhibitors on basal and quinpirole-induced motor behaviors can be assayed as known in the art (see, Giuffrida A, et al., Nat Neurosci 2:358-363 (1999)). Animals are housed with a controlled photoperiod (lights on from 8:00 A.M. to 8:00 P.M.) and habituated to handling for 1 week before starting the experiments. Locomotor activity can be studied in an opaque open field (100×100×40 cm), the floor of which was marked with 20×20 cm squares. The field is illuminated using a ceiling halogen light that was regulated to yield 350 lux at the center of the field. Rats are habituated to the field for 10 min the day before testing. On the experimental day, the animals are placed in the center of the open field and locomotor activity (number of lines crossed) was scored during 5 min. Behavior is tested 5, 30, 60, and 120 min after the injection of either vehicle or drugs. Spontaneous motor behavior can be studied in a glass observation box (40×30×30 cm, one rat per box) and tested for 5 min at 5, 30, 60, and 120 min after drug injection. The tests were conducted in a sound-isolated room, illuminated with an indirect halogen light (125 lux). The behavior was videotaped on a video cassette recorder. Animals were placed in the box 5 min before the onset of the testing period. (1) Immobility (defined as complete absence of observable movement), (2) number of rearing episodes, (3) time spent grooming; (4) sniffing activity, and (5) total oral activity (yawning, vacuous chewing, and licking) are scored. Catalepsy is assessed by the bar test. At various times (0, 30, 60, or 120 min) after the injection of vehicle or drugs, the forepaws of test animals were positioned on a 10-cm-high bar, and the time spent by the animals in this position was measured. Catalepsy tests ended when the animals removed both forepaws from the bar with a cut-off time of 3 minutes. The observed were blinded to the experimental conditions.

The effects of anandaminde transport inhibitors can be studied using juvenile spontaneously hypertensive rats. The experimental system can be a Lk-maze, a 60×60×40 cm wooden box with a 30×30×40 cm plastic transparent smaller box inserted in the middle. Rats were allowed to explore the resulting corridor (60 cm long, 15 cm wide, and 40 cm high). A set of four such boxes are located in a sound-attenuated room. The experimental box is illuminated by a white, cold 4 W lamp placed 60 cm above the floor in the center of the wooden cover, providing 0.1-0.2 μW/cm². Six-week-old rats are exposed for 30 min to the Lk-maze after a single subcutaneous injection of the test compound or vehicle. Testing can be performed at the beginning of the light phase of the circadian cycle between 9:00 AM and 2:00 P.M., and the two members of each cage are tested simultaneously to minimize the interference with the arousal state. Behaviors were video recorded. Behavioral variables, i.e., the frequency of corner crossings as index of travel distance, duration of rearings on hindlimbs, and leanings against the walls with one or both forepaws, are assessed in 1 min blocks. At the end of the test, the number of fecal boli are counted, and the floor carefully cleaned with a wet sponge.

Methods of Use, Pharmaceutical Compositions, and their Administration

### —Methods of Use

### —Anxiety and Anxiety Related Disorders.

In some embodiments, the compounds of Formula I and II, and their pharmaceutical compositions and methods of administering them are useful in treating anxiety and anxiety disorders or conditions. The compounds and compositions are useful, for example in treating anxiety, clinical anxiety, panic disorder, agoraphobia, generalized anxiety disorder, specific phobia, social phobia, obsessive-compulsive disorder, acute stress disorder, and post-traumatic stress disorder; and adjustment disorders with anxious features, anxiety disorders due to general medical conditions, substance-induced anxiety disorders, and the residual category of anxiety disorder not otherwise specified. The treatment may be prophylactic or therapeutic. The treatment may be administered to a human subject. The compounds may be used in otherwise healthy individuals who are not otherwise in need of any pharmaceutical intervention for a disease or condition such as insomnia or for pain relief.

In some embodiments, the compounds methods, and compositions of the invention may also be administered to treat anxiety in mammals, including cats, dogs, and humans. In some embodiments, the compounds may be used in otherwise healthy individuals who are not in need of pharmaceutical interventions for any other disease or disorder than anxiety or an anxiety disorder.

The compounds and compositions of the invention may be administered solely for the purposes of reducing the severity or frequency of anxiety or an anxiety disorder. In some embodiments, the compounds are administered to treat post traumatic stress.

### Depression and Depressive Disorders

In some embodiments, the compounds of Formula I and their pharmaceutical compositions and methods of administering them are useful to elevate mood or treat depression and depressive disorders or conditions. The compounds and compositions are useful, for example in treating major depressive disorders (unipolar depression), dysthymic disorders (chronic, mild depression), and bipolar disorders (manic-depression). The depression may be clinical or subclinical depression. The treatment may be prophylactic or therapeutic. The treatment may be administered to a human subject. The compounds may be used in otherwise healthy individuals who are not otherwise in need of any pharmaceutical intervention for a disease such as insomnia or for pain relief

In some embodiments, the compounds methods, and compositions of the invention may also be administered to treat depression in mammals, including cats, dogs, and humans. In some embodiments, the compounds may be used in otherwise healthy individuals who are not in need of pharmaceutical interventions for any other disease or disorder than depression or a depressive disorder.

The compounds and compositions of the invention may be administered solely for the purposes of reducing the severity or frequency of depression or a depressive disorder.

### Use of FLAT Inhibitors to Control of Appetite and to Treat Appetite Disorders

In some embodiments, the invention provides pharmaceutical compositions and methods of using the compounds for use according to the invention to reduce appetite(s), reduce body fat and for treating or preventing obesity or overweight in a mammal and for preventing or treating the diseases associated with these health conditions. In one aspect of the instant invention, methods are provided for reducing appetite, body fat or body weight, or for treating or preventing obesity or overweight, or for reducing food intake, or treating an appetency disorder in a mammal by administering to the mammal the compound. In a further embodiment, the inhibitor is administered in a combination therapy with anandamide or another fatty acid alkanolamide compound, or a homologue or analog of oleylethanolamide or the fatty acid alkanolamide compound, which reduces appetite or food consumption and is subject to removal by FLAT.

In some embodiments, the compound for use according to the invention is administered to a subject in amounts sufficient to reduce body fat, body weight, or prevent body fat or body weight gain or to reduce appetite(s). In another aspect of the invention, pharmaceutical compositions are provided which comprise a first compound which is anandamide which reduces appetite or which has an effect to reduce appetite. In other aspects, the invention is drawn to such pharmaceutical compositions and their methods of use to reduce or control appetite or to treat appetite disorders.

In some embodiments, the compositions of the invention may be administered in therapeutically effect amounts to treat an appetency disorder in a subject. The treatment may be administered to a human subject. The disorder may be directed toward food, or a substance of abuse (e.g., alcohol, tobacco) of the subject.

### —Schizophrenia and Dopamine Related Disorders

Is some embodiments, the compounds for use according to the invention their pharmaceutical compositions and methods of administering them are useful in treating schizophrenia, psychosis and dopamine related disorders. The treatment may be prophylactic or therapeutic. The treatment may be administered to a human subject. The compounds may be used in otherwise healthy individuals who are not otherwise in need of any pharmaceutical intervention for a disease such as insomnia or hyperalgesia.

The compounds and compositions of the invention may be administered solely for the purposes of reducing the severity or frequency of a personality disorder, schizophrenia or dopamine related disorder. They may be administered to reduce paranoid ideation and flat affect.

### Use to Induce Sleep

In some embodiments, the compounds for use according to the invention may be administered to induce or promote sleep in a mammalian subject. The treatment may be prophylactic or therapeutic. The treatment may be administered to a human subject. The compounds and compositions of the invention may be administered solely for the purposes of reducing the severity or frequency or extent of sleeplessness.

### —Pain

In some embodiments, the compositions of the invention may be administered in therapeutically effect amounts to alleviate or treat pain in a subject in need thereof. The treatment may be prophylactic or therapeutic. The treatment may be administered to a human subject. The compounds and compositions of the invention may be administered solely for the purposes of reducing the severity or frequency or extent of pain. The treatment may be administered in a combination therapy with another pain reliever or anti-inflammatory agent. In some embodiments, the pain can be a neuropathic pain selected from the group consisting of post trigeminal neuralgia, neuropathic low back pain, peripheral or polyneuropathic pain, complex regional pain syndrome (causalgia and reflex sympathetic dystrophy), diabetic neuropathy, toxic neuropathy, and chronic neuropathy caused by chemotherapeutic agents. In other embodiments, the pain is renal and liver colic pain or fibromyalgia. In some neuropathic pain embodiments, the primary lesion or dysfunction of the nervous system is caused by a mechanical injury to a nerve of the subject. In a further embodiment, the mechanical injury is due to compression of a nerve, transection of nerve, causalgia, spinal cord injury, post surgical pain, phantom limb pain, or scar formation in the subject. Methods of diagnosing such pain is well known in the art.

In other embodiments, the pain is a pain caused by inflammation or injury of a tissue. Inflammatory pain develops in response to tissue damage occurring from the noxious stimuli. In response to the tissue injury, cytokines and other mediators are released which strengthen nociception. As a result primary hyperalgesia (increased sensitivity to pain) occurring in the area of injury and a secondary hyperalgesia occurring in the tissue surrounding the injury ensue. The hyperalgesia subsides with the inflammation as the tissue is healed. In some further embodiments, the inflammation is associated with pulmonary edema, kidney stones, minor injuries, wound healing, skin wound healing, vaginitis, candidiasis, lumbar spondylanhrosis, lumbar spondylarthrosis, vascular diseases, migraine headaches, sinus headaches, tension headaches, dental pain, periarteritis nodosa, thyroiditis, appen aplastic anemia, Hodgkin's disease, sclerodoma, rheumatic fever, type I diabetes, type II diabetes, myasthenia gravis, multiple sclerosis, sarcoidosis, nephrotic syndrome, Behcet's syndrome, polymyositis, gingivitis, hypersensitivity, swelling occurring after injury, or myocardial ischemia, or osteoarthritis.

### —Inflammation

In some embodiments, the compositions of the invention may be administered in therapeutically effect amounts to alleviate or treat inflammation in a subject. The treatment may be prophylactic or therapeutic. The treatment may be administered to a human subject. The compounds and compositions of the invention may be administered solely for the purposes of reducing the severity or frequency or extent of the inflammation. The treatment may be administered in a combination therapy with another pain reliever or anti-inflammatory agent. In some embodiments, the inflammatory disorder is asthma or arthritis.

### —Nausea

In certain embodiments, the treatment may be administered in therapeutically effect amounts to treat nausea in a subject in need thereof

### —Neurodegenerative Diseases

In certain embodiments, the treatment may be administered in therapeutically effect amounts to treat a neurodegenerative disease in a subject in need thereof. In some embodiments, the disease is Alzheimer's disease or multiple sclerosis

### Pharmaceutical Compositions.

The invention provides pharmaceutical compositions for modulation of anandamide activity in a subject. The term “composition”, as in pharmaceutical composition, is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and a pharmaceutically acceptable carrier. The term “pharmaceutical composition” indicates a composition suitable for pharmaceutical use in a subject, including an animal or human. A pharmaceutical composition generally comprises an effective amount of an active agent and a pharmaceutically acceptable carrier.

The pharmaceutical compositions which comprise compounds of the invention according to Formula I and a pharmaceutically acceptable carrier. The pharmaceutical compositions of the present invention comprise a compound of the instant invention as an active ingredient or a pharmaceutically acceptable salt thereof, and may also contain a pharmaceutically acceptable carrier and optionally other therapeutic ingredients.

The compositions include compositions suitable for oral, rectal, topical, parenteral (including subcutaneous, intramuscular, and intravenous), ocular (ophthalmic), pulmonary (nasal or buccal inhalation), or nasal administration, although the most suitable route in any given case will depend in part on the nature and severity of the conditions being treated and on the nature of the active ingredient. An exemplary route of administration is the oral route. The compositions may be conveniently presented in unit dosage form and prepared by any of the methods well-known in the art of pharmacy.

In practical use, the compounds of the invention can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, hard and soft capsules and tablets, with the solid oral preparations being preferred over the liquid preparations.

Because of their ease of administration, tablets and capsules represent the most advantageous oral dosage unit form in which case solid pharmaceutical carriers are obviously employed. If desired, tablets may be coated by standard aqueous or nonaqueous techniques. Such compositions and preparations can contain at least 0.1 percent of active compound. The percentage of active compound in these compositions may, of course, be varied and may conveniently be between about 2 percent to about 60 percent of the weight of the unit. The amount of active compound in such therapeutically useful compositions is such that a therapeutically effective dosage will be obtained. The active compounds can also be administered intranasally as, for example, liquid drops or spray.

The tablets, pills, capsules, and the like may also contain a binder such as gum tragacanth, acacia, corn starch or gelatin; excipients such as dicalcium phosphate; a disintegrating agent such as corn starch, potato starch, alginic acid; a lubricant such as magnesium stearate; and a sweetening agent such as sucrose, lactose or saccharin. When a dosage unit form is a capsule, it may contain, in addition to materials of the above type, a liquid carrier such as a fatty oil.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor. To prevent breakdown during transit through the upper portion of the GI tract, the composition may be an enteric coated formulation.

With respect to formulations with respect to any variety of routes of administration, methods and formulations for the administration of drugs are disclosed in Remington's Pharmaceutical Sciences, 17th Edition, (Gennaro et al. Eds., Mack Publishing Co., 1985). Remington's Pharmaceutical Sciences, Gennaro AR ed. 20th edition, 2000: Williams & Wilkins Pa., USA.

Preferably, all the ingredients are pharmaceutical grade materials. Preferably, they are sterile.

The compounds of the invention may also be administered parenterally. Solutions or suspensions of these active compounds can be prepared in water suitably mixed with a surfactant such as hydroxypropylcellulose. Dispersions can also be prepared in glycerol, liquid polyethylene glycols and mixtures thereof in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), suitable mixtures thereof, and vegetable oils.

The compounds of the invention can be effective over a wide dosage range. For example, in the treatment of adult humans, dosages from about 10 to about 1000 mg, about 100 to about 500 mg or about 1 to about 100 mg may be needed. Doses of the 0.05 to about 100 mg, and more preferably from about 0.1 to about 100 mg, per day may be used. A most preferable dosage is about 0.1 mg to about 70 mg per day. In choosing a regimen for patients, it may frequently be necessary to begin with a dosage of from about 2 to about 70 mg per day and when the condition is under control to reduce the dosage as low as from about 0.1 to about 10 mg per day. For example, in the treatment of adult humans, dosages from about 0.05 to about 100 mg, preferably from about 0.1 to about 100 mg, per day may be used. The exact dosage will depend upon the mode of administration, on the therapy desired, form in which administered, the subject to be treated and the body weight of the subject to be treated, and the preference and experience of the physician or veterinarian in charge.

Generally, the compounds of the present invention can be dispensed in unit dosage form comprising preferably from about 0.1 to about 100 mg of active ingredient together with a pharmaceutically acceptable carrier per unit dosage. Usually, dosage forms suitable for oral, nasal, pulmonary or transdermal administration comprise from about 0.001 mg to about 100 mg, preferably from about 0.01 mg to about 50 mg of the compounds admixed with a pharmaceutically acceptable carrier or diluent. For storage and use, these preparations preferably contain a preservative to prevent the growth of microorganisms.

Administration of an appropriate amount the candidate compound may be by any means known in the art such as, for example, oral or rectal, parenteral, intraperitoneal, intravenous, subcutaneous, subdermal, intranasal, or intramuscular. In some embodiments, administration is transdermal. An appropriate amount or dose of the candidate compound may be determined empirically as is known in the art. For instance, an appropriate or therapeutic amount is an amount sufficient to effect a loss of body fat or a loss in body weight in the animal over time and the candidate compound can be administered as often as required to effect a loss of body fat or loss in body weight, for example, hourly, every six, eight, twelve, or eighteen hours, daily, or weekly

Formulations suitable for oral administration can consist of (a) liquid solutions, such as an effective amount of the packaged nucleic acid suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

Injection solutions and suspensions can be prepared from sterile powders, granules, and tablets of the kind previously described. Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

With respect to transdermal routes of administration, methods for transdermal administration of drugs are disclosed in Remington's Pharmaceutical Sciences, Gennaro AR ed. 20th edition, 2000: Williams & Wilkins P A, USA. Dermal or skin patches are a preferred means for transdermal delivery of the compounds of the invention. Patches preferably provide an absorption enhancer such as DMSO to increase the absorption of the compounds. Other methods for transdermal drug delivery are disclosed in U.S. Pat. Nos. 5,962,012, 6,261,595, and 6,261,595. Each of which is incorporated by reference in its entirety.

Preferred patches include those that control the rate of drug delivery to the skin. Patches may provide a variety of dosing systems including a reservoir system or a monolithic system, respectively. The reservoir design may, for example, have four layers: the adhesive layer that directly contacts the skin, the control membrane, which controls the diffusion of drug molecules, the reservoir of drug molecules, and a water-resistant backing. Such a design delivers uniform amounts of the drug over a specified time period, the rate of delivery has to be less than the saturation limit of different types of skin.

The monolithic design, for example, typically has only three layers: the adhesive layer, a polymer matrix containing the compound, and a water-proof backing. This design brings a saturating amount of drug to the skin. Thereby, delivery is controlled by the skin. As the drug amount decreases in the patch to below the saturating level, the delivery rate falls.

Compounds of the invention may be used in combination with other compounds of the invention or with other drugs that may also be useful in the treatment, prevention, suppression of a neurological or psychological disorder. In one embodiment, the second drug is not a FLAT inhibitor and is directed toward the same disorder as the fatty acid amide inhibitor. Such other drugs may be administered, by a route and in an amount commonly used therefor, contemporaneously or sequentially with a compound of the invention. When a compound of the invention is used contemporaneously with one or more other drugs, a pharmaceutical composition in unit dosage form containing such other drugs and the compound is preferred.

When used in combination with one or more other active ingredients, the compound of the present invention and the other active ingredients may be used in lower doses than when each is used singly. Accordingly, the pharmaceutical compositions of the present invention include those that contain one or more other active ingredients, in addition to the compounds disclosed above. For example, a FLAT inhibitor according to Formula I may be formulated with an anxiolytic agent which is not a FLAT inhibitor. For example, a FLAT inhibitor according to Formula I may be formulated with an antidepressant.

In the pharmaceutical compositions of the present invention for oral, sublingual, subcutaneous, intramuscular, intravenous, transdermal, local or rectal administration, the active principle, by itself or in association with another active principle, can be administered to animals and humans in unit dosage forms of administration mixed with conventional pharmaceutical carriers. The appropriate unit forms of administration include oral forms such as tablets, gelatin capsules, powders, granules and solutions or suspensions to be taken orally, sublingual and buccal forms of administration, aerosols, implants, subcutaneous, intramuscular, intravenous, intranasal or intraocular forms of administration and rectal forms of administration.

In other embodiments, the pharmaceutical compositions of the present invention, the active principle or active principles are generally formulated in dosage units. The dosage unit contains from 0.5 to 1000 mg, advantageously from 1 to 500 mg and preferably from 2 to 200 mg of FLAT inhibitor per dosage unit for daily administration.

### General Methods for Preparing Compounds of Formula I

The compounds of Formula I can be prepared through a process consisting of standard synthetic transformations reported, for instance, in Michael Smith, Jerry March—March's Advanced Organic Chemistry: reactions mechanisms and structure—6th Edition, John Wiley & Sons Inc., 2007, which is incorporated herein as reference. It is well known to one of ordinary skill in the art that transformation of a chemical function into another may require that one or more reactive centers in the compound containing this function be protected in order to avoid undesired side reactions. Protection of such reactive centers, and subsequent de-protection at the end of the synthetic transformations, can be accomplished following standard procedures described in Theodora W. Green and Peter G. M. Wuts—Protective Groups in Organic Synthesis, Fourth Edition, John Wiley & Sons Inc., 2006, which is incorporated herein as reference.

Preferred compounds of Formula I, where m is 0, X₁ and X₂ are N, X₃ and X₄ are C, Y is NH, Z₁ is C═O, Z₂ is NH, and p is 0, are analogs of compound ARN272 that can be represented by Formula II.

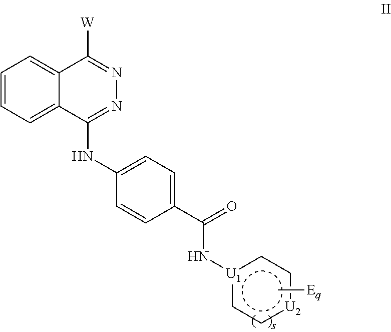

Compounds of Formula II, wherein W represents an aryl or heteroaryl group, U₁, U₂, E, s, and q are as described above, can be prepared applying synthetic procedures described for instance in, but not limited to, Evgueni L. Piatniski et al., Bioorganic and Medicinal Chemistry 15, 4696-4698 (2005) or in Matthew A. J. Duncton et al., Bioorganic and Medicinal Chemistry 17, 731-740 (2009) or in Victor J. Cee et al., Journal of Medicinal Chemistry 53, 6368-6377 (2010), as depicted in Scheme 1.

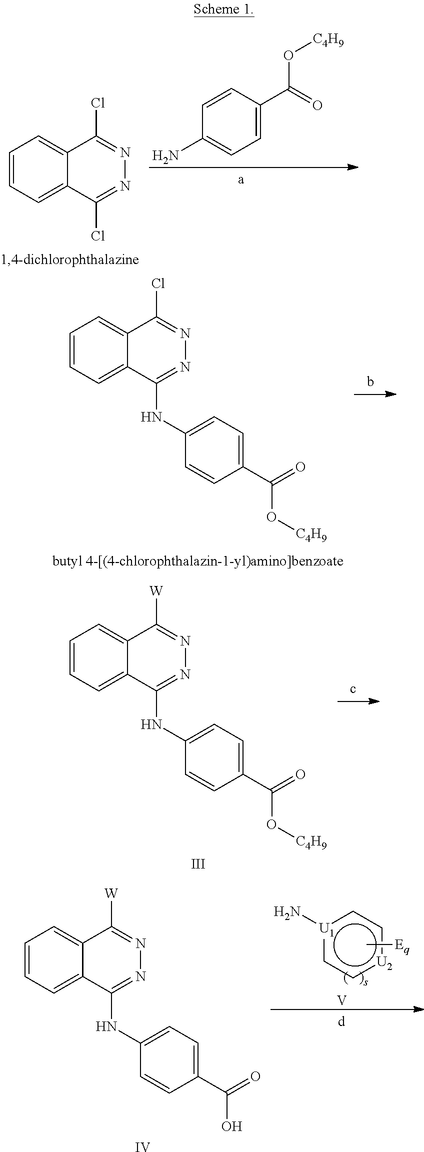

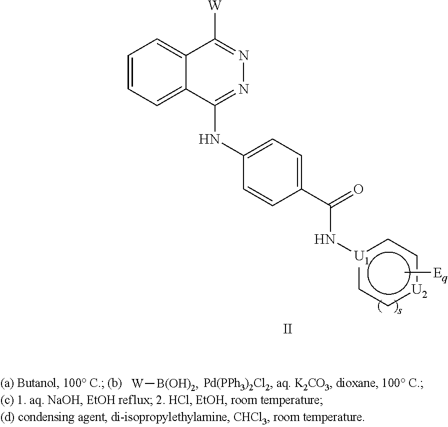

According to the procedure reported in Scheme 1, the commercially available 1,4-dichlorophthalazine is reacted with butyl-4-aminobenzoate in butanol at 100° C. to obtain butyl 4[(4-chlorophthalazin-1-yl)amino]benzoate. The latter compound can be reacted with a boronic acid of formula W—B(OH)₂, wherein W represents an aryl or heteroaryl group, in the presence of a suitable catalyst, such as bis-(triphenylphosphine) palladium (II) dichloride, in a suitable solvent, such as dioxane, in the presence of a suitable base, such as aqueous potassium carbonate, and at a suitable temperature, such as 100° C., to obtain a compound represented by Formula III. A compound of Formula III can be further elaborated by hydrolysis of the butyl ester to the acid of Formula IV, wherein W represents an aryl or heteroaryl group, which is subsequently reacted with an amine of Formula V, wherein U₁, U₂, E, s, and q are as defined above, to obtain a compound of Formula II. The hydrolysis of the butyl ester can be accomplished by reaction with an aqueous inorganic base, such as sodium hydroxide, in a suitable solvent, such as ethanol, and at a suitable temperature, such as refluxing of the solvent. Treatment of the resulting carboxylate salt with a suitable acid, such as hydrochloric acid, in a suitable solvent, such as ethanol, and at a suitable temperature, such as room temperature, yields an acid of Formula IV. An acid of Formula IV is transformed into a compound of Formula II by reaction with an amine of Formula V, wherein U₁, U₂, E, s, and q are as defined above, in the presence of a suitable condensing agent, such as 0-(7-azabenzotriazol-1-yl)-N,N,N′,N′-tetramethyluronium hexafluorophosphate or O-benzotriazole-N,N,N′,N′-tetramethyl-uronium-hexafluorophosphate or 1-hydroxybenzotrizole or N,N-di-isopropylcarbodiimide, in a suitable solvent, such as chloroform, in the presence of an organic base, such as di-isopropylethylamine or triethylamine, and at a suitable temperature, such as room temperature.

Preferred compounds of Formula II are those where W represents an aryl or heteroaryl group, U₁ and U₂ are C, s and q are each 1, and E represents an alkoxy or OH. These compounds are close analogs of the compound ARN272, and can be prepared from a compound of Formula IV applying the general procedure reported in Scheme 1, as detailed in Scheme 2.

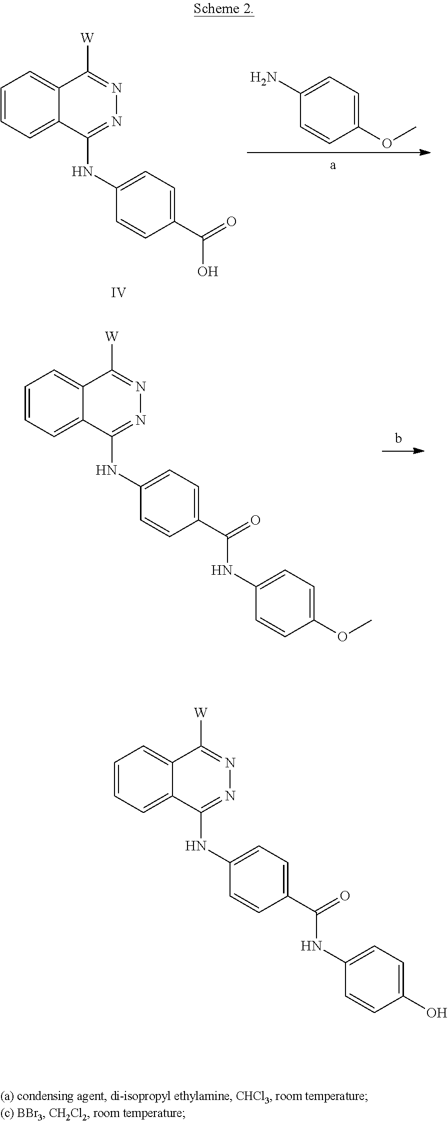

A compound of Formula IV can be reacted with 4-methoxyaniline in the presence of a suitable condensing agent, such as O-(7-azabenzotriazol-1-yl)-N,N,N′,N′-tetramethyluronium hexafluorophosphate, in a suitable solvent, such as chloroform, in the presence of an organic base, such as di-isopropylethylamine, and at a suitable temperature, such as room temperature, thus obtaining a compound of Formula II, wherein W represents an aryl or heteroaryl group, U₁ and U₂ are C, s and q are each 1, and E represents a methoxy group. A compound of Formula II, wherein W represents an aryl or heteroaryl group, U₁ and U₂ are C, s and q are each 1, and E represents a methoxy group, can be transformed into another compound of Formula II, wherein W represents an aryl or heteroaryl group, U₁ and U₂ are C, s and q are each 1, and E represents an OH group, by treatment with a suitable demethylating agent, such as boron tribromide, in a suitable solvent, such as dichloromethane, and at a suitable temperature, such as room temperature.

### Preparation of compound ARN272, 4-{[4-(4-hydroxyphenyl)phthalazin-1-yl]amino-N-phenylbenzamide}

The compound ARN272 can be prepared applying the synthetic procedures reported in Scheme 1 and in Scheme 2, as detailed in Scheme 3.

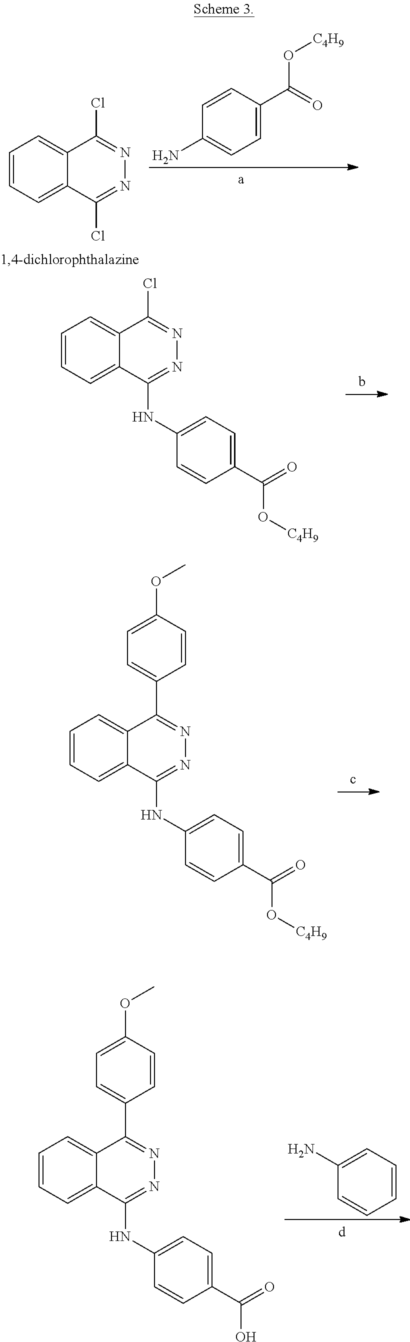

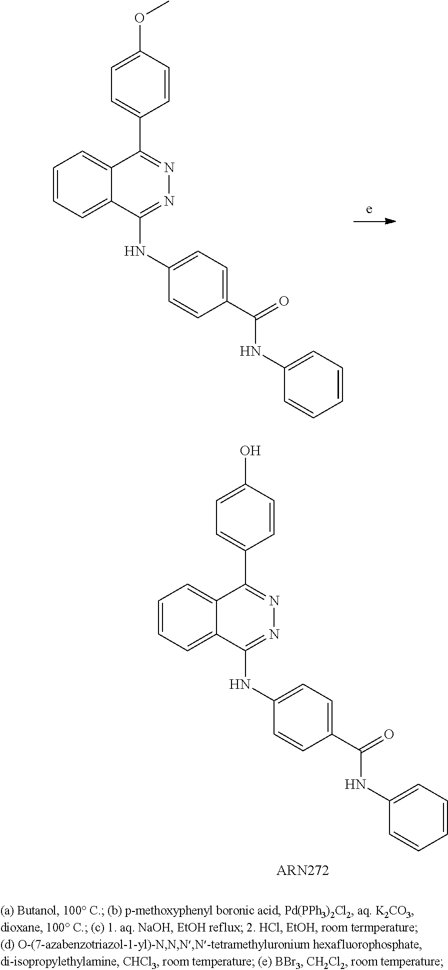 [SMILES: COc1ccc(-c2nnc(Nc3ccc(C(=O)Nc4ccccc4)cc3)c3ccccc23)cc1 ; O=C(Nc1ccccc1)c1ccc(Nc2nnc(-c3ccc(O)cc3)c3ccccc23)cc1]

### Preparation of compound N-(4-hydroxyphenyl)-4-[[4-(4-hydroxyphenyl)phthalazin-1-yl]amino]benzamide

The compound N-(4-hydroxyphenyl)-4-[[4-(4-hydroxyphenyl)phthalazin-1-yl]amino]benzamide, a close analog of compound ARN272, can be prepared applying the synthetic procedures reported in Scheme 1 and in Scheme 2, as detailed in Scheme 4.

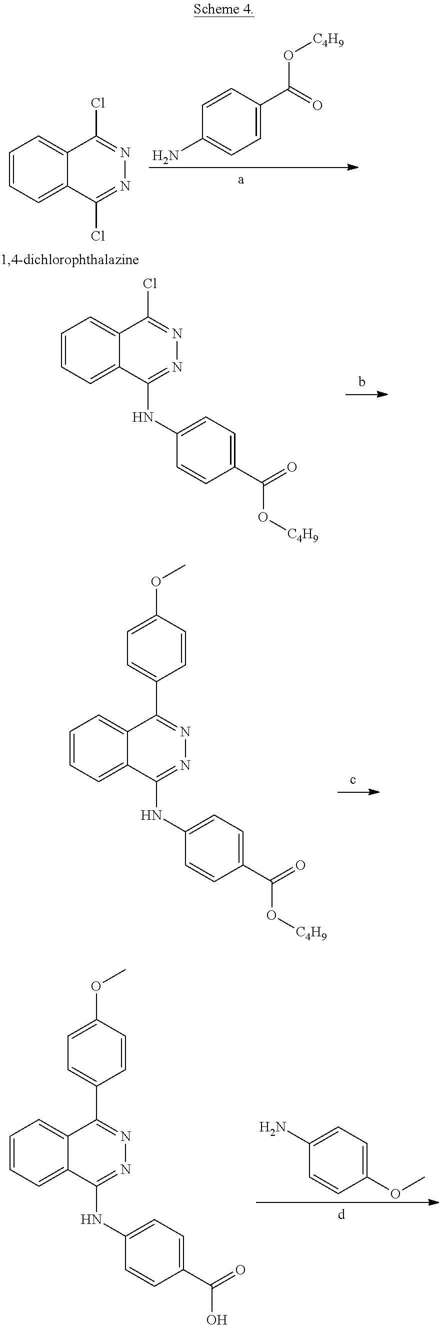

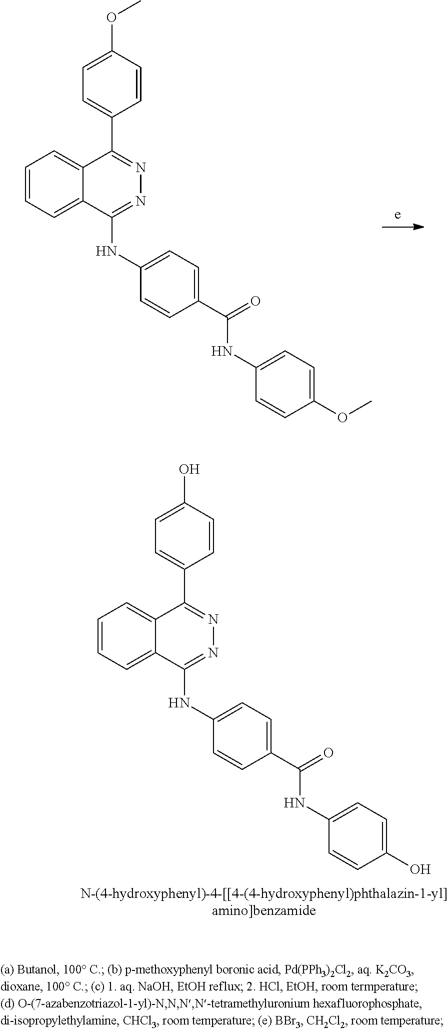 [SMILES: COc1ccc(NC(=O)c2ccc(Nc3nnc(-c4ccc(OC)cc4)c4ccccc34)cc2)cc1 ; O=C(Nc1ccc(O)cc1)c1ccc(Nc2nnc(-c3ccc(O)cc3)c3ccccc23)cc1]

The following examples are provided for illustrative purposes, and are not intended to limit the scope of the invention as claimed herein. Any variations in the exemplified articles and/or methods which occur to the skilled artisan are intended to fall within the scope of the invention. It will be obvious to one of skill in the art that changes and modifications may be practiced within the scope of the appended claims. The scope of the invention should, therefore, be determined not with reference to the above description, but should instead be determined with reference to the following appended claims, along with the full scope of equivalents to which such claims are entitled.

### EXAMPLES

### Materials and Methods

FLAT Model Preparation

A computational model of FLAT was created by removing the α2 helix (residues 37 to 68) from the crystal structure of FAAH-1-ΔTM (PDBid: 1MT5) (Bracey, M. H. et al., Science, 298, 1793 (2002)) and modeling positions 69 to 75 de novo, according to the primary sequence of FLAT. The co-crystallized ligand MAFP and water molecules were also removed. Hydrogen atoms and missing heavy atoms were added. Missing side chains of residues 69-75, zero occupancy side chains, and polar hydrogen atoms were optimized and assigned the lowest energy conformations. Tautomeric states of histidines and the positions of asparagine and glutamine side chain amidic groups were optimized to improve the hydrogen-bond pattern.

### Monte Carlo Simulations

The molecular system was described using internal coordinate variables. Protein atom types and parameters were taken from a modified version of the ECEPP/3 force field (Nemethy, G. et al., J. Phys. Chem., 96, 6472 (1992)). Conformational analysis of the α2-interacting loop was performed using the Metropolis Biased Probability Monte Carlo (BPMC) method as implemented in ICM (Abagyan, R. et al., J Mol Biol, 235, 983 (1994); Abagyan, R. A. et al., Journal of Computational Physics, 151, 402 (1999)). The initial temperature was set at 600 K. A total of 5×10⁷ energy evaluations were carried out. The variables sampled during the simulation were the backbone and side chain torsional angles of the loop amino acids; the variable minimized before acceptance/rejection according to the Metropolis criterion were the backbone and side chain torsional angles of the loop amino acids and the side chain torsional angles of all the amino acids with at least one heavy atom within 2 Å from the loop.

### Molecular Dynamics Simulations

Molecular dynamics simulations were conducted using the NAMD 2.6 (Phillips, J. C. et al., J Comput Chem, 26, 1781 (2005)) software and the Amber99SB force field (Hornak, V. et al., Proteins, 65, 712 (2006)). FLAT and FAAH-1-ΔTM were solvated in explicit solvent. The dimensions of the simulation box were chosen so to keep a margin of at least 8 Å from the solute in each coordinate direction, and the electro-neutrality of the cell was reached by adding counter-ions. The simulations were performed in the isobaric-isothermal statistical ensemble with periodic boundary conditions. Langevin dynamics was undertaken at the nominal temperature of 300 K with a frictional coefficient of 5 ps⁻¹ at the target pressure of 1 atm. A uniform time-stepping of 2 fs was used, while bonds involving hydrogen atoms were restrained to their reference value. Short-range non-bonded interactions were calculated using a distance cutoff of 10 Å together with a switching function acting at distances larger than 8 Å. Long-range electrostatics was taken into account using the Particle Mesh Ewald method (Essmann, U. et al., The Journal of Chemical Physics, 103, 8577 (1995)). The system was thermalized during 150 ps of molecular dynamics while smoothly releasing restraints initially applied onto the protein heavy atoms. The system was then allowed to evolve for a total simulated time of 100 ns. Analysis of the trajectory was performed using the VMD-1.8.6 (Humphrey, W. et al., Journal of Molecular Graphics, 14, 33 (1996)) and PLUMED 1.0 (Bonomi, M. et al., Computer Physics Communications, 180, 1961 (2009)) software.

### Electrostatic Simulations

To describe the influence of the α2 helix on the electrostatic environment of catalytic Lys¹⁴² (FAAH-1 numbering), the Poisson-Boltzmann equation was solved over a system in which the protein was modeled as a polarizable solute and the solvent was modeled as a high-dielectric continuum plus an ionic strength of 0.145M (Honig, B. et al., Science, 268, 1144 (1995)). The electrostatic potential values generated by representative structures of FLAT and FAAH-1-ΔTM at the zeta nitrogen site of Lys¹⁴² were calculated and compared using the DelPhi software (Rocchia, W. et al., The Journal of Physical Chemistry, B 105, 6754 (2001)). The simulations suggested that the α2 helix brings a positive electrostatic contribution to the lysine site. This contribution is the sum of the mere helix dipolar potential and the overall net charge potential generated by titratable residues borne by the α2 helix. In FLAT, this effect is only partially compensated by the reaction field generated by the solvent that replaces the α2 helix. The pKa shift between the two forms was estimated quantitatively by performing four electrostatic calculations according to the following conceptual thermodynamic cycle:

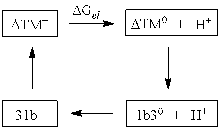

Here, the superscripts “+”/“0” refer to the charged/neutral state of lysine 142 in FAAH-1-ΔTM, and the corresponding lysine 74 in FLAT. The pKa was estimated according to the following formula:

ΔpKa=pKa(FLAT)−pKa(FAAHΔTM)=[(G_{1b}⁰−G_{1b}⁺)−(G_{1b}⁰−G_{1b}⁺)]/(2.303RT).

This calculation provided a positive average pKa shift of about 0.8 pKa units, pointing to an increased probability of having a protonated catalytic lysine in the FLAT form.

### Virtual Ligand Screening

Database Selection and Pre-Filtering.

The compounds for virtual ligand screening (VLS) were selected from the Molcart database v.1.9.6 (Molsoft, La Jolla, Calif.), which comprises 4,325,889 unique publicly available structures collected from vendors' catalogues. Two pre-filtering steps were performed on the database before carrying out structure-based screening via docking simulations. First, compounds were filtered using the physicochemical profile expected of a potential transport inhibitor; the parameters adopted are listed in the following Table.

| Parameter | Range |
|---|---|
| Molecular Weight (Da) | 300-400 |
| LogP | 4-8 |
| H-bond Donors | 0-3 |
| H-bond Acceptor | 2-4 |
| Polar Surface Area (Å²) | 20-80 |
| Molecular Volume (Å³) | 340-485 |
| Ring Terms | 3-7 |

This profile was defined using 5 template molecules: i) anandamide; ii) AM404: iii) a competitive and reversible FAAH thiohydantoin inhibitor (compound 123 in Seierstad, M. et al., J Med Chem, 51, 7327 (2008)); iv) OL-92, a very potent yet reversible FAAH inhibitor (Boger, D. L. et al., J Med Chem, 48, 1849 (2005)); and v) the leaving group of URB597 (Mor, M. et al., J Med Chem, 47, 4998 (2004)), which facilitates the formation of the Michaelis complex and occupies the solvent exposed region of the binding pocket (Mileni, M. et al., J Mol Biol, 400, 743 (2010)). The first filtering step retained 288,307 compounds, which matched the physicochemical profile of a potential transport inhibitor. To avoid a bias toward known scaffolds, molecules within a Tanimoto distance between 0 and 0.6 (with 0 indicating that two molecules share an identical fingerprint generated according to the Daylight algorithm—Daylight Chemical Information Systems Inc., Laguna Niguel—Calif.) from any of the compounds i)-v) were deleted from the database. This allowed us to improve the chemical diversity and potentially identify novel scaffolds. At the end of the pre-filtering steps, 122,095 unique molecules were selected for subsequent structure-based VLS simulations.

Docking Procedure.

The volume of the binding pocket in FLAT was calculated using the Pocketome Gaussian Convolution algorithm (An, J. et al., Mol Cell Proteomics, 4, 752 (2005)). The FLAT binding site was defined selecting all the residues with at least one non-hydrogen atom within 3.5 Å of the calculated volume, and extended towards both the acyl chain binding pocket and the cytosolic channel. The binding pocket was described by five 0.5 Å spacing potential grid maps, representing van der Waals potentials for hydrogens and heavy atoms, electrostatics, hydrophobicity, and hydrogen bonding. A soft form of the van der Waals potentials truncated at 2 kcal/mol was adopted. The three-dimensional models of 122,095 molecules, obtained from the previous pre-filtering steps, were built by automatically assigning bonds order, tautomeric form, stereochemistry, hydrogen atoms, and protonation state. Atom types and charges were taken from the MMFF force field (Halgren, T. A., Journal of Computational Chemistry, 17, 490 (1996)). Ligands were iteratively docked at the binding site. Docking was performed as a global energy optimization by means of the Biased Probability Monte Carlo stochastic procedure (Abagyan, R. et al., J Mol Biol, 235, 983 (1994)) as implemented in ICM3.7 (Molsoft). The binding energy was assessed by the ICM empirical standard scoring function and all compounds were ranked accordingly (Totrov, M., Abagyan, R., in Drug-receptor thermodynamics: introduction and applications R. B. Raffa, Ed. (Wiley, Chichester; New York, 2001) pp. 603-624). This step identified 4,601 compounds that matched the energy criterion of −32 kcal/mol, which is considered a threshold value for good binders (Totrov, M., Abagyan, R., paper presented at the RECOMB '99. Proceedings of the Third Annual International Conference on Computational Molecular Biology, Lyon (France) (1999)).

Cluster Analysis and Visual Inspection.

The compounds obtained from the docking-based VLS step were submitted to a chemical clustering procedure carried out using an average linkage hierarchical agglomerative approach. The dissimilarity matrix was calculated assessing the Tanimoto distance between the Daylight fingerprints of the compounds. The functional partition was selected at a threshold value of 0.5. This returned 56 unique clusters. Depending on the cluster cardinality, one to three representative compounds were selected. This provided 124 molecules as potential FLAT binders. After visual inspection 53 compounds were selected and 46, which were available from commercial vendors, were subjected to testing in vitro.

### Chemicals

Anandamide, AM404, VDM-11, UCM-707 and AMG9810 were purchased from Tocris Inc. (Ellisville, Mo.). 2-Carrageenan, AM251, AM630, PEG 400, and Tween-80 were from Sigma-Aldrich (Milan, Italy). ARN272 [(4-(5-(4-hydroxy-phenyl)-3,4-diaza-bicyclo[4.4.0]deca-[(6),2,4,7,9-pentaen-2-ylamino)-phenyl)-phenylamino-methanone] was from Ambinter (Paris, France). The commercial product was subjected to purification by liquid chromatography and its identity was confirmed by mass spectrometry and 1H-nuclear magnetic resonance spectroscopy. Its final purity was >95%. [3H]-Anandamide, [3H]-OEA, [3H]-PEA and [3H]-arachidonic acid were from American Radiolabeled Chemicals (Saint Louis, Mo.) and [3H]-CP-55940 was from Perkin Elmer (Boston, Mass.). [2H4]-Anandamide, [2H4]-OEA and [2H4]-PEA were from Cayman Chemicals (Ann Arbor, Mich.). URB597 was synthesized as described (Mor, M. et al., J Med Chem, 47, 4998 (2004)).

### Animals

Male CD1 mice, weighing 25-30 g (Charles River, Calco, Italy or Wilmington, Mass., USA) were used. They were housed in ventilated cages with free access to food and water. All cages had autoclaved cellulose paper as nesting material. Animals were maintained under a 12 h light/dark cycle at controlled temperature and relative humidity.

### Neurons in Primary Cultures

Primary cultures of rat cortical neurons were prepared as described (Evans, M. S. et al., J Neurosci Methods, 79, 37 (1998)). Briefly, cerebral cortices from 18 day-old rat embryos were dissected and cells were dissociated gently with a glass Pasteur pipette in phosphate-buffered saline (PBS, 10 mM, pH=7.5) containing glucose (6%, weight/volume). The cells were plated in 6-well plates (at a density of 5×10⁵ cells per well) coated with poly-(L)-ornithine (10 μg/ml) and poly(DL)-lysine (100 μg/ml), and cultured in Eagle's Minimum Essential Medium (MEM) supplemented with heat-inactivated horse serum (5%, vol/vol) and fetal bovine serum (FBS, 5%, vol/vol), glucose (30 mM), L-glutamine (2 mM), penicillin (10 units/ml), streptomycin (10 μg/ml) and HEPES (15 mM, pH 7.4). After 3 days in culture, media were replaced with Neurobasal (Invitrogen, Carlsbad, Calif.) supplemented with B-27 containing L-glutamine (0.1 mM), streptomycin (10 μg/ml) and penicillin (10 units/ml). The cultures were maintained at 37° C. in a humidified atmosphere of 95% air and 5% CO₂.

### Cloning and Expression of FLAT

Rat FLAT mRNA was amplified by polymerase chain reaction (PCR) using the following primers: forward, ACCATGGTGCTGAGCGAAGTGTGGACC (SEQ ID NO:4); reverse, TCACGATGGCTGCTTTTGAGGGGT (SEQ ID NO:5). An rFLAT amplicon was subcloned into the pCR®II vector using the TOPO® TA Cloning® kit (Invitrogen) and splicing was confirmed by DNA sequencing. Amplicons of rat FAAH-1, FLAT and mutated FLAT-S170G were cloned into a pcDNA3.1 expression vector (Invitrogen) under the control of human cytomegalovirus promoter. The plasmids contained a neomycin-resistance gene to provide stable selection with G418. HEK293 cells were cultured in Dulbecco's-modified Eagle's medium (DMEM) supplemented with FBS (10% vol/vol) and transfected with Lipofectamine™ 2000 (10 μl, Invitrogen) containing 1 μg of plasmids. 18 h after transfection, the culture media were replaced with supplemented DMEM containing G418 (0.2 mg/ml, Calbiochem, San Diego, Calif.). After 4 weeks in culture, surviving clones were isolated and analyzed by Western blot to select cell lines stably expressing the transgenes. Serine 173 residue in rat FLAT was mutated to glycine using the GeneTailor™ mutagenesis system (Invitrogen), following manufacturer's instructions.

### Ribonuclease Protection Assays

Probes comprising nucleotides 20-309 of FAAH-1 and nucleotides 20-220 of FLAT were generated by PCR followed by subcloning into pCR®II vectors (Invitrogen). The constructs were linearized by digestion with BamH I and used as template for in vitro transcription incorporated with ³²P-dCTP (MP Biomedicals, Solon, Ohio) by using RNA polymerase SP6 (Roche). Ribonuclease protection assays were performed using an RPA III kit (Applied Biosystems, Austin, Tex.) following manufacturer's instructions. Briefly, total RNA (30 μg) was applied for hybridization. Ribonuclease T1 and ribonuclease A were used for the digestion of unhybridized RNA and cRNA probe. The protected fragments were resolved on 5% acrylamide gel.

### Southern Blot Analyses

Total RNA was extracted from tissues with TRIzol™ (Invitrogen) and quantified spectrophotometrically. cDNA was synthesized from 20 μg of total RNA using SuperscriptII RNase H-reverse transcriptase (Invitrogen) following manufacturer's instructions. cDNA was digested with BlpI and electrophoresed at 30V overnight. Southern blots were performed using a standard protocol (Pearson, G. D. et al., Proc Natl Acad Sci USA, 79, 2976 (1982)) with a 5′-terminal probe (237 bp, from 41-277 bp) or a 3′-terminal probe (409 bp, from 601-1010 bp) for FAAH-1.

### FLAT Purification and Binding Assays

A pGEX-rFLAT plasmid containing fused rat FLAT and GST genes was constructed into a pGEX-4T vector (Amersham Biosciences, Piscataway, N.J.) and digested with BamH 1. The GST-FLAT fusion protein was generated in E. coli (BL21 strain, Novagen, San Diego, Calif.). E. coli were cultured in 2× yeast extract medium and induced with isopropyl-1-thio-β-D-galactopyranoside (IPTG) (0.1 mM). After induction, bacteria were grown at 27° C. for 10 h and harvested by centrifugation. The GST-FLAT fusion protein was purified using glutathione-sepharose beads (Amersham Biosciences), following manufacturer's instructions. Purified GST-FLAT was incubated at 25° C. for 2 h in HEPES buffer (50 mM, pH 7.0) containing KCl (50 mM), EDTA (5 mM), dithiothreitol (10 mM), and [³H]-anandamide (15 Ci/mmol). Free from bound [³H]-anandamide was separated on a Sephadex G-25 spin column (Amersham Biosciences) and radioactivity in the bound fraction was measured by liquid scintillation counting. Non-specific binding was determined in the presence of non-radioactive anandamide (10 μM).

### Anandamide Translocation Assays

6-well plates containing vector-transfected Hek293 cells, FLAT-expressing Hek293 cells, or rat cortical neurons in primary cultures (5×10⁵ per well) were incubated for 5 min at 37° C. in Tris-Krebs buffer (pH 7.5, 0.5 ml) containing [³H]-anandamide, [³H]-OEA, [³H]-PEA or [³H]-arachidonic acid (each at 200 nM; 10,000 dpm, specific activity 20 Ci/mmol). The cells were rinsed twice with Tris-Krebs buffer, scraped twice into sodium hydroxide (0.2 N, 0.5 ml) and transferred into scintillation vials. For cis-inhibition assays, test compounds were added to the cultures 10 min before addition of [³H]-anandamide. All experiments were conducted in triplicate.

### FAAH Assays

Cells were harvested and subjected to sonication in Tris-HCl buffer (50 mM, pH 8.0) containing sucrose (0.32 mM). Debris was removed by centrifugation for 5 min at 1,000×g, and protein concentration measured using a commercial kit (Bio-Rad, Hercules, Calif.). FAAH activity was determined at 37° C. for 30 min in Tris-HCl buffer (50 mM, pH 8.5) containing fatty acid-free bovine serum albumin (0.05%, weight/vol), protein (50 ng) and [³H]-anandamide (10,000 dpm, specific activity 20 Ci/mmol). After stopping the reactions with chloroform/methanol (1:1, vol/vol, 1 ml), radioactivity was measured in the aqueous layers.

### Western Blot Analyses

Cell extracts (20 ng protein) were subjected to electrophoresis on 4-15% SDS-PAGE gels and transferred onto Immobilon™ membranes (Millipore, Billerica, Mass.). Western blots were run using antibodies against V5 (1:3,000, Invitrogen), FAAH-1 (1:500, Abbiotec, San Diego, Calif.) and β-actin (1:10,000, Calbiochem). Bands were visualized with an Electrochemiluminescence Plus kit (Amersham Biosciences). Quantitative analyses were performed using the National Institutes of Health Image software, using β-actin as an internal standard.

### Cannabinoid Receptor Binding Assays

Brain membrane protein (50 μg) was incubated at 30° C. for 1 hr in a buffer (Tris-HCl, 50 mM; 5 mM MgCl₂; 2.5 mM EDTA; pH 7.5) containing [³H]-CP55940 (1 nM, 165 Ci per mmol). Non-specific binding was determined in the presence methyl arachidonyl fluorophosphonate (10 μM). Reactions were stopped by filtration through 0.5% polyethyleneimine-pretreated GF/B glass fibre filters (Whatman, Piscataway, N.J.). Bound radioligand was separated from free radioligand by washing the filter plate twice with Tris-HCl (50 mM, pH 7.4) and radioactivity was measured by liquid scintillation. Assays were performed in quadruplicate.

### Fatty Acid Amide Measurements Ex Vivo

CD1 mice received intraperitoneal injections of vehicle (5% polyethylene glycol 400-5% Tween-80 in saline, 5 ml/kg) or ARN272 (1 mg/kg) and were sacrificed 1 h or 2 h later under isoflurane anaesthesia. Blood was collected through a left cardiac puncture and centrifuged at 2000×g for 30 min. Plasma (0.2 ml) was incubated with 1 ml cold acetone, centrifuged at 1,500×g for 15 min at 4° C. and suspended in 50% methanol (2 ml) containing the following deuterium-containing standards: [²H₄]-anandamide, [²H₄]-OEA, [²H₄]-PEA and [²H₄]-2-AG. Lipids were extracted with chloroform (2 ml) and organic phases were collected and dried under nitrogen. Lipids were reconstituted in methanol (0.1 ml) and measured by isotope-dilution liquid chromatography/mass spectrometry (Fu, J. et al., J Biol Chem, 282, 1518 (2007)).

### Behavioral Tests

Behavioral experiments were performed in accordance with the Ethical Guidelines of the International Association for the Study of Pain and approved by Italian regulations on protection of animals used for experimental and other scientific purposes (D.M. 116192) as well as with European Economic Community regulations (O.J. of E.C. L 358/1 Dec. 18, 1986). Behavioral testing was performed between 9:00 a.m. and 5:00 p.m., in a quiet room, during the light cycle. Mice were used once and were sacrificed at the end of the experiment by cervical dislocation. edema was elicited by injecting λ-carrageenan (1% weight/vol in sterile water, 20 μl) into the left hindpaw of mice. Edema was measured with a plethysmometer (Ugo Basile, Comerio, Italy) at different time intervals. Drug solutions were prepared immediately before use in saline containing 10% PEG-400 and 10% Tween 80. Mechanical hyperalgesia was determined by measuring the latency in seconds to withdraw the paw away from a constant mechanical pressure exerted onto the dorsal surface. A 15-g calibrated glass cylindrical rod (diameter=10 mm) chambered to a conical point (diameter=3 mm) was used to exert the mechanical force. The weight was suspended vertically between two rings attached to a stand and was free to move vertically. A cutoff time of 180 s was used. Withdrawal thresholds were measured on both the inflamed paw (ipsilateral) and not inflamed paw (contralateral) 2, 4, 6 and 24 hours after carrageenan injection. Thermal hyperalgesia was assessed by the method of Hargreaves et al. (1988) by measuring the latency to withdraw the hind paw from a focused beam of radiant heat (thermal intensity: infrared 3.0) applied to the plantar surface using a plantar test apparatus (Ugo Basile). The cutoff time was set at 30 s. Withdrawal latency were measured on both inflamed and not-inflamed paws ( ) 2, 4, 6 and 24 hours after carrageenan injection. Vehicle or ARN272 (0.01-3 mg/kg, intraperitoneal) were injected immediately before carrageenan. AM251, AM630 and AMG 9810 (1 mg/kg, intraperitoneal) were injected 30 min before carrageenan.

### Statistical Analyses

Results are expressed as the mean±s.e.m of n separate experiments. The significance of differences between groups was evaluated by one-way analysis of variance (ANOVA) followed by a Dunnett's test for multiple comparisons, or a Student's t-test. For behavioral experiments, differences were evaluated by two-way ANOVA following by a Bonferroni's test. Analyses were conducted using the GraphPad Prism software (GraphPad Software, San Diego, Calif.), and differences were considered significant if P<0.05.

### Results

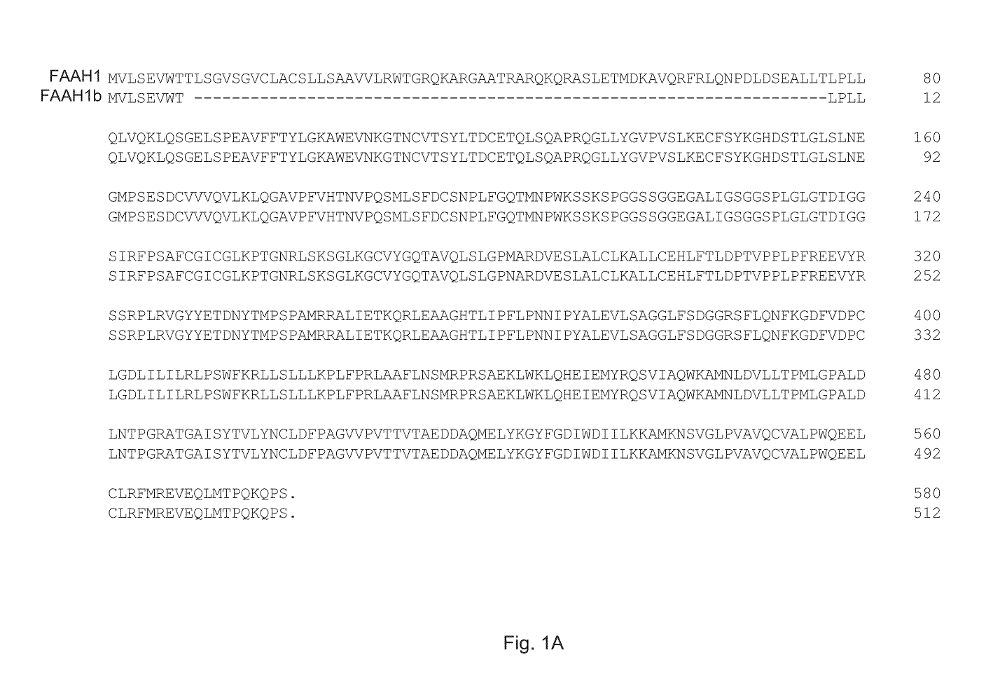

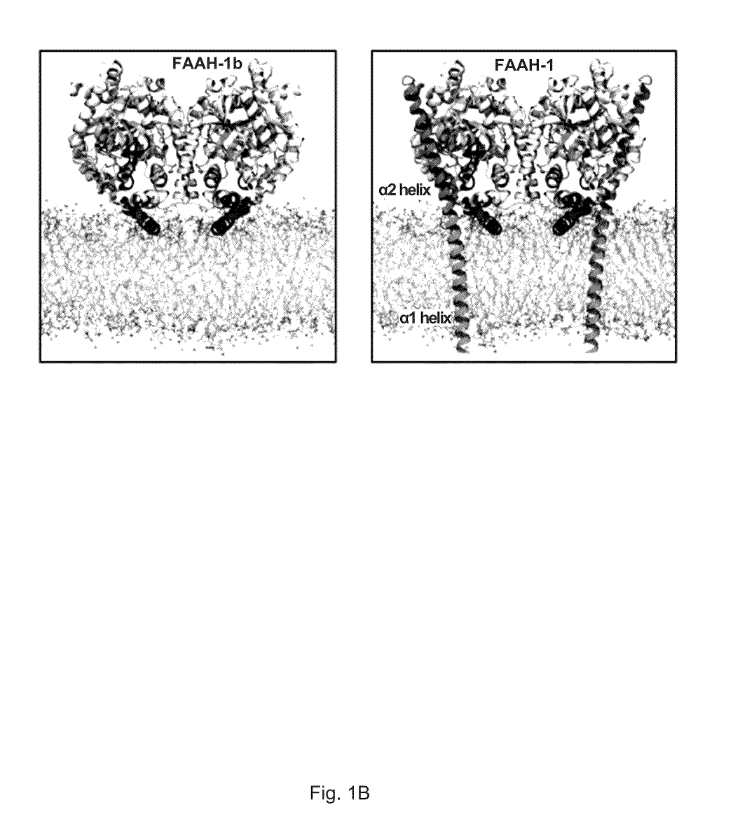

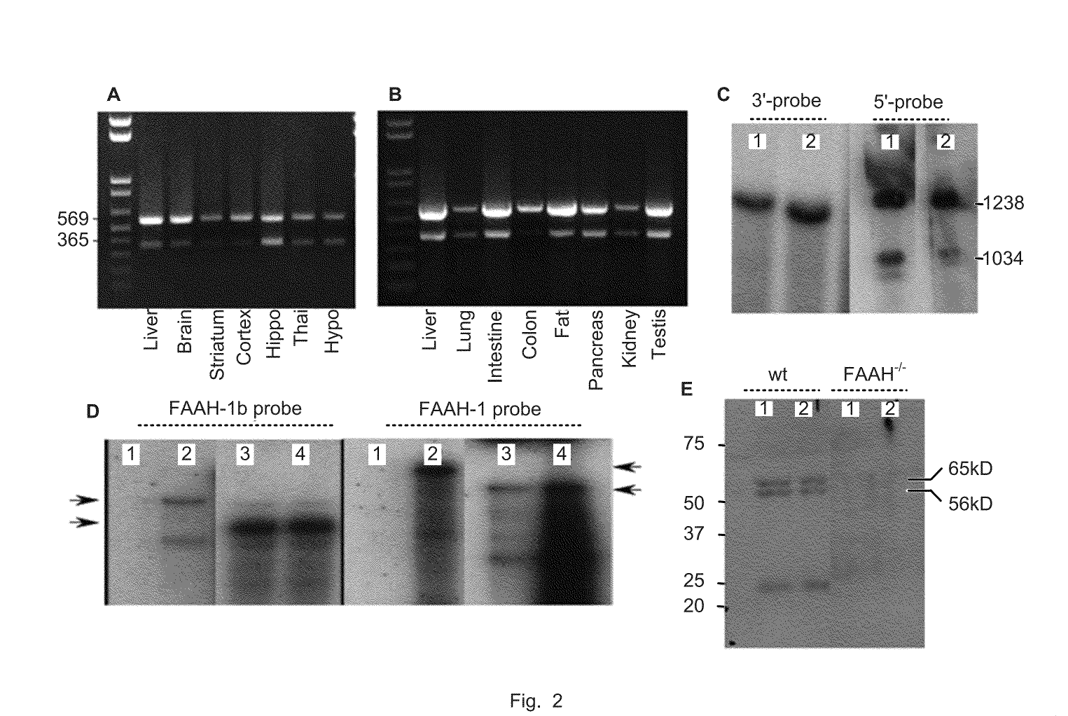

Total RNA was isolated from brain and other rat tissues, and amplified products of the faah-1 gene using reverse-transcriptase polymerase chain reaction. One of the complementary DNA products obtained was identical to faah-1 except that it lacked a 201-base pair segment encoding for amino-acid residues 9-76 (FIG. 1A,B). Southern blot analyses of cDNA generated by reverse transcription and ribonuclease protection assays confirmed the normal occurrence of FLAT mRNA in rat bra in (FIG. 2C,D). An antibody raised against the C-terminus of FAAH-1 identified in mouse brain extracts a band with an apparent molecular weight of 56 kDa, which is consistent with the calculated molecular weight of FLAT (56,008 Da) (FIG. 2E). No such band was found in brain extracts from FAAH-1-deficient mice (FIG. 2E), suggesting that FLAT might be a product of the faah-1 gene generated by alternative splicing at non-canonical sites (Montmayeur, J. P. et al., Science, 263, 95 (1994)). The predicted structure of FLAT lacks the first two a helices of FAAH-1: the α1 helix spans the lipid bilayer of intracellular membranes, while the α2 helix flanks the globular body of the protein exposed to the cytosol (FIG. 1B) (Bracey, M. H. et al., Science, 298, 1793 (2002)).

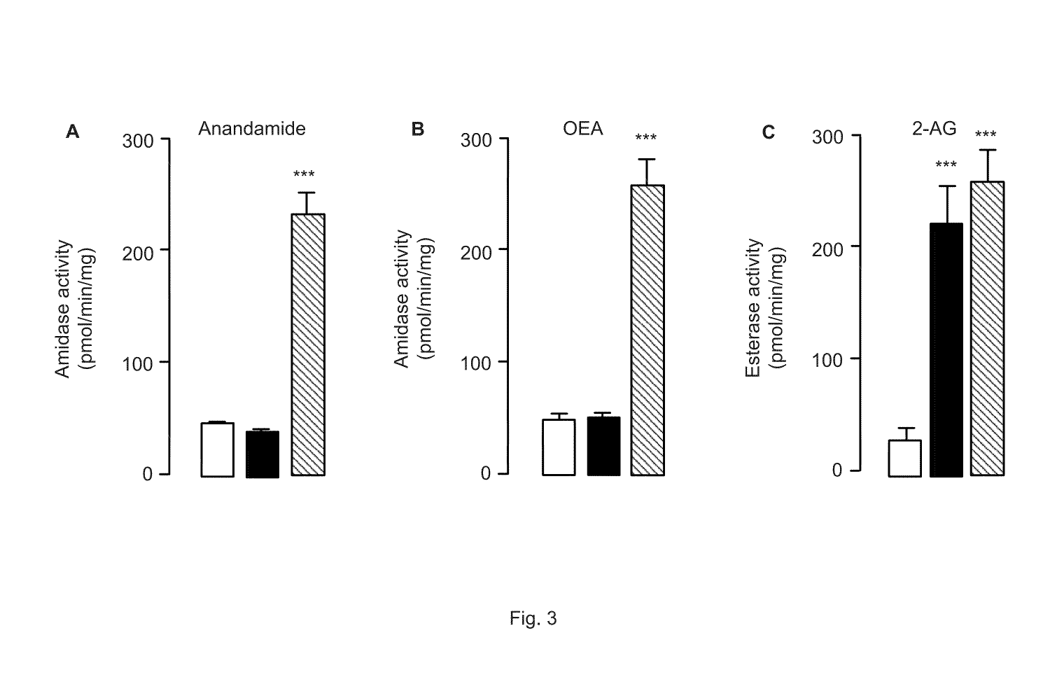

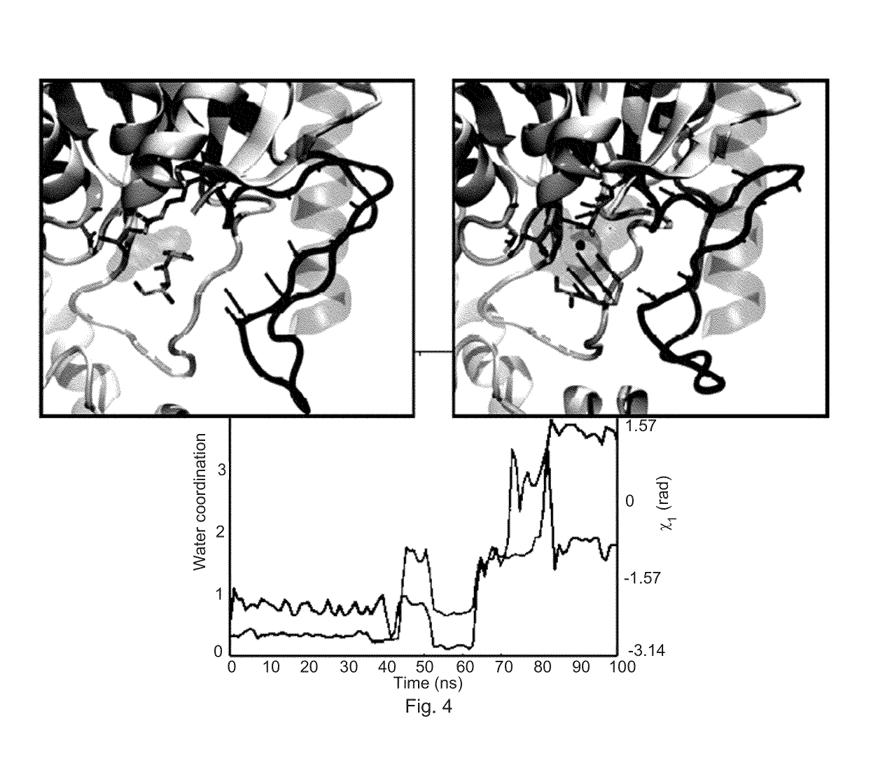

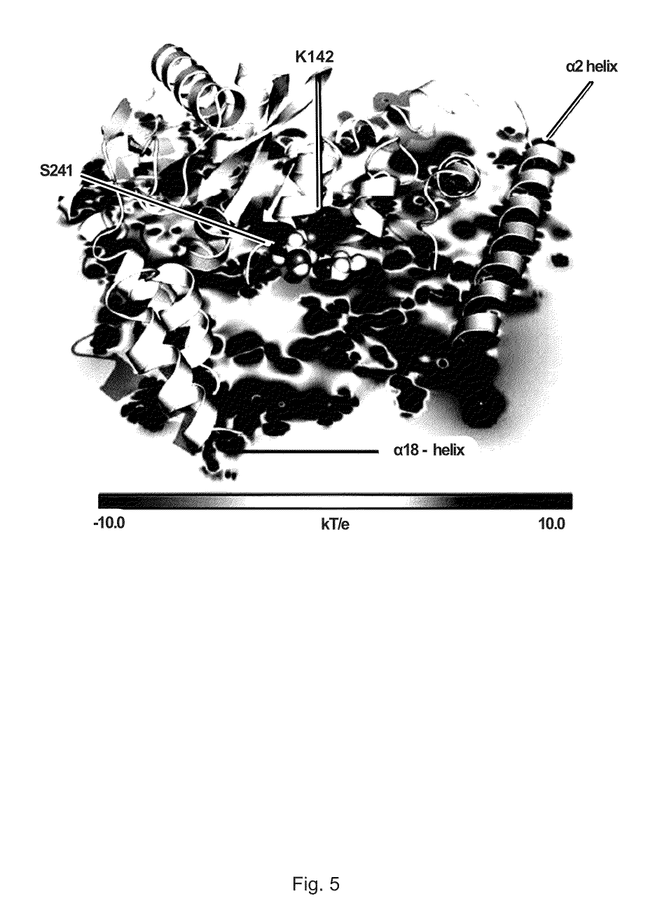

When expressed in Hek-293 cells, FLAT displayed no detectable amidase activity toward anandamide or OEA (FIG. 3A,B), suggesting that deletion of the α1 and α2 helices renders the protein catalytically defective. Computational studies identified two factors that might contribute to this effect: (i) increased flexibility of regions proximal to the missing helices—such as the ‘α2-interacting loop’ highlighted in FIG. 1B—could facilitate access of water to the catalytic site buried inside the enzyme's hydrophobic core (FIG. 4) (McKinney, M. K. et al., Annu Rev Biochem, 74, 411 (2005)); and (ii) deletion of the α2 helix, which carriers a positively charged surface, could lower the electrostatic potential in the region surrounding the catalytic triad component Lys⁷⁴ (corresponding to Lys¹⁴² in FAAH-1) (McKinney, M. K. et al., Annu Rev Biochem, 74, 411 (2005)) (FIG. 5). Both factors are expected to impair amidase activity by interrupting the proton transfer from Ser¹⁷³ (corresponding to catalytic Ser²⁴¹ in FAAH-1) to neutral Lys⁷⁴ (McKinney, M. K. et al., Annu Rev Biochem, 74, 411 (2005)). Though critical for the amidase activity of FAAH-1, Lys¹⁴² does not influence the ability of this enzyme to cleave ester substrates (Patricelli, M. P. et al., Biochemistry, 38, 14125 (1999)). Consistent with those data and the model proposed here, we found that recombinant FLAT effectively hydrolyzes the fatty acyl ester 2-oleoyl-sn-glycerol (FIG. 3B).

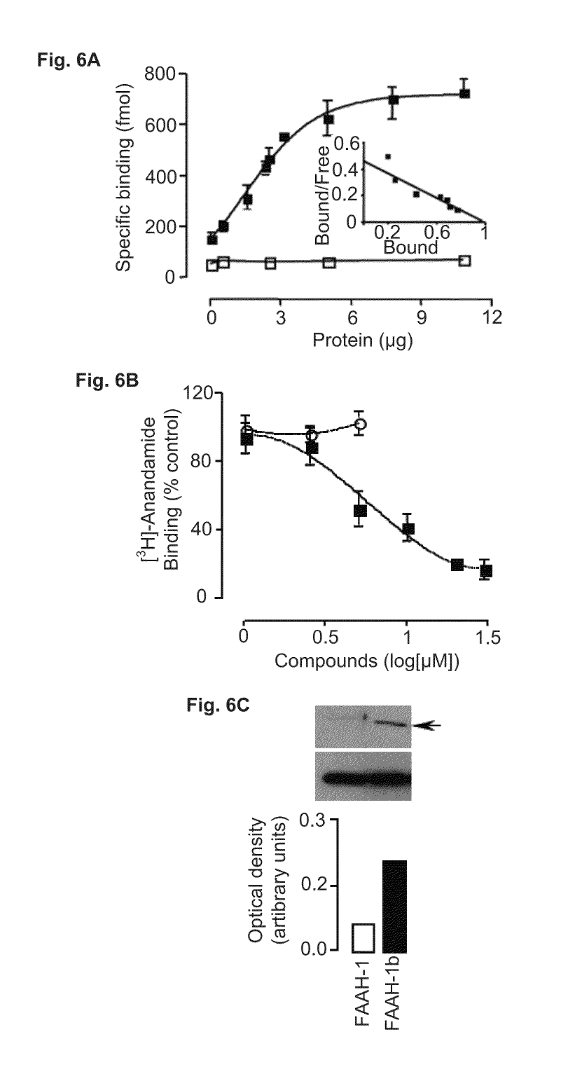

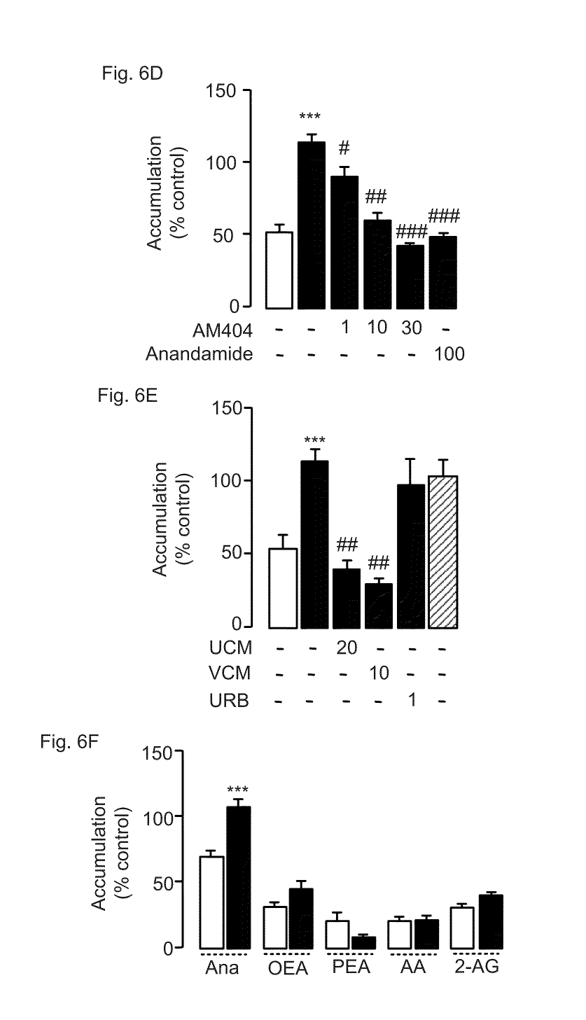

Our molecular dynamics simulations also suggested that FLAT might be able to bind anandamide, despite its inability to hydrolyze it (data not shown). To test this possibility, we expressed rat FLAT fused with glutathione-S-transferase (GST) in E. coli, and purified the recombinant protein by affinity chromatography. Saturation binding studies showed that [H³]-anandamide associates with FLAT-GST (dissociation constant, Kd=2 μM), but not with GST alone (FIG. 6A), and that this binding is displaced by the anandamide transport inhibitor AM404 (median inhibitory concentration, IC₅₀=5.3 μM) (FIG. 6B). By contrast, the FAAH inhibitor URB597 had no such effect (FIG. 6C), likely because its capacity to interact productively with FAAH-1 requires a fully functional catalytic triad (Alexander, J. P. et al., Chem Biol, 12, 1179 (2005)). Collectively, the experiments described above indicate that FLAT lacks amidase activity, but retains the ability to ligate anandamide.

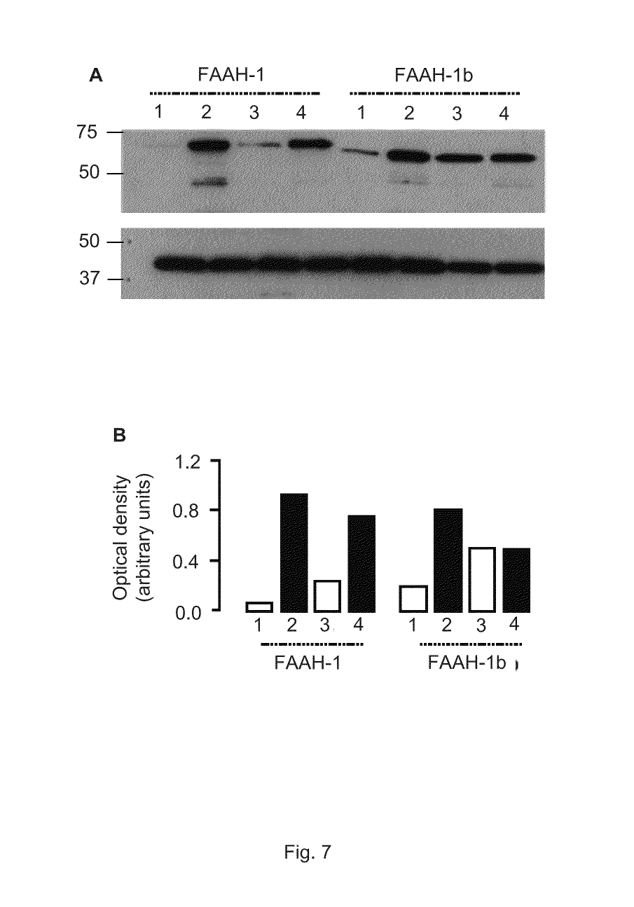

We detected significant amounts of FLAT in cytosolic fractions of transfected Hek293 cells (FIG. 6C), and found that the protein can be readily detached from membranes by incubation with sodium carbonate (0.1 M) (FIG. 7A-B). These findings, along with the observation that AM404 antagonizes the binding of [H³]-anandamide to FLAT, suggest a role for this protein in anandamide translocation. We examined therefore whether heterologous expression of FLAT might confer anandamide transport to Hek293 cells, which normally lack such an activity. We incubated control and FLAT-expressing cells for 5 min at 37° C. in a buffer containing [H³]-anandamide, and measured cell-associated radioactivity after removal of excess tracer (Beltramo, M. et al., Science, 277, 1094 (1997); Piomelli, D. et al., Proc Nall Acad Sci USA, 96, 5802 (1999)). Compared to controls, FLAT-expressing cells displayed a significantly higher level of [H³]-anandamide accumulation, which (i) was prevented by AM404 (IC₅₀=4 μM) and other transport inhibitors (VDM11, UCM707) as well as non-radioactive anandamide (100 μM) (FIG. 6D,E); and (ii) was selective for anandamide compared to four structurally related lipids: the FAAH substrates OEA and PEA, the eicosanoid precursor arachidonic acid, and the endocannabinoid ester 2-arachidonoyl-sn-glycerol (2-AG) (FIG. 6F). [H³]-Anandamide accumulation in FLAT-expressing Hek293 cells cannot be attributed to passive diffusion driven by FAAH-mediated hydrolysis, as it happens in some non-neural cell types, because the very low amidase activity present in native Hek293 cells was not increased by FLAT expression (FIG. 3A-C). Moreover, treatment with the FAAH inhibitor URB597 (1 μM) or mutation of Ser¹⁷³ (corresponding to the nucleophile Ser²⁴¹ in FAAH-1) did not affect [H³]-anandamide uptake by FLAT-expressing cells (FIG. 6E). These results suggest that FLAT facilitates anandamide translocation through a mechanism that is selective for anandamide, independent of amidase activity, and prevented by known inhibitors of anandamide transport.

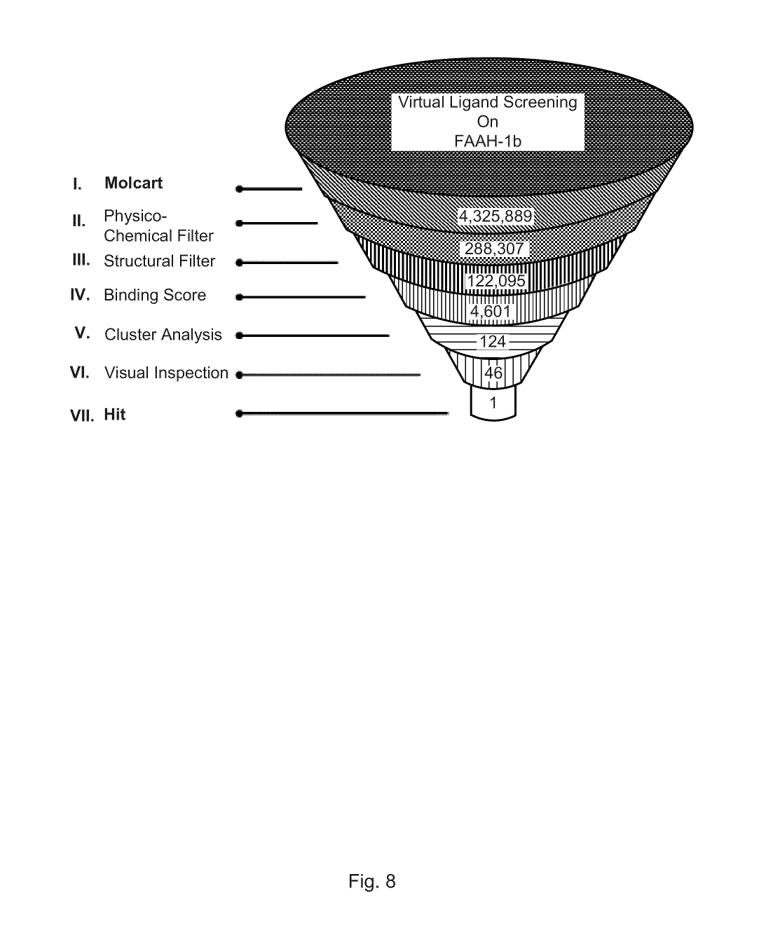

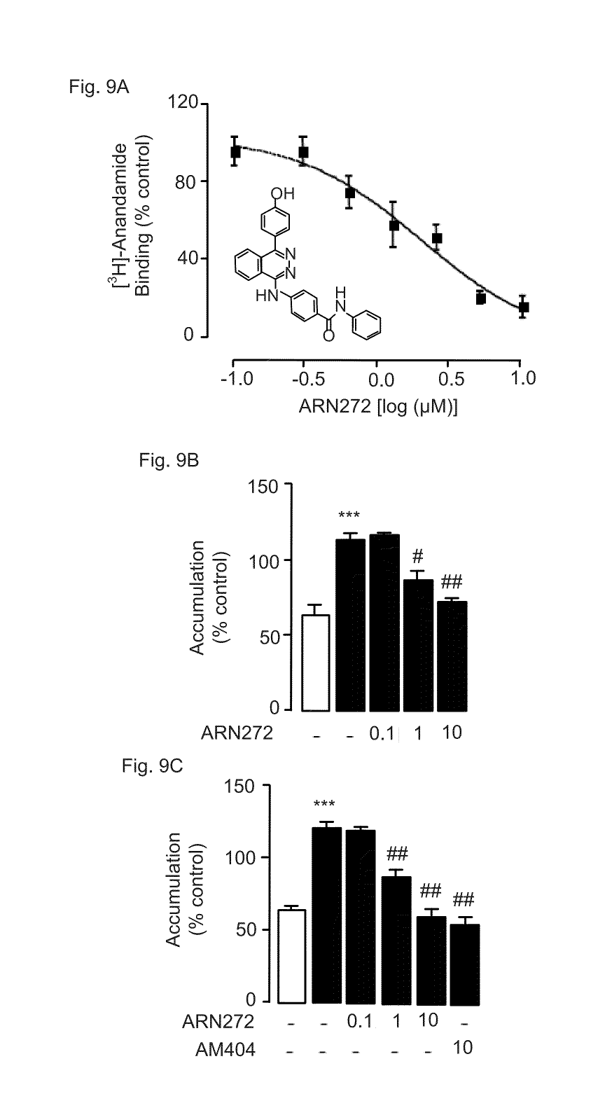

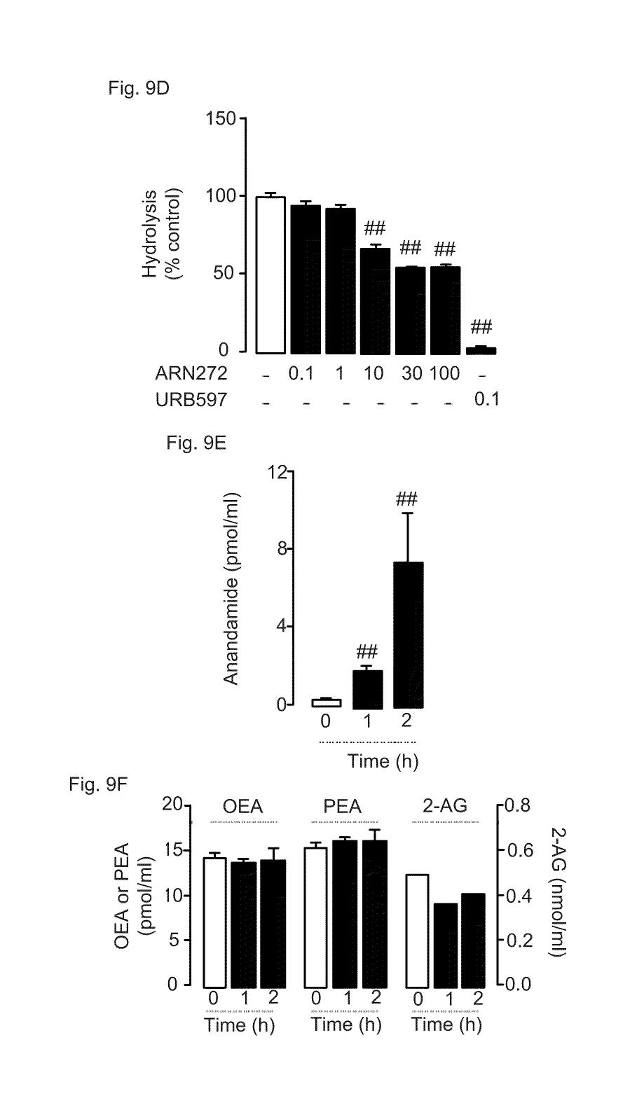

To further investigate the function of FLAT, we searched for small drug-like molecules that might interfere with its ability to sequester anandamide. Using an in silico approach, a virtual 4.3 million compound library was searched to a ligand-screening campaign structured in multiple steps of progressively increasing stringency (FIG. 8). The campaign returned a set of 46 structurally diverse compounds, which were tested for their interaction with FLAT in vitro. One of them, the substituted phthalazine ARN272, competitively antagonized [H³]-anandamide binding to purified recombinant FLAT (IC₅₀=1.8 μM; FIG. 9A) and inhibited [H³]-anandamide accumulation in both FLAT-expressing Hek293 cells (IC₅₀=3.5 μM; FIG. 9B) and rat cortical neurons in primary cultures (IC₅₀=1.5 μM;

FIG. 9C). Notably, ARN272 produced only a weak and incomplete inhibition of rat brain FAAH activity (50% at 100 μM; FIG. 9D) and was not significantly hydrolyzed by incubation with recombinant human FAAH-1 (≈5% hydrolyzed after 24 h at 25° C.). Consistent with these observations, and with results previously obtained with AM404 (Giuffrida, A. et al., Eur J Pharmacol, 408, 161 (2000)), we found that intraperitoneal administration of ARN272 (1 mg per kilogram of body weight) in mice increased plasma concentrations of anandamide (FIG. 9E) without changing the levels of OEA, PEA or 2-AG (FIG. 9F). The inhibitory effects of ARN272 on anandamide internalization in vitro and anandamide deactivation in vivo suggest that FLAT plays an obligatory role in the membrane translocation of this endocannabinoid transmitter.

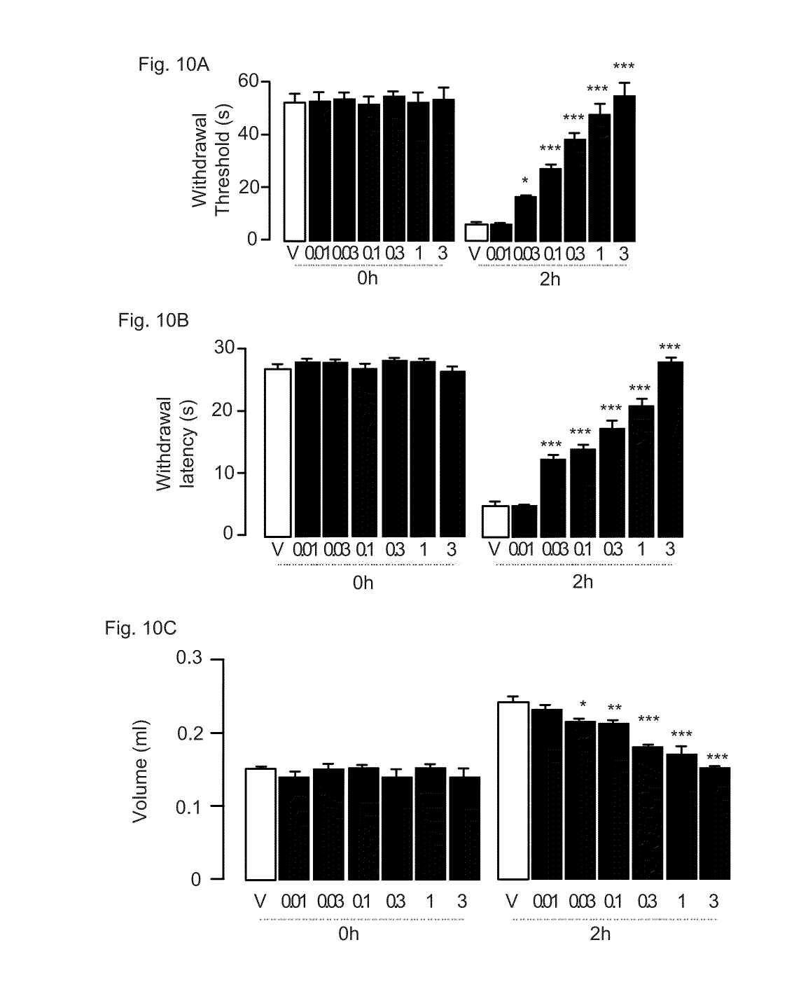

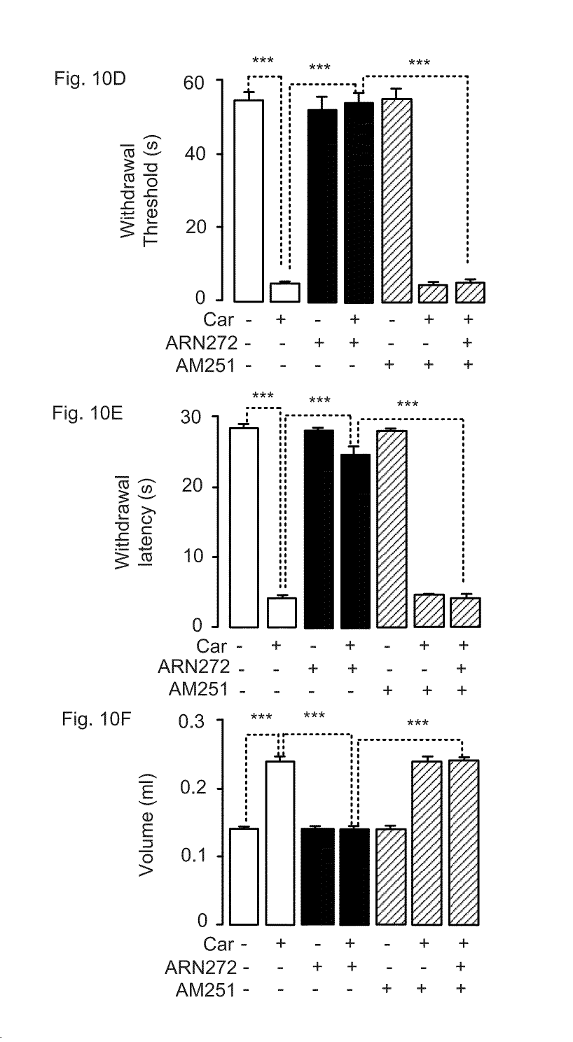

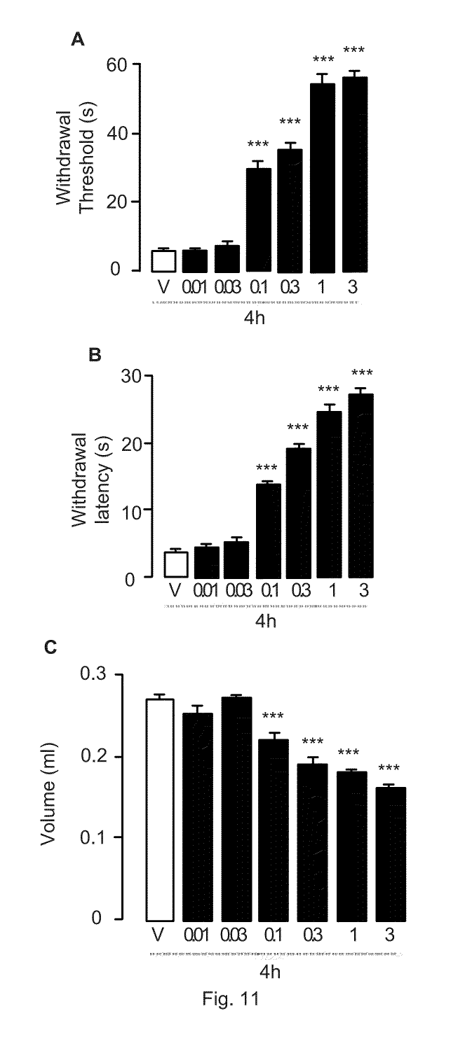

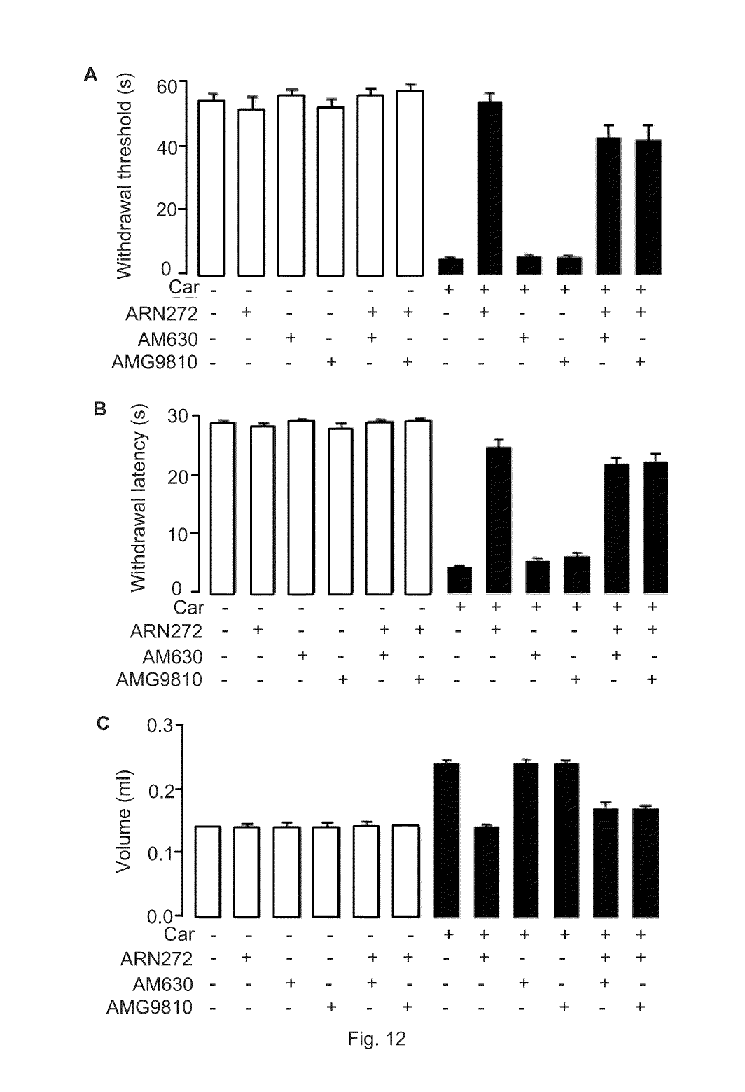

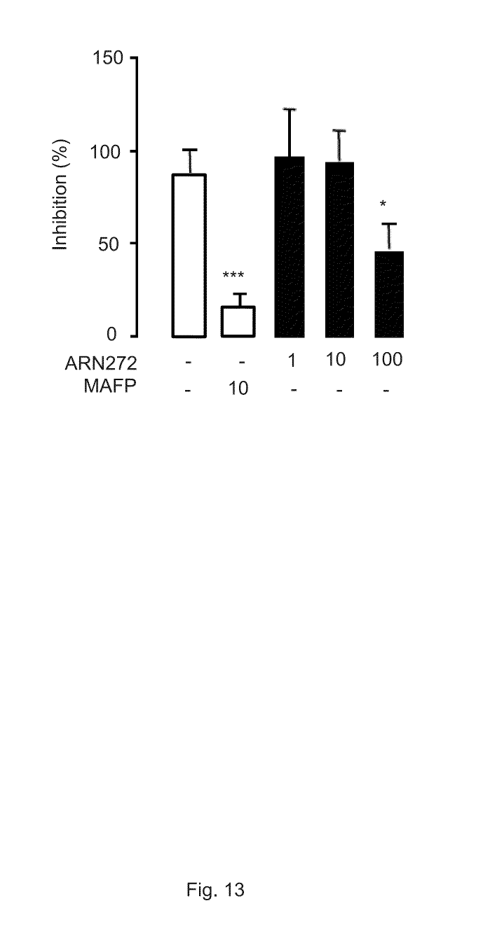

Anandamide transport inhibitors produce a variety of CB₁-mediated responses, which include analgesia in animal models of inflammatory pain (La Rana, G. et al., J Pharmacol Exp Ther, 317, 1365 (2006)). We tested therefore whether ARN272 might alleviate pain-related behaviors elicited in mice by intraplantar injection of the pro-inflammatory polysaccharide carrageenan. Intraperitoneal administration of ARN272 (0.01-3 mg per kilogram of body weight) caused a dose-dependent reduction of mechanical and thermal hyperalgesia in the inflamed paws of carrageenan-treated mice (FIG. 10A-B), which lasted up to 4 h (FIG. 11A-C). Moreover, treatment with ARN272 substantially reduced paw edema (FIG. 10C). The CB₁ receptor antagonist AM251 suppressed these effects (FIG. 10D-F), whereas the CB₂ antagonist AM630 and the transient receptor potential vanilloid-1 antagonist AMG9810 were ineffective (all drugs administered at 1 mg per kilogram of body weight, intraperitoneal) (FIG. 12A-C). ARN272 did not significantly alter the binding of the cannabinoid agonist [³H]-CP55940 to rat brain membranes (FIG. 13), suggesting that the anti-hyperalgesic actions of ARN272 resulted from inhibition of anandamide transport rather than direct stimulation of CB₁ receptors.

What is claimed is:

1. A pharmaceutical composition comprising:
a) a compound having the formula: 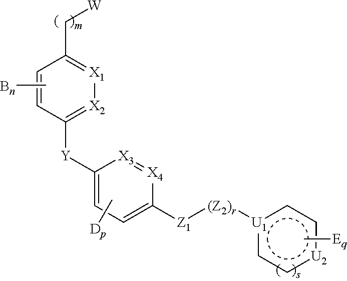
wherein:
W is aryl, heteroaryl, heterocycloalkyl, or alkyl, wherein the aryl, heteroaryl, or heterocycloalkyl can be substituted by 1 to 3 substituents selected, from lower alkyl, alkenyl, OH, alkoxy, cyano, halogen, NR¹R², NR¹COR², CONR¹R², wherein R¹ and R² are independently selected from H or lower alkyl;
m is an integer from 0 to 1;
X₁, X₂, X₃, and X₄ are independently selected from carbon and nitrogen;
n is an integer from 0 to 2,
each B member is independently selected from the group consisting of alkoxy, alkyl, alkenyl, halogen and heteroalkyl, and, optionally, when n is 2 and two B members are on adjacent carbon atoms, the two adjacent B members may be taken together along with the atoms to which they are attached to form a saturated, or unsaturated ring comprising 5 to 6 ring atoms;
Y is independently selected from O, NR³, and C═O, wherein R³ is hydrogen or lower alkyl;
p is an integer from 0 to 4;
each D member is independently selected from the group consisting of alkoxy, alkyl, alkenyl, halogen, and heteroalkyl, and optionally when two D members are on adjacent carbon atoms, the adjacent D members may be taken together along with the atoms to which they are attached to form a saturated or unsaturated ring comprising 5 to 6 ring atoms;
Z₁ and Z₂ are independently selected from C═O and NR⁴, wherein R⁴ is hydrogen or lower alkyl;
r is an integer from 0 to 1;
s is an integer from 0 to 1;
U₁ is independently selected from C or N;
U₂ is independently selected from C, O, and N(R⁵)ₜ, wherein the ring containing U₁ and U₂ can be aromatic, or partially or fully saturated, R⁵ is hydrogen or lower alkyl, and t is 0 or 1, with the proviso that when the ring containing U₁ and U₂ is aromatic, U₁ is C and t is 0;
q is an integer from 0 to 4;
each E member is independently selected from the group consisting of alkoxy, alkyl, alkenyl, halogen, and heteroalkyl, OH, cyano, NR⁶R⁷, wherein R⁶ and R⁷ are hydrogen or lower alkyl, and optionally when two E members are on adjacent carbon atoms the adjacent E members may be taken together along with the atoms to which they are attached to form a saturated or unsaturated ring comprising 5 to 6 ring atoms; and
b) a pharmaceutically acceptable excipient.

2. The composition of claim 1, wherein the compound has the formula:

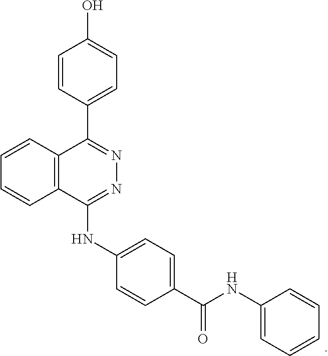 [SMILES: O=C(Nc1ccccc1)c1ccc(Nc2nnc(-c3ccc(O)cc3)c3ccccc23)cc1]

3. The composition of claim 1, wherein W is phenyl and m is 0.

4. The composition of claim 1, wherein Z₁ is C═O and Z₂ is NR³.

5. The composition of claim 1, wherein s is 1; r is 1; p is 0 or 1, n is 0, 1, or 2; and m is 0 or 1.

6. The composition of claim 1, wherein X₁ and X₂ are N and X₃ and X₄ are C.

7. The composition of claim 1, wherein U₁ and U₂ are C.

8. The composition of claim 1, wherein q is 0 or 1.

9. The composition of claim 1, wherein Z₂ is C═O and Z₁ is NR3.

10. The composition of claim 1, wherein Z₁ is C═O and Z₂ is NH and r is 1.

11. The composition of claim 1, comprising a compound of the formula: 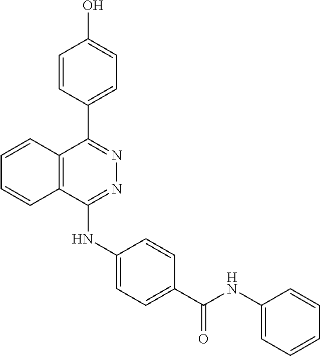 [SMILES: O=C(Nc1ccccc1)c1ccc(Nc2nnc(-c3ccc(O)cc3)c3ccccc23)cc1]
and a pharmaceutically acceptable excipient.

12. A method of modulating anandamide activity in a mammal, said method comprising administering to the mammal a compound having the formula: 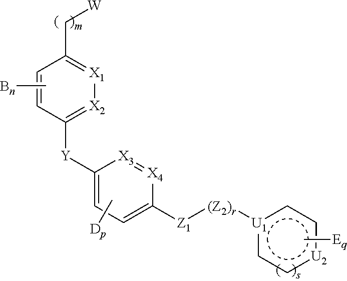
wherein:
W is aryl, heteroaryl, heterocycloalkyl, or alkyl, wherein the aryl, heteroaryl, or heterocycloalkyl can be substituted by 1 to 3 substituents selected from lower alkyl, alkenyl, OH, alkoxy, cyano, halogen, NR¹R², NR¹COR², CONR¹R², wherein R¹ and R² are independently selected from H or lower alkyl;
m is an integer from 0 to 1;
X₁, X₂, X₃, and X₄ are independently selected from carbon and nitrogen;
n is an integer from 0 to 2;
each B member is independently selected from the group consisting of alkoxy, alkyl alkenyl, halogen and heteroalkyl, and, optionally, when n is 2 and two B members are on adjacent carbon atoms, the two adjacent B members may be taken together along with the atoms to which they are attached to form a saturated or unsaturated ring comprising 5 to 6 ring atoms;
Y is independently selected from O, NR³, and C═O, wherein R³ is hydrogen or lower alkyl;
p is an integer from 0 to 4;
each D member is independently selected from the group consisting of alkoxy, alkyl, alkenyl, halogen, and heteroalkyl, and optionally when two D members are on adjacent carbon atoms, the adjacent D members may be taken together along with the atoms to which they are attached to form a saturated or unsaturated ring comprising 5 to 6 ring atoms;
Z₁ and Z₂ are independently selected from C═O and NR⁴, wherein R⁴ is hydrogen or lower alkyl;
r is an integer from 0 to 1;
s is an integer from 0 to 1;
U₁ is independently selected from C or N;
U₂ is independently selected from C, O, and N(R⁵)ₜ, wherein the ring containing U₁ and U₂ can be aromatic, or partially or fully saturated, R⁵ is hydrogen or lower alkyl, and t is 0 or 1, with the proviso that when the ring containing U₁ and U₂ is aromatic, U₁ is C and t is 0;
q is an integer from 0 to 4;
each E member is independently selected from the group consisting of alkoxy, alkyl, alkenyl, halogen, and heteroalkyl, OH, cyano, NR⁶R⁷, wherein R⁶ and ^{R7} are hydrogen or lower alkyl, and optionally when two E members are on adjacent carbon atoms the adjacent E members may be taken together along with the atoms to which they are attached to form a saturated or unsaturated ring comprising 5 to 6 ring atoms.

13. A method of treating pain or inflammation in a mammal in need thereof, said method comprising administering to the mammal a compound having the formula: 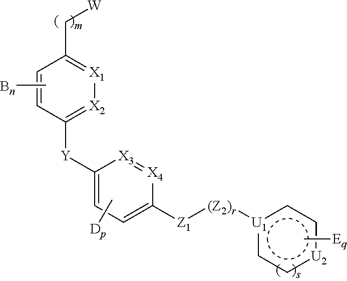
wherein:
W is aryl, heteroaryl, heterocycloalkyl, or alkyl, wherein the aryl, heteroaryl, or heterocycloalkyl can be substituted by 1 to 3 substituents selected from lower alkyl, alkenyl, OH, alkoxy, cyano, halogen, NR¹R², NR¹CO², CONR¹R², wherein R¹ and R² are independently selected from H or lower alkyl;
m is an integer from 0 to 1;
X₁, X₂, X₃, and X₄ are independently selected from carbon and nitrogen;
n is an integer from 0 to 2;
each B member is independently selected from the group consisting of alkoxy, alkyl, alkenyl, halogen and heteroalkyl, and, optionally, when n is 2 and two B members are on adjacent carbon atoms, the two adjacent B members may be taken together along with the atoms to which they are attached to form a saturated or unsaturated ring comprising 5 to 6 ring atoms;
Y is independently selected from O, NR³, and C═O, wherein R³ is hydrogen or lower alkyl;
p is an integer from 0 to 4;
each D member is independently selected from the group consisting of alkoxy, alkyl, alkenyl, halogen, and heteroalkyl, and optionally when two D members are on adjacent carbon atoms, the adjacent D members may be taken together along with the atoms to which they are attached to form a saturated or unsaturated ring comprising 5 to 6 ring atoms;
Z₁ and Z₂ are independently selected from C═O and NR⁴, wherein R⁴ is hydrogen or lower alkyl;
r is an integer from 0 to 1;
s is an integer from 0 to 1;
U₁ is independently selected from C or N;
U₂ is independently selected from C, O, and N(R⁵)ₜ, wherein the ring containing U₁ and U₂ can be aromatic, or partially or fully saturated, R⁵ is hydrogen or lower alkyl, and t is 0 or 1, with the proviso that when the ring containing U₁ and U₂ is aromatic, U₁ is C and t is 0;
q is an integer from 0 to 4;
each E member is independently selected from the group consisting of alkoxy, alkyl, alkenyl, halogen, and heteroalkyl, OH, cyano, NR⁶R⁷, wherein R⁶ and R⁷ are hydrogen or lower alkyl, and optionally when two E members are on adjacent carbon atoms the adjacent E members may be taken together along with the atoms to which they are attached to form a saturated or unsaturated ring comprising 5 to 6 ring atoms.

14. The method of claim 13, wherein the method is for treating acute or chronic inflammation in the mammal.

15. The method of claim 13, wherein the method is for treating acute or chronic pain in the mammal.

16. A method of modulating appetite or controlling body weight or treating nausea in a mammal, said method comprising administering to the mammal a compound having the formula: 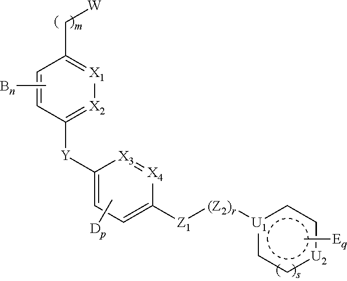
wherein:
W is aryl, heteroaryl, heterocycloalkyl, or alkyl, wherein the aryl, heteroaryl, or heterocycloalkyl can be substituted by 1 to 3 substituents selected from lower alkyl, alkenyl, OH, alkoxy, cyano, halogen, NR¹R², NR¹COR², CONR¹R², wherein R¹ and R² are independently selected from H or lower alkyl;
m is an integer from 0 to 1;
X₁, X₂, X₃, and X₄ are independently selected from carbon and nitrogen;
n is an integer from 0 to 2;
each B member is independently selected from the group consisting of alkoxy, alkyl, alkenyl, halogen and heteroalkyl, and, optionally, when n is 2 and two B members are on adjacent carbon atoms, the two adjacent B members may be taken together along with the atoms to which they are attached to form a saturated or unsaturated ring comprising 5 to 6 ring atoms;
Y is independently selected from O, NR³, and C═O, wherein R³ is hydrogen or lower alkyl;
p is an integer from 0 to 4;
each D member is independently selected from the group consisting of alkoxy, alkyl, alkenyl, halogen, and heteroalkyl, and optionally when two D members are on adjacent carbon atoms, the adjacent D members may be taken together along with the atoms to which they are attached to form a saturated or unsaturated ring comprising 5 to 6 ring atoms;
Z₁ and Z₂ are independently selected from C═O and NR⁴, wherein R⁴ is hydrogen or lower alkyl;
r is an integer from 0 to 1;
s is an integer from 0 to 1;
U₁ is independently selected from C or N;
U₂ is independently selected from C, O, and N(R⁵), wherein the ring containing U₁ and U₂ can be aromatic, or partially or fully saturated, R⁵ is hydrogen or lower alkyl, and t is 0 or 1, with the proviso that when the ring containing U₁ and U₂ is aromatic, U₁ is C and t is 0;
q is an integer from 0 to 4;
each E member is independently selected from the group consisting of alkoxy, alkyl, alkenyl, halogen, and heteroalkyl, OH, cyano, NR⁶R⁷, wherein R⁶ and R⁷ are hydrogen or lower alkyl, and optionally when two E members are on adjacent carbon atoms the adjacent E members may be taken together along with the atoms to which they are attached to form a saturated or unsaturated ring comprising 5 to 6 ring atoms.

17. A method of modulating a mood disorder, anxiety or depression in a mammal in need thereof, said method comprising administering to the mammal a compound having the formula: 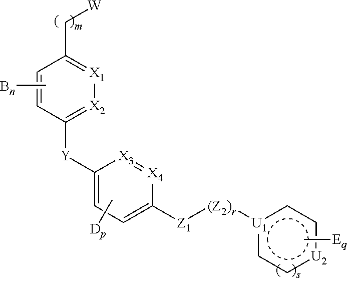
wherein:
W is aryl, heteroaryl, heterocycloalkyl, or alkyl, wherein the aryl, heteroaryl, or heterocycloalkyl can be substituted by 1 to 3 substituents selected from lower alkyl, alkenyl, OH, alkoxy, cyano, halogen, NR¹R², NR¹COR², CONR¹R² wherein R¹ and R² are independently selected from H or lower alkyl;
m is an integer from 0 to 1;
X₁, X₂, X₃, and X₄ are independently selected from carbon and nitrogen;
n is an integer from 0 to 2;
each B member is independently selected from the group consisting of alkoxy, alkyl, alkenyl, halogen and heteroalkyl, and, optionally, when n is 2 and two B members are on adjacent carbon atoms, the two adjacent B members may be taken together along with the atoms to which they are attached to form a saturated or unsaturated ring comprising 5 to 6 ring atoms;
Y is independently selected from O, NR³, and C═O, wherein R³ is hydrogen or lower alkyl;
p is an integer from 0 to 4;
each D member is independently selected from the group consisting of alkoxy, alkyl, alkenyl, halogen, and heteroalkyl, and optionally when two D members are on adjacent carbon atoms, the adjacent D members may be taken together along with the atoms to which they are attached to form a saturated or unsaturated ring comprising 5 to 6 ring atoms;
Z₁ and Z₂ are independently selected from C═O and NR⁴, wherein R⁴ is hydrogen or lower alkyl;
r is an integer from 0 to 1;
s is an integer from 0 to 1;
U₁ is independently selected from C or N;
U₂ is independently selected from C, O, and N(R⁵)ₜ, wherein the ring containing U₁ and U₂ can be aromatic, or partially or fully saturated., R³ is hydrogen or lower alkyl, and t is 0 or 1, with the proviso that when the ring containing U₁ and U₂ is aromatic, U₁ is C and t is 0;
q is an integer from 0 to 4;
each E member is independently selected from the group consisting of alkoxy, alkyl, alkenyl, halogen, and heteroalkyl, OH, cyano, NR⁶R⁷, wherein R⁶ and ^{R7} are hydrogen or lower alkyl, and optionally when two E members are on adjacent carbon atoms the adjacent E members may be taken together along with the atoms to which they are attached to form a saturated or unsaturated ring comprising 5 to 6 ring atoms.

18. A method of treating schizophrenia, post-traumatic stress, or a personality disorder in a patient, said method comprising administering to the patient a compound having the formula: 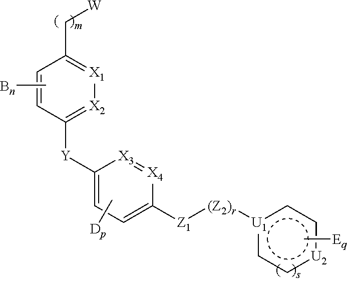
wherein:
W is aryl, heteroaryl, heterocycloalkyl, or alkyl, wherein the aryl, heteroaryl, or heterocycloalkyl can be substituted by 1 to 3 substituents selected from lower alkyl, alkenyl, OH, alkoxy, cyano, halogen, NR¹R², NR¹COR², CONR¹R², wherein R¹ and R² are independently selected from H or lower alkyl;
m is an integer from 0 to 1;
X₁, X₂, X₃, and X₄ are independently selected from carbon and nitrogen;
n is an integer from 0 to 2;
each B member is independently selected from the group consisting of alkoxy, alkyl, alkenyl, halogen and heteroalkyl, and, optionally, when n is 2 and two B members are on adjacent carbon atoms, the two adjacent B members may be taken together along with the atoms to which they are attached to form a saturated or unsaturated ring comprising 5 to 6 ring atoms;
Y is independently selected from O; NR³, and C═O, wherein R³ is hydrogen or lower alkyl;
p is an integer from 0 to 4;
each D member is independently selected from the group consisting of alkoxy, alkyl, alkenyl, halogen, and heteroalkyl, and optionally when two D members are on adjacent carbon atoms, the adjacent D members may be taken together along with the atoms to which they are attached to form a saturated or unsaturated ring comprising 5 to 6 ring atoms;
Z and Z₂ are independently selected from C═O and NR⁴, wherein R⁴ is hydrogen or lower alkyl;
r is an integer from 0 to 1;
s is an integer from 0 to 1;
U₁ is independently selected from C or N;
U₂ is independently selected from C, O, and N(R⁵)ₜ, wherein the ring containing U₁ and U₂ can be aromatic, or partially or fully saturated, R⁵ is hydrogen or lower alkyl, and t is 0 or 1, with the proviso that when the ring containing U₁ and U₂ is aromatic, U₁ is C and t is 0;
q is an integer from 0 to 4;
each E member is independently selected from the group consisting of alkoxy, alkyl, alkenyl, halogen, and heteroalkyl, OH, cyano, NR⁶R⁷, wherein R⁶ and R⁷ are hydrogen or lower alkyl, and optionally when two E members are on adjacent carbon atoms the adjacent E members may be taken to ether along with the atoms to which they are attached to form a saturated or unsaturated ring comprising 5 to 6 ring atoms.

19. A method of treating substance abuse and addiction in a mammal in need thereof, said method comprising administering to the mammal a compound having the formula: 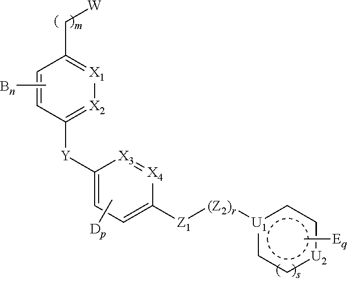
wherein:
W is aryl, heteroaryl, heterocycloalkyl, or alkyl, wherein the aryl, heteroaryl, or heterocycloalkyl can be substituted by 1 to 3 substituents selected from lower alkyl, alkenyl, OH, alkoxy, cyano, halogen, NR¹R², NR¹COR², CONR¹R², wherein R¹ and R² are independently selected from H or lower alkyl;
m is an integer from 0 to 1;
X₁, X₂, X₃, and X₄ are independently selected from carbon and nitrogen;
n is an integer from 0 to 2;
each B member is independently selected from the group consisting of alkoxy, alkyl, alkenyl, halogen and heteroalkyl, and, optionally, when n is 2 and two B members are on adjacent carbon atoms, the two adjacent B members may be taken together along with the atoms to which they are attached to form a saturated, or unsaturated ring comprising 5 to 6 ring atoms;
is independently selected from O, NR³ and C═O, wherein is hydrogen or lower alkyl;
p is an integer from 0 to 4;
each D member is independently selected from the group consisting of alkoxy, alkyl, alkenyl, halogen, and heteroalkyl, and optionally when two D members are on adjacent carbon atoms, the adjacent D members may be taken together along with the atoms to which they are attached to form a saturated or unsaturated ring comprising 5 to 6 ring atoms;
Z₁ and Z₂ are independently selected from C═O and NR⁴, wherein R⁴ is hydrogen or lower alkyl;
r is an integer from 0 to 1;
s is an integer from 0 to 1;
U₁ is independently selected from C or N;
U₂ is independently selected from C, O, and N(R⁵)ₜ, wherein the ring containing U₁ and U₂ can be aromatic, or partially or fully saturated, R⁵ is hydrogen or lower alkyl, and t is 0 or 1, with the proviso that when the ring containing U₁ and U₂ is aromatic, U₁ is C and t is 0;
q is an integer from 0 to 4;
each E member is independently selected from the group consisting of alkoxy, alkyl, alkenyl, halogen, and heteroalkyl, OH, cyano, NR⁶R⁷, wherein R⁶ and R⁷ are hydrogen or lower alkyl, and optionally when two E members are on adjacent carbon atoms the adjacent E members may be taken together along with the atoms to which they are attached to form a saturated or unsaturated ring comprising 5 to 6 ring atoms.

20. The method of claim 19, wherein the abuse or addiction is tobacco abuse or addiction.

21. A cDNA encoding FLAT.

22. An isolated mRNA encoding FLAT and not FAAH1.

23. A recombinant cell wherein the cell contains a heterologous nucleic acid encoding FLAT and the cell expresses the FLAT.

24. A method of screening a compound for activity as a modulator of FLAT, said method comprising contacting the compound with FLAT and measuring the ability of the compound to bind to FLAT or modulate the activity of the FLAT, wherein the FLAT is recombinant FLAT.

25. An isolated and/or recombinant FLAT protein of SEQ ID NO:2.

26. A method of testing a compound for the ability to modulate anandamide transport, said method comprising contacting the compound with an isolated or recombinant FLAT protein of SEQ ID NO:2 under conditions where the compound can bind the FLAT protein and determining whether the compound binds the FLAT protein.

27. The method of claim 26, wherein the compound is a compound of Formula I.
